(19) United States
(12) Patent Application Publication
(10) Pub. No.: US 2026/0345557 A1
(43) Pub. Date: Nov. 5, 2026

(54) ONDANSETRON TREATMENT OF ALCOHOL USE DISORDER

(71) Applicant: Adial Pharmaceuticals, Inc., Glen Allen, VA (US)

(72) Inventor: Schuyler VINZANT, Glen Allen, VA (US)

(21) Appl. No.: 19/653,528

(22) Filed: Apr. 21, 2026

Related U.S. Application Data
(63) Continuation of application No. PCT/US2025/036428, filed on Jul. 3, 2025.
(60) Provisional application No. 63/668,126, filed on Jul. 5, 2024.

(51) Int. Cl. A61K 31/4178; A61P 25/32

(52) U.S. Cl. CPC A61K 31/4178; A61P 25/32

(57) ABSTRACT

The disclosure relates to the field of alcohol use disorder. The disclosure provides methods of treating a subject having alcohol use disorder that includes administering ondansetron to a subject having a specified genotype corresponding to genetic variants that affect the 5-HT3 receptor complexes to which ondansetron binds and/or genetic variants that affect the expression of the serotonin transporter.

### CROSS-REFERENCE TO RELATED APPLICATIONS

This application is a continuation of PCT Application No. PCT/US2025/036428, filed Jul. 3, 2025, which claims the benefit of Provisional Patent Application No. 63/668,126, filed Jul. 5, 2024, each of which is incorporated herein by reference in its entirety.

### BACKGROUND

The disclosure relates to the field of alcohol use disorder. Specifically, the disclosure relates to methods of treating alcohol use disorder through the administration of ondansetron.

Alcohol use disorder (AUD) is among the leading causes of morbidity and mortality worldwide. Alcohol is an addictive substance that contributes to millions of deaths and disabilities worldwide (World Health Organization. Health Topics/Alcohol/Overview at www who.int/health-topics/alcohol). Excessive alcohol use accounts for 5.1% of the global disease burden and is the primary risk factor for premature mortality and disability in people aged 15 years to 49 years and accounts for 10% of all deaths in this age group (Id.). Over 95 million people live with alcohol dependence globally. Vulnerable societies have higher rates of alcohol-related fatalities and hospitalizations (Id.). Despite the serious global disease burden, only a minority of individuals with alcohol use disorder (AUD) receive pharmacotherapy due to concerns over their efficacy and the risk of clinically significant or severe adverse events (see, e.g., Mark et al., Drug Alcohol Depend 99(1-3):345-349 (2009); Rubinsky et al., J. Psychiatr. Res. 69:150-157 (2015); and Vayr et al., Drug Alcohol Depend. 199:116-121 (2019).

Accordingly, new modalities for treating AUD are needed. The methods provided herein address these needs.

### BRIEF SUMMARY

The disclosure provides methods of treating a subject having alcohol use disorder that includes administering ondansetron to a subject having a specified genotype corresponding to genetic variants that effect the 5-HT3 receptor complexes to which ondansetron binds (e.g., variants in the 5-HT3A, 5-HT3B and 5HT3AB receptors) and/or genetic variants that effect the expression of the serotonin transporter (e.g., variants in the, 5-HTTLPR and/or 3′UTR of the SLC6A4 gene that encodes the serotonin transporter). In some embodiments, the disclosure provides a method of altering the drinking habit and/or treating alcohol dependency of a subject who is a heavy drinker. In additional embodiments, the disclosure provides a method of predicting the likelihood that a subject having alcohol use disorder who is a heavy drinker will achieve an altered drinking habit, reduced alcohol dependency, and/or decreased drinking days upon the administration of ondansetron according to the provided methods.

In some embodiments, the disclosure provides:
[1] a method of treating a subject having alcohol use disorder to achieve one or more (preferably, two or more, three or more, or four or more) of the following effects (a)-(e) in one or both of the last two months of a treatment period, if the treatment period is five months or below, or in month 5 and/or month 6 if the treatment period is six months or longer:
   (a) zero heavy drinking days (HDDs), wherein an HDD is a day with ethanol consumption of 60 grams or more for males and 40 grams or more for females;
   (b) a reduction of percent heavy drinking days (PHDD) of at least 10% in addition to placebo effect, compared to that observed for the 4 weeks prior to the treatment period (“baseline”);
   (c) at least two no alcohol drinking days (“NDDs” or “abstinent days”) in addition to placebo effect;
   (d) a reduction of total alcohol consumption of at least 5% in addition to placebo effect, compared to baseline; and
   (e) a reduction of at least one drink per drinking day, in addition to placebo effect, compared to baseline, wherein each drink contains about 10 grams of ethanol;
the method comprising administering to the subject an effective amount of ondansetron, wherein: the subject is characterized as having the following baseline characteristics:
   (1) ≥6 HDDs;
   (2) consuming less than 10 drinks per drinking day;
   (3) having an average alcohol consumption at a medium risk level, which is ≥40 grams of ethanol per day for males and ≥20 grams of ethanol per day for females; and
   (4) ≤14 consecutive abstinent days; and
      the subject (i) has a genotype of: AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (ii) does not have a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
[2] the method of [1], wherein the subject does not have a genotype of LL/TT, which is rs4795541-LL genotype of the insertion-deletion polymorphism (5′-HTTLPR) in the 5′-regulatory region and rs1042173-TT SNP in the 3′-untranslated region of SLC6A4 gene that encodes the serotonin transporter;
[3] the method of [1] or [2], wherein the subject has a genotype of AG and at least one other SNP at the HTR3 AB complex;
[4] the method of [1] or [2], wherein the subject has a genotype of AG and AC (rs17614942-AC in HTR3B);
[5] the method of any of [1]-[4], wherein during the treatment period, the ondansetron is administered to the subject daily in an amount ranging from about 0.25 mg to about 1 mg;
[6] the method of any of [1]-[5], wherein during the treatment period, the ondansetron is administered to the subject twice a day, with each dose of about 0.33 mg;
[7] the method of any of [1]-[6], wherein the treatment period is at least six months, e.g., at least 9 months, at least 12 months, at least 24 months, such as 6-12 months, or 9-12 months, etc.;
[8] the method of [7], wherein the ondansetron is administered to the subject such that the subject has zero HDD in month 6 of the treatment period;
[9] the method of [7] or [8], wherein the ondansetron is administered to the subject such that the subject has a reduction of percent heavy drinking days in months 5 and/or 6 of the treatment period by at least 10%, preferably, at least 12% in addition to placebo effect, compared to baseline, such as a reduction of 15-35% in addition to placebo effect, compared to baseline;
[10] the method of any of [7]-[9], wherein the ondansetron is administered to the subject such that the subject has 2-4 days of no drinking days in addition to placebo effect in month 6 of the treatment period;
[11] the method of any of [7]-[10], wherein the ondansetron is administered to the subject such that the subject has a reduction of total alcohol consumption of 7-30% in addition to placebo effect in month 6 of the treatment period compared to baseline;
[12] the method of any of [7]-[11], wherein the ondansetron is administered to the subject such that the subject has a reduction of 1-3 drinks per drinking day in addition to placebo effect in month 6 of the treatment period compared to baseline;
[13] a method of treating alcohol use disorder, the method comprising
   administering ondansetron to a subject in need of treatment who belongs to a patient population characterized as (i) being heavy drinkers; and (ii) having a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (iii) not having a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
   wherein the ondansetron is administered to the subject with a dosing regimen that is effective in treating the patient population such that the percentage of the patient population having zero HDD (PNHDD) in one or both of the last two months of a treatment period, if the treatment period is five months or below, or in month 5 and/or month 6 if the treatment period is six months or longer, is higher than that of a placebo-controlled patient population, wherein an HDD is a day with ethanol consumption of 60 grams or more for males and 40 grams or more for females, for example, the percentage of the patient population having zero HDD is at least 10%, 20%, 30%, 40%, or 50% higher than that of the placebo-controlled patient population, e.g., 25-50%, 30-40%, or 30-35% higher than that of the placebo-controlled patient population;
   wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days;
[14] a method of increasing the likelihood of achieving zero heavy drinking days (HDDs) in one or both of the last two months of a treatment period, if the treatment period is five months or below, or in month 5 and/or month 6 if the treatment period is six months or longer, wherein an HDD is a day with ethanol consumption of 60 grams or more for males and 40 grams or more for females, the method comprising:
   administering ondansetron to a subject who belongs to a patient population characterized as (i) being heavy drinkers; and (ii) having a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (iii) not having a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
   wherein the ondansetron is administered to the subject with a dosing regimen that is effective in treating the patient population such that the percentage of the patient population having zero HDD (PNHDD) in one or both of the last two months of the treatment period, if the treatment period is five months or below, or in month 5 and/or month 6 if the treatment period is six months or longer, is higher than that of a placebo-controlled patient population, for example, the percentage of the patient population having zero HDD is at least 10%, 20%, 30%, 40%, or 50% higher than that of the placebo-controlled patient population e.g., 25-50%, 30-40%, or 30-35% higher than that of the placebo-controlled patient population;
   wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days;
[15] the method of [13] or [14], wherein all subjects of the patient population have a genotype of AG and at least one other SNP at the HTR3 AB complex;
[16] the method of [13] or [14], wherein all subjects of the patient population have a genotype of AG and AC (rs17614942-AC in HTR3B);
[17] the method of any of [13]-[16], wherein no subject of the patient population has a genotype of LL/TT, which is rs4795541-LL genotype of the insertion-deletion polymorphism (5′-HTTLPR) in the 5′-regulatory region and rs1042173-TT SNP in the 3′-untranslated region of SLC6A4 gene that encodes the serotonin transporter;
[18] the method of any of [13]-[17], wherein the treatment period is at least six months, e.g., at least 9 months, at least 12 months, at least 24 months, such as 6-12 months, or 9-12 months, etc.;
[19] the method of [18], wherein the dosing regimen is effective in treating the patient population to achieve a reduction of percent heavy drinking days (PHDD) in months 5 and/or 6 of the treatment period compared to baseline, wherein the reduction of PHDD in the patient population is statistically significantly higher than that of the placebo-controlled patient population or a patient population having only a single SNP selected from AC alone, AG alone, or GG alone;
[20] the method of [18] or [19], wherein the dosing regimen is effective in treating the patient population to achieve 2-4 more days of no drinking days (NDD) in months 5 and/or 6 of the treatment period higher than that of the placebo-controlled patient population;
[21] the method of any of [18]-[20], wherein the dosing regimen is effective in treating the patient population to achieve a reduction of total alcohol consumption (TAC) in months 5 and/or 6 of the treatment period, wherein the reduction of TAC in the patient population is higher than that of the placebo-controlled patient population;
[22] the method of any of [18]-[21], wherein the dosing regimen is effective in treating the patient population to achieve a reduction of drinks per drinking day (DDD) in months 5 and/or 6 of the treatment period, wherein the reduction of DDD in the patient population is higher than that of the placebo-controlled patient population;
[23] the method of any of [18]-[22], wherein when compared to the placebo-controlled patient population, the dosing regimen is effective in treating the patient population to achieve (a) a reduction of percent heavy drinking days in months 5 and/or 6 of the treatment period compared to baseline by at least 12% greater, such as 15-35% greater; (b) 2-4 more days of no drinking days in month 6 of the treatment period; (c) a reduction of total alcohol consumption of in month 6 of the treatment period compared to baseline by 7-30% greater; and (d) a reduction of 1-3 more drinks per drinking day in month 6 of the treatment period compared to baseline;
[24] the method of any of [13]-[23], wherein during the treatment period, the ondansetron is administered to the subject daily in an amount ranging from about 0.25 mg to about 1 mg;
[25] the method of any of [13]-[24], wherein during the treatment period, the ondansetron is administered to the subject twice a day, with each dose of about 0.33 mg;
[26] the method of any of [13]-[25], further comprising determining or having determined the subject as belonging to the patient population;
[27] a method of altering drinking habit of a subject who is a heavy drinker and (1) has a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter, and/or (2) does not have a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter; the method comprising:
   administering ondansetron to the subject in an amount effective to achieve one or more (preferably, two or more, three or more, or four or more) of the following effects (a)-(e) in one or both of the last two months of a treatment period if the treatment period is five months or below, or in month 5 and/or month 6 if the treatment period is six months or longer, thereby altering the subject's drinking habit:
   (a) no heavy drinking days (HHDs), wherein an HDD is a day with ethanol consumption of 60 grams or more for males and 40 grams or more for females;
   (b) a reduction of percent heavy drinking days of at least 10% in addition to placebo effect compared to that observed for the 4 weeks prior to the treatment period (“baseline”);
   (c) at least two or more no drinking days in addition to placebo effect;
   (d) a reduction of total alcohol consumption of at least 5% in addition to placebo effect, compared to baseline; and
   (e) a reduction of drinks per drinking day of at least one drink in addition to placebo effect, compared to baseline, wherein each drink contains about 10 grams of ethanol,
      wherein the heavy drinker has the following baseline characteristics: ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days;
[28] a method of treating alcohol dependency of a subject who is a heavy drinker and (1) has a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter, and/or (2) does not have a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter; the method comprising:
   administering to the subject ondansetron in an amount effective to achieve one or more (preferably, two or more, three or more, or four or more) of the following (a)-(e), in one or both of the last two months of a treatment period if the treatment period is five months or below, or in month 5 and/or month 6 if the treatment period is six months or longer:
   (a) no heavy drinking days (HHDs), wherein an HDD is a day with ethanol consumption of 60 grams or more for males and 40 grams or more for females;
   (b) a reduction of percent heavy drinking days of at least 10% in addition to placebo effect compared to that observed for the 4 weeks prior to the treatment period (“baseline”);
   (c) at least two or more no drinking days in addition to placebo effect;
   (d) a reduction of total alcohol consumption of at least 5% in addition to placebo effect, compared to baseline; and
   (e) a reduction of drinks per drinking day of at least one drink in addition to placebo effect, compared to baseline, wherein each drink contains about 10 grams of ethanol,
   wherein the heavy drinker has the following baseline characteristics: ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days;
[29] a method of predicting the likelihood of a subject having alcohol use disorder achieving zero heavy drinking days (HDDs) in one or both of the last two months of a treatment period, if the treatment period is five months or below, or in month 5 and/or month 6 if the treatment period is six months or longer, wherein an HDD is a day with ethanol consumption of 60 grams or more for males and 40 grams or more for females, the method comprising:
   (1) administering ondansetron to a patient population characterized as (i) being heavy drinkers; and (ii) having a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (iii) not having a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
   (2) identifying the patient population as responsive to ondansetron treatment if the ondansetron can be administered with a dosing regimen that is effective in treating the patient population such that the percentage of the patient population having zero HDD (PNHDD) in one or both of the last two months of the treatment period, if the treatment period is five months or below, or in month 5 and/or month 6 if the treatment period is six months or longer, is higher than that of a placebo-controlled patient population, for example, at least 10%, 20%, 30%, 40%, or 50% higher than that of the placebo-controlled patient population, e.g., 25-50%, 30-40%, or 30-35% higher than that of the placebo-controlled patient population;
      wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days; and
   (3) determining or having determined whether the subject belongs to the patient population by comparing the genotype and drinking pattern with those of the patient population, wherein a subject belonging to the patient population has a higher chance of achieving zero HDD;
[30] a method of treating alcohol use disorder, the method comprising:
   (1) determining or having determined a subject in need of treatment as having a higher chance of achieving zero HDD according to the method of [29, and
   (2) administering the subject ondansetron with a dosing regimen that is effective in treating the patient population such that the percentage of the patient population having zero HDD (PNHDD) in one or both of the last two months of the treatment period, if the treatment period is five months or below, or in month 5 and/or month 6 if the treatment period is six months or longer, is higher than that of a placebo-controlled patient population, for example, at least 10%, 20%, 30%, 40%, or 50% higher than that of the placebo-controlled patient population, e.g., 25-50%, 30-40%, or 30-35% higher than that of the placebo-controlled patient population.

### BRIEF DESCRIPTION OF THE DRAWINGS/FIGURES

FIG. 1 shows trajectories of change for heavy drinking individuals having the AG genotype for heavy drinking days (HDD) during the treatment with ondansetron (AD04) or placebo (PBO).

FIG. 2 shows trajectories of change for heavy drinking individuals having the AC genotype for heavy drinking days (HDD) during the treatment with ondansetron (AD04) or placebo (PBO).

FIG. 3 shows trajectories of change for heavy drinking individuals having the GG genotype for heavy drinking days (HDD) during the treatment with ondansetron (AD04) or placebo (PBO).

FIG. 4 shows trajectories of change for heavy drinking individuals having the LL/TT genotype for heavy drinking days (HDD) during the treatment with ondansetron (AD04) or placebo (PBO).

FIG. 5 shows trajectories of change for heavy drinking individuals having the AG+ genotype for zero heavy drinking days (HDD) during the treatment with ondansetron (AD04) or placebo (PBO).

FIG. 6 shows subgroup treatment effects in populations with baseline mean drinks per drinking day of less than 8 (based on 14 grams of ethanol per drink), treatment effects based on end point of no HDD at month 5 and 6.

### DETAILED DESCRIPTION

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Other features and advantages of the disclosed methods and compositions will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Recitation of ranges of values herein are merely intended to serve as a shorthand method of referring individually to each separate value falling within the range, unless otherwise indicated herein, and each separate value is incorporated into the specification as if it were individually recited herein.

Where embodiments of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed methods and compositions also envisage the explicit exclusion of one or more of any of the group members in the disclosed compositions or methods.

As used herein, “heavy drinker” refers to an individual who has the following: (greater than or equal to, i.e., at least) ≥6 heavy drinking days (HDDs) in the period of 4 weeks prior to treatment; a consumption of (less than) <10 drinks per drinking day (DDD) in the period of 4 weeks prior to treatment. In some embodiments, the “heavy drinker” can be further characterized as having an average alcohol consumption at a medium risk level, which is (greater than or equal to, i.e., at least) ≥40 grams of ethanol per day for a male and ≥20 grams of ethanol per day for a female in the period of 4 weeks prior to treatment; and/or having (less than or equal to, i.e., at most)≤14 consecutive abstinent (no drinking alcohol (ethanol)) days in the period of 4 weeks prior to treatment. To be clear, the average alcohol consumption at a medium risk level should be understood as less than 60 grams of ethanol per day for a male and 40 grams of ethanol per day for a female, which is the lower limit for a high risk level. A “heavy drinker” may or may not be dependent on alcohol and may or may not meet DSM V criteria for alcohol abuse. In some embodiments, a heavy drinker treated with the provided methods herein is characterized as having no symptoms of organ injuries (such as liver injuries) associated with drinking at baseline. In some embodiments, the methods provided herein reduce the likelihood of developing organ injuries in the heavy drinker.

As used herein, “patient population” refers to a set of individuals who have been screened for certain characteristics, who are treated in a standard manner during a treatment period, and who are monitored during the treatment period. When not otherwise indicated as placebo-controlled, the “patient population” refers to a set of individuals who are administered a substance being tested, e.g., ondansetron (AD04).

As used herein, the term “placebo-controlled patient population” refers to the patient population defined herein, of which the individuals are administered a placebo instead of a test substance e.g., ondansetron (AD04). A person of ordinary skill in the art would know how to select a “placebo-controlled patient population” for a given “patient population”. The concept of placebo-controlled is well understood in the art. Also, as well understood in the art, a placebo refers to a dosage form that is generally considered to be physiologically inert but otherwise considered to be the same as the dosage form containing the test substance, for example, the excipients, shape, size, etc. for the placebo and dosage form containing the test substance are generally the same.

As used herein, the term “placebo effect” refers to the mean effect observed in comparison with baseline for a patient population having the same baseline characteristics as a subject defined herein but is treated with a placebo, which is determined by a randomized, controlled clinical trial as described in this application. In embodiments herein, unless otherwise indicated or contrary from context, the term “placebo effect” refers to a reduction of 10-70% (e.g., 10%, 20%, 30%, 40%, 50%, 60%, 70%, or any ranges or values between the recited values, such as 10-40%, 20-60%, 35%, etc.) of an efficacy measure herein, such as PHDD, DDD, TAC, etc., compared to baseline. For example, in some embodiments, the placebo effect in reducing PHDD ranges from 10-40% in one or both of the last two months of a treatment period, or at month 5 and/or month 6 of a treatment period. As discussed in the Examples section, for the overall patient population, the placebo controlled group achieved a reduction of PHDD of about 38%. In some embodiments, the placebo effect in reducing total alcohol consumption (TAC) ranges from 30-70% in one or both of the last two months of a treatment period, or at month 5 and/or month 6 of a treatment period. In some embodiments, the placebo effect in reducing drinks per drinking day (DDD) ranges from 10-40% or 1-6 drinks per drinking day, in one or both of the last two months of a treatment period, or at month 5 and/or month 6 of a treatment period. In some embodiments, the placebo effect achieves 1-10 abstinent days, in one or both of the last two months of a treatment period, or at month 5 and/or month 6 of a treatment period.

As used herein, the term “treating” may include prophylaxis of a specific disease, disorder, or condition, or alleviation of the symptoms associated with a specific disease, disorder, or condition, and/or prevention or elimination of symptoms associated with a specific disease, disorder, or condition. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

As used herein, a total alcohol consumption (TAC) is the amount of alcohol (ethanol) consumed during a time period. For example, the total alcohol consumption (TAC) in a month can be the equivalent amount in grams of ethanol consumed by an individual over a month. For a patient population, the TAC refers to the average TAC of the patient population.

As used herein, “no drinking days” (NDD) (also referred to as “no alcohol drinking days” or “abstinent days”) refers to the number of days during each of the days an individual consumed no alcohol (ethanol), i.e., the numbers of days during which the individual was abstinent, during a time period. For example, if an individual is abstinent for 10 days out of a month, the number of “no drinking days” (NDD) would be 10 for that month. For a patient population or placebo-controlled patient population, the NDD refers to the average number of “no drinking days” of the patient population or placebo-controlled patient population.

As used herein, a “baseline” value or characteristic refers to a quantity or average of a quantity, or characteristic (such as drinking patterns), during a period prior to the beginning of treatment. Unless otherwise specified or contrary from context, the period prior to the beginning of treatment refers to the 4 weeks before the beginning of treatment. For a patient population or placebo-controlled patient population, a “baseline” value refers to the average value observed for the patient population or placebo-controlled patient population.

As used herein, “treatment period” refers to the period during which an individual, who may be part of a patient population or a placebo-controlled patient population, receives a substance being tested (e.g., ondansetron (AD04)) or a placebo. In some embodiments, the treatment period is six (6) months, at least 6 months, e.g., at least 9 months, at least 12 months, or at least 24 months. For example, the treatment period can be in the range of from six to twelve (6-12) months or in the range of nine to twelve (9-12) months.

As used herein, “percent heavy drinking days” (PHDD) is the number of heavy drinking days (HDD) divided by the total number of days in a period of time multiplied by 100. For example, the percent heavy drinking days (PHDD) observed for the 4 weeks prior to a treatment period (baseline) can be calculated by dividing the number of HDD by 4 weeks and multiplied by 100. For a patient population or placebo-controlled patient population, the PHDD refers to the average PHDD of the patient population or placebo-controlled patient population.

As used herein, “one drink per drinking day” (DDD) refers to consumption of one standard drink over one day.

As used herein, unless otherwise specified or contrary from context, a drink or standard drink is considered to contain about 10-14 grams of ethanol. A person of ordinary skill in the art would appreciate that in the clinical settings in the U.S., a drink or standard drink may be considered to contain about 14 grams of ethanol, which is used in some of the studies herein. A drink may be about 0.5 fluid ounces (oz) of pure (absolute) ethanol (alcohol), about 10 oz of beer, about 4 oz of wine, or about 1 oz of 100-proof liquor.

As used herein, a subject having an “AG” genotype refers to a subject having the single nucleotide polymorphism (SNP) rs1150226-AG in the gene (HTR3A) that encodes the 5-HT3A receptor subunit. A subject having a genotype of “AG alone” or “AG only” (“AG” in Table 2), refers to a subject that has the AG genotype with none of the GG, AC, LL, and TT genotypes observed. A subject having a genotype of “AG+” is AG positive and may or may not have an additional genotype. A subject having an AG genotype and at least one other SNP at the HTR3 AB complex is AG positive and is positive for another genotype, such as AC and/or GG.

As used herein, a subject having a “GG” genotype refers to a subject having the single nucleotide polymorphism (SNP) rs1176713-GG in the gene (HTR3A) that encodes the 5-HT3A receptor subunit. A subject having a genotype of “GG alone” or “GG only” (“GG” in Table 2), refers to a subject that has the GG genotype with none of the AG, AC, LL, and TT genotypes observed. A subject having a genotype of “GG+” is GG positive and may or may not have any additional genotyped. A subject having a GG genotype and at least one other SNP at the HTR3 AB complex is GG positive and is positive for another genotype, such as AC and/or AG.

As used herein, a subject having an “AC” genotype refers to a subject having the single nucleotide polymorphism (SNP) rs17614942-AC in the gene (HTR3B) that encodes the 5-HT3B receptor subunit. A subject having a genotype of “AC alone” or “AC only” (“AC” in Table 2), refers to a subject that has the AC genotype with none of the AG, GG, LL, and TT genotypes observed. A subject having a genotype of “AC+” is AC positive and may or may not have any additional genotypes. A subject having an AC genotype and at least one other SNP at the HTR3 AB complex is AC positive and is positive for another genotype, such as AG and/or GG.

As used herein, a subject having a genotype of AC and AG refers to a subject having the AC and AG genotypes. A subject having a genotype of “AC and AG alone” or “AC and AG only” (“AC/AG” in Table 2), refers to a subject that has the AC and AG genotypes with none of the GG, LL, and TT genotypes observed. A subject having a genotype of “AC/AG+” is AC and AG positive and may or may not have any additional genotypes. A subject having an AC and AG genotype and at least one other SNP at the HTR3 AB complex is AC and AG positive and is positive for another genotype, such as GG.

As used herein, a subject having an “LL” genotype refers to a subject having the rs4795541-LL insertion-deletion polymorphism (5′-HTTLPR) in the 5′ regulatory region of SLC6A4.

As used herein, a subject having a “TT” genotype refers to a subject having the rs1042173-TT single nucleotide polymorphism (SNP) in the 3′ untranslated region of the SLC6A4 gene that encodes the serotonin transporter.

As used herein, a subject having a genotype of LL and TT (“LL/TT”) refers to a subject having both the LL and TT genotypes. A subject having a genotype of “LL and TT alone” or “LL and TT only” (“LL/TT” in Table 2), refers to a subject that has the LL and TT genotypes with none of the AG, GG, and AC genotypes observed. A subject having a genotype of “LL/TT+” is LL and TT positive and may or may not have an additional genotype.

Serotonin refers to 5-hydroxytryptamine (5-HT).

The term “SNP” refers to a single nucleotide polymorphism in a reference gene or a reference protein encoded by the gene. Unless otherwise specified herein, the SNP may occur in the coding or noncoding portion of a reference gene. For example, the SNP may occur in the 5′ regulatory region, an exon, an intron, or a 3′ UTR corresponding to a reference gene or a reference protein encoded by the gene.

The serotonin-3 receptor (5-HT3 receptor or 5-HT3 receptor complex) refers to a subtype of the serotonin receptor, an ion channel. 5-HT3A refers to a subunit of the 5-HT3 receptor complex, and HTR3A refers to a gene that encodes the 5-HT3A subunit. 5-HT3B refers to a subunit of the 5-HT3 receptor complex, and HTR3B refers to a gene that encodes the 5-HT3B subunit. A 5-HT3 receptor functional as an ion channel may be the 5-HT3A receptor complex, composed of 5 identical 5-HT3A subunits as a 5-HT3A homopentameric protein complex. A 5-HT3 receptor functional as an ion channel may be the 5-HT3AB receptor (5-HT3AB receptor complex, 5-HT3 A/B receptor complex), composed of 5-HT3A and 5-HT3B subunits as a 5-HT3AB heteropentameric protein complex. The term “HTR3 AB complex” can refer collectively to the genes, HTR3A and HTR3B, that encode the 5-HT3A subunit and the 5-HT3B subunit, respectively, the 5-HT3A and 5-HT3B subunits forming the 5-HT3AB receptor that is a heteropentameric protein complex. For example, the phrase “SNPs [single nucleotide polymorphisms] at the HTR3 AB complex” can mean SNPs in one or both of the HTR3A and HTR3B genes. As referred to herein, a “SNP at the HTR3 AB complex” or similar language, refers to a single nucleotide polymorphism in the coding or noncoding portion of the HTR3A or HTR3B gene. For example, the SNP may occur in the 5′ regulatory region, an exon, an intron, or a 3′ UTR of the HTR3A or HTR3B gene. In certain embodiments, one or more SNPs at the HTR3 AB complex are associated with increased expression and/or sensitivity of functional HTR3A and/or HTR3B.

The serotonin transporter (also referred to as SERT, 5HTT, HTT, and OCD1) is encoded by the SLC6A4 gene.

Serotonin transporter-linked polymorphic region (5-HTTLPR) is a polymorphic region in SLC6A4, the gene that in humans encodes the serotonin transporter.

Reference to a protein can mean or can include reference to the gene that encodes that protein, and reference to a protein complex can mean or can include reference to the gene or genes that encode that protein complex, if the context so indicates. For example, reference to the “serotonin transporter” can mean or can include reference to the gene that encodes the serotonin transporter, such as SLC6A4 in humans. For example, the phrase “SNPs [single nucleotide polymorphisms] at . . . the serotonin transporter” can mean SNPs in the gene that encodes the serotonin transporter, such as SLC6A4 in humans.

Ondansetron (e.g., Zofran® and ondansetron generics) is a highly selective serotonin receptor (5-HT3) antagonist.

Alcohol use disorder (AUD) is defined by the Diagnostic and Statistical Manual of Mental Disorders (DSM-5) as “a problematic pattern of alcohol use leading to clinically significant impairment or distress”. There are 11 criteria. The range of severity of AUD includes mild, moderate and severe based solely on the number of criteria an individual meets. A number of symptoms can be associated with AUD, including the following:
1. Alcohol is often taken in larger amounts or over a longer period than was intended.
2. There is a persistent desire or unsuccessful efforts to cut down or control alcohol use.
3. A great deal of time is spent in activities necessary to obtain alcohol, use alcohol, or recover from its effects.
4. Craving, or a strong desire or urge to use alcohol.
5. Recurrent alcohol use resulting in a failure to fulfill major obligations at work, school, or home.
6. Continued alcohol use despite having persistent or recurrent social or interpersonal problems caused or exacerbated by the effects of alcohol.
7. Important social, occupational, or recreational activities are given up or reduced because of alcohol use.
8. Recurrent alcohol use in situations in which it is physically hazardous.
9. Alcohol use is continued despite knowledge of having a persistent or recurrent physical or psychological problem that is likely to have been caused or exacerbated by alcohol.
10. Tolerance, as defined by either of the following:
   a. A need for markedly increased amounts of alcohol to achieve intoxication or desired effect.
   b. A markedly diminished effect with continued use of the same amount of alcohol.
11. Withdrawal, as manifested by either of the following:
   a. Withdrawal symptoms for alcohol (e.g., tremors, sweating, elevated pulse and blood pressure, insomnia, anxiety, nausea or vomiting, seizures, and delirium tremens).
   b. Alcohol (or a related substance, such as a benzodiazepine) is taken to relieve or avoid withdrawal symptoms.
Over a 12-month period, 2-3 of these symptoms may indicate mild AUD, 4-5 of these symptoms may indicate moderate AUD, and 6 or more of these symptoms may indicate severe AUD. (www.niaaa.nih. gov/health-professionals-communities/core-resource-on-alcohol/alcohol-use-disorder-risk-diagnosis-recovery #:~:text=Alcohol%20use %20disorder %20(AUD)%20is,possible%2011%2C%20in%20the%20past).

### EMBODIMENTS

The disclosure provides methods of treating a subject having alcohol use disorder that includes administering ondansetron to a subject having a specified genotype corresponding to genetic variants that effect the 5-HT3 receptor complexes to which ondansetron binds (e.g., variants in the 5-HT3A receptor and/or 5-HT3A receptor) and/or genetic variants that effect the expression of the serotonin transporter (e.g., variants in the, 5-HTTLPR and/or 3′UTR of the SLC6A4 gene that encodes the serotonin transporter). In some embodiments, the disclosure provides a method of altering the drinking habit and/or treating alcohol dependency of a subject who is a heavy drinker. In additional embodiments, the disclosure provides a method of predicting the likelihood that a subject having alcohol use disorder who is a heavy drinker will achieve an altered drinking habit, reduced alcohol dependency, and/or decreased drinking days upon the administration of ondansetron according to the provided methods.

In some embodiments, the disclosure provides a method of treating a subject having alcohol use disorder to achieve one or more (preferably, two or more, three or more, or four or more) of the following effects (a)-(e) in one or both of the last two months of a treatment period, if the treatment period is five months or below, or in month 5 and/or month 6, or one or both of the last two months of the treatment period, if the treatment period is six months or longer:
(a) zero heavy drinking days (HHDs), wherein an HDD is a day with ethanol consumption of 60 grams or more for males and 40 grams or more for females;
(b) a reduction of percent heavy drinking days of at least 10% (for example, a reduction of at least 20%, 30%, 40%, 50%, or 60%, preferably, at least 10% in addition to placebo effect) compared to that observed for the 4 weeks prior to the treatment period (“baseline”);
(c) at least two (for example, at least 2, 3, 4, 5, 6, 7, 8, 9, 10, 11, 12, 15, 20, etc., preferably, at least two in addition to placebo effect) no alcohol drinking days (“NDDs” or “abstinent days”)
(d) a reduction of total alcohol consumption of at least 5% (for example, a reduction of at least 10%, 15%, 20%, 30%, 40%, etc., preferably, at least 5% in addition to placebo effect) compared to baseline; and
(e) a reduction of at least one (for example, a reduction of at least 2, 3, 4, 5, 6 etc., preferably, at least one in addition to placebo effect) drink per drinking day, wherein each drink contains about 10 grams of ethanol;
   the method comprising administering to the subject an effective amount of ondansetron,
   wherein the subject is characterized as having the following baseline characteristics:
(1) ≥6 HDDs;
(2) consuming less than 10 drinks per drinking day;
(3) having an average alcohol consumption at a medium risk level, which is ≥40 grams of ethanol per day for males and ≥20 grams of ethanol per day for females; and
(4)≤14 consecutive abstinent days; and the subject has a genotype of: (i) AG+, i.e., rs1150226-AG single nucleotide polymorphism (SNP) in HTR3A (“AG”) and optionally any one or more of other genotypic variations (e.g., SNP(s)) at the HTR3 AB complex or the serotonin transporter, or a genotype of GG+, i.e., rs1176713-GG SNP in HTR3A (“GG”) and optionally any one or more of other genotypic variations (e.g., SNP(s)) at the HTR3 AB complex or the non-coding or coding region of the serotonin transporter (e.g., a genotypic variation in the 5-HTT linked polymorphic region (5-HTTLPR) and/or 3′ UTR of the SLC6A4 gene that encodes the serotonin transporter); and/or (ii) the subject does not have a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter. As used herein, when a subject is referred to as not having (or the subject does not have) a genotype of LL or TT, it should be understood that the subject has neither the LL genotype nor the TT genotype, alone or in combination. Thus, such subject also does not have the LL/TT genotype, which is defined herein as having both the LL and TT genotypes.

In some embodiments, the disclosure provides a method of treating a subject having alcohol use disorder to achieve zero heavy drinking days (HHDs), wherein an HDD is a day with ethanol consumption of 60 grams or more for males and 40 grams or more for females. In further embodiments, the disclosure provides a method of treating a subject having alcohol use disorder to achieve zero HHD and one or more, two or more or three or more of the following effects (a)-(d) in one or both of the last two months of a treatment period, if the treatment period is five months or below, or in month 5 and/or month 6 if the treatment period is six months or longer:
(a) a reduction of percent heavy drinking days of at least 10% (for example, a reduction of at least 20%, 30%, 40%, 50%, or 60%, preferably, at least 10% in addition to placebo effect) compared to that observed for the 4 weeks prior to the treatment period (“baseline”);
(b) at least two (for example, at least 2, 3, 4, 5, 6, 7, 8, 9, 10, 11, 12, 15, 20, etc., preferably, at least two in addition to placebo effect) no alcohol drinking days (“NDDs” or “abstinent days”) (c) a reduction of total alcohol consumption of at least 5% (for example, a reduction of at least 10%, 15%, 20%, 30%, 40%, etc., preferably, at least 5% in addition to placebo effect) compared to baseline; and
(d) a reduction of at least one (for example, a reduction of at least 2, 3, 4, 5, 6 etc., preferably, at least one in addition to placebo effect) drink per drinking day, wherein each drink contains about 10 grams of ethanol.

In some embodiments, the disclosure provides a method of treating a subject having alcohol use disorder to achieve zero heavy drinking days (HHDs), wherein an HDD is a day with ethanol consumption of 60 grams or more for males and 40 grams or more for females and to further achieve a reduction of percent heavy drinking days of at least 10% (for example, a reduction of at least 20%, 30%, 40%, 50%, or 60%, preferably, at least 10% in addition to placebo effect) compared to that observed for the 4 weeks prior to the treatment period (“baseline”).

In some embodiments, the disclosure provides a method of treating a subject having alcohol use disorder to achieve zero heavy drinking days (HHDs), wherein an HDD is a day with ethanol consumption of 60 grams or more for males and 40 grams or more for females and to further achieve a reduction of total alcohol consumption of at least 5% (for example, a reduction of at least 10%, 15%, 20%, 30%, 40%, etc., preferably, at least 5% in addition to placebo effect) compared to baseline.

In some embodiments, the disclosure provides a method of treating a subject having alcohol use disorder to achieve a reduction of percent heavy drinking days of at least 10% (for example, a reduction of at least 20%, 30%, 40%, 50%, or 60%, preferably, at least 10% in addition to placebo effect) compared to that observed for the 4 weeks prior to the treatment period (“baseline”). In further embodiments, the disclosure provides a method of treating a subject having alcohol use disorder to achieve a reduction of percent heavy drinking days of at least 5%, preferably, at least 10%, in addition in addition to placebo effect compared to that observed for the 4 weeks prior to the treatment period (“baseline”) and one or more, two or more or three or more of the following effects (a)-(d) in one or both of the last two months of a treatment period, if the treatment period is five months or below, or in month 5 and/or month 6 if the treatment period is six months or longer:
(a) zero heavy drinking days (HHDs), wherein an HDD is a day with ethanol consumption of 60 grams or more for males and 40 grams or more for females;
(b) at least two (for example, at least 2, 3, 4, 5, 6, 7, 8, 9, 10, 11, 12, 15, 20, etc., preferably, at least two in addition to placebo effect) no alcohol drinking days (“NDDs” or “abstinent days”)
(c) a reduction of total alcohol consumption of at least 5% (for example, a reduction of at least 10%, 15%, 20%, 30%, 40%, etc., preferably, at least 5% in addition to placebo effect) compared to baseline; and
(d) a reduction of at least one (for example, a reduction of at least 2, 3, 4, 5, 6 etc., preferably, at least one in addition to placebo effect) drink per drinking day, wherein each drink contains about 10 grams of ethanol.

In some embodiments, the disclosure provides a method of treating a subject having alcohol use disorder to achieve a reduction of total alcohol consumption of at least 5% (for example, a reduction of at least 10%, 15%, 20%, 30%, 40%, etc., preferably, at least 5% in addition to placebo effect) compared to baseline zero, and to further achieve a reduction of total alcohol consumption of at least 5% (for example, a reduction of at least 10%, 15%, 20%, 30%, 40%, etc., preferably, at least 5% in addition to placebo effect) compared to baseline.

In some embodiments, the disclosure provides a method of treating a subject having alcohol use disorder to achieve a reduction of total alcohol consumption of at least 5% (for example, a reduction of at least 10%, 15%, 20%, 30%, 40%, etc., preferably, at least 5% in addition to placebo effect) compared to baseline. In further embodiments, the disclosure provides a method of treating a subject having alcohol use disorder to achieve a reduction of total alcohol consumption of at least 5% compared to baseline, and one or more, two or more or three or more of the following effects (a)-(d) in one or both of the last two months of a treatment period, if the treatment period is five months or below, or in month 5 and/or month 6 if the treatment period is six months or longer:
(a) zero heavy drinking days (HHDs), wherein an HDD is a day with ethanol consumption of 60 grams or more for males and 40 grams or more for females;
(b) a reduction of percent heavy drinking days of at least 10% (for example, a reduction of at least 20%, 30%, 40%, 50%, or 60%, preferably, at least 10% in addition to placebo effect) compared to that observed for the 4 weeks prior to the treatment period (“baseline”);
(c) at least two (for example, at least 2, 3, 4, 5, 6, 7, 8, 9, 10, 11, 12, 15, 20, etc., preferably, at least two in addition to placebo effect) no alcohol drinking days (“NDDs” or “abstinent days”);
(d) a reduction of total alcohol consumption of at least 5% (for example, a reduction of at least 10%, 15%, 20%, 30%, 40%, etc., preferably, at least 5% in addition to placebo effect) compared to baseline; and
(e) a reduction of at least one (for example, a reduction of at least 2, 3, 4, 5, 6 etc., preferably, at least one in addition to placebo effect) drink per drinking day, wherein each drink contains about 10 grams of ethanol.

In some embodiments, the disclosure provides a method of treating a subject having alcohol use disorder to achieve a reduction of total alcohol consumption of at least 5% (for example, a reduction of at least 10%, 15%, 20%, 30%, 40%, etc., preferably, at least 5% in addition to placebo effect) compared to baseline and to further achieve zero heavy drinking days (HHDs), wherein an HDD is a day with ethanol consumption of 60 grams or more for males and 40 grams or more for females.

In some embodiments, the subject treated according to the provided methods has a genotype of AG+, wherein the subject has a genotype of AG (rs1150226-AG SNP in HTR3A) and at least one other genotypic variation (e.g., SNP(s) or other genotypic variations) at the HTR3 AB complex. In some embodiments, the subject has a genotype of AG+, wherein the subject has an AG AC, AG GG or AG AC GG genotype. In some embodiments, the subject has a genotype of AG+, wherein the subject has an AG AC genotype. In some embodiments, the subject treated according to the provided methods has a genotype of AG and at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, the subject treated according to the provided methods has a genotype of AG and at least one other genotypic variant at the HTR3 AB complex, but does not have an LL (rs4795541-LL) genotype or a TT (rs1042173-TT) genotype. In some embodiments, the subject treated according to the provided methods has a genotype of AG and at least one other genotypic variant at the HTR3 AB complex, but does not have an LL TT genotype. In some embodiments, the subject treated according to the provided methods has a genotype of AG and at least one other genotypic variant at the HTR3 AB complex, but does not have a GG genotype. In some embodiments, the subject treated according to the provided methods has a genotype of AG and at least one other genotypic variant at the HTR3 AB complex, but does not have more than two, preferably, does not have more than one, more preferably, does not have any one, of the genotypes selected from GG genotype, LL (rs4795541-LL) genotype, and TT (rs1042173-TT) genotype. In some embodiments, the subject treated according to the provided methods has a genotype of AG and at least one other genotypic variant at the HTR3 AB complex, but does not have any of the genotypes selected from GG genotype, LL (rs4795541-LL) genotype, and TT (rs1042173-TT) genotype. In some embodiments, the subject treated according to the provided methods has a genotype of AG AC and optionally at least one other genotypic variant at the HTR3 AB complex, but does not have a GG genotype. In some embodiments, the subject treated according to the provided methods has a genotype of AG AC and optionally at least one other genotypic variant at the HTR3 AB complex, but does not have more than two, preferably, does not have more than one, more preferably, does not have any one, of the genotypes selected from GG genotype, LL (rs4795541-LL) genotype, and TT (rs1042173-TT) genotype. In some embodiments, the subject treated according to the provided methods has a genotype of AG AC and optionally at least one other genotypic variant at the HTR3 AB complex, but does not have any of the genotypes selected from GG genotype, LL (rs4795541-LL) genotype, and TT (rs1042173-TT) genotype.

In some embodiments, the subject has a genotype of AG+, wherein the subject has an AG genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter.

In some embodiments, the subject having a genotype of AG+ has an AG (rs1150226-AG) genotype and one or more further genotypic variations in the 5-HTR3A. In some embodiments, the subject having a genotype of AG+ has an AG and a GG (rs1176713-GG-SNP in HTR3A) genotype. In some embodiments, the subject having a genotype of AG+ does not have a GG (rs1176713-GG-SNP in HTR3A) genotype. In some embodiments, the subject has an AG and GG genotype and further contains one or more genotypic variations in the 5-HTR3A. In some embodiments, the subject has an AG genotype, one or more further genotypic variations in the 5-HTR3A, and further contains at least one genotypic variation in the non-coding and/or coding region of the serotonin transporter gene (e.g., one or more variations in the 5-HTTLPR and/or 3′ UTR of SLC6A4). In some embodiments, the subject has a genotype of AG+, wherein the subject has an AG AC LL/TT, AG GG LL/TT or AG AC GG LL/TT genotype. In some embodiments, the subject has an AG genotype and one or more further genotypic variations in the 5-HTR3A (e.g., a GG genotype), but does not have an LL TT genotype. In some embodiments, the subject has an AG genotype and one or more further genotypic variations in the 5-HTR3A (e.g., a GG genotype), but does not have an LL TT genotype. In some embodiments, the subject has an AG genotype and one or more further genotypic variations in the 5-HTR3A, but does not have more than two, preferably, does not have more than one, more preferably, does not have any one, of the genotypes selected from GG genotype, LL (rs4795541-LL) genotype, and TT (rs1042173-TT) genotype. In some embodiments, the subject has an AG genotype and one or more further genotypic variations in the 5-HTR3A, but does not have any of the genotypes selected from GG genotype, LL (rs4795541-LL) genotype, and TT (rs1042173-TT) genotype.

In some embodiments, the subject having a genotype of AG+ has an AG genotype and one or more genotypic variations in the 5-HTR3B. In some embodiments, the subject has an AG genotype and an AC (rs17614942-AC) genotype. In further embodiments the subject has an AG AC genotype and further contains one or more genotypic variations in 5-HTR3A and 5-HTR3B. In some embodiments the subject has an AG AC genotype and further contains one or more genotypic variations in the 5-HTR3A. In some embodiments the subject has an AG AC genotype and further contains one or more genotypic variations in the 5-HTR3B. In some embodiments, the subject treated according to the provided methods has a genotype of AG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, the subject has an AG genotype and one or more further genotypic variations in the 5-HTR3A (e.g., a GG genotype), but does not have an LL genotype or a TT genotype. In some embodiments, the subject has an AG genotype and at least one genotypic variation in the 5-HTR3A (e.g., a GG genotype), but does not have an LL TT genotype. In some embodiments, the subject having a genotype of AG+ has an AG genotype and one or more genotypic variations in the 5-HTR3B but does not have more than two, preferably, does not have more than one, more preferably, does not have any one, of the genotypes selected from GG genotype, LL (rs4795541-LL) genotype, and TT (rs1042173-TT) genotype. In some embodiments, the subject having a genotype of AG+ has an AG genotype and one or more genotypic variations in the 5-HTR3B but does not have any of the genotypes selected from GG genotype, LL (rs4795541-LL) genotype, and TT (rs1042173-TT) genotype.

In some embodiments, the subject having a genotype of AG+ has an AG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, the subject has an AG genotype and further contains a genotypic variation in the non-coding region of the SLC6A4 gene that encodes the serotonin transporter. In some embodiments, the subject has an AG genotype and further contains a genotypic variation in the 5-HTTLPR. In further embodiments, the subject has an AG genotype and further contains an LL (rs4795541-LL) genotype. In other embodiments, the subject has an AG genotype and further contains one or more genotypic variations in the 5-HTTLPR, but does not have an LL genotype. In some embodiments, the subject has an AG genotype and further contains a genotypic variation in the 3′ UTR of SLC6A4. In further embodiments, the subject has an AG genotype and contains a TT (rs1042173-TT) genotype. In further embodiments, the subject has an AG genotype and contains a TT (rs1042173-TT) genotype. In other embodiments, the subject has an AG genotype and further contains a genotypic variation in the 3′ UTR of SLC6A4, but does not have a TT genotype. In some embodiments, the subject has an AG genotype and further contains a genotypic variation that alters the splicing, transcription and/or translation of SLC6A4 nucleic acids. In some embodiments, the subject has an AG genotype and further contains a genotypic variation in the coding region of the SLC6A4 gene that encodes the serotonin transporter. In some embodiments, the subject having a genotype of AG+ has an AG genotype and one or more genotypic variations in the non-coding and/or coding region of the serotonin transporter gene but does not have an LL genotype or a TT genotype. In some embodiments, the subject has an AG genotype and at least one genotypic variation in the 5-HTR3A (e.g., a GG genotype), but does not have an LL TT genotype. In some embodiments, the subject having a genotype of AG+ has an AG genotype and one or more genotypic variations in the non-coding and/or coding region of the serotonin transporter gene but does not have more than two, preferably, does not have more than one, more preferably, does not have any one, of the genotypes selected from GG genotype, LL (rs4795541-LL) genotype, and TT (rs1042173-TT) genotype. In some embodiments, the subject having a genotype of AG+ has an AG genotype and one or more genotypic variations in the non-coding and/or coding region of the serotonin transporter gene but does not have any of the genotypes selected from GG genotype, LL (rs4795541-LL) genotype, and TT (rs1042173-TT) genotype.

In some embodiments, the subject treated according to the provided methods has a genotype of GG+, wherein the subject has a genotype of GG (rs1176713-GG-SNP in HTR3A)) and at least one other genotypic variation (e.g., SNP(s) or other genotypic variations) at the HTR3 AB complex. In some embodiments, the subject has a genotype of GG+, wherein the subject has an GG AC LL/TT, GG AG LL/TT or GG AC AG LL/TT genotype. In some embodiments, the subject treated according to the provided methods has a genotype of AG and at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, the subject treated according to the provided methods has a genotype of AG and at least one other genotypic variant at the HTR3 AB complex, but does not have an LL (rs4795541-LL) genotype or a TT (rs1042173-TT) genotype. In some embodiments, the subject treated according to the provided methods has a genotype of AG and at least one other genotypic variant at the HTR3 AB complex, but does not have an LL TT genotype.

In some embodiments, the subject has a genotype of GG+, wherein the subject has an GG (rs1176713-GG-SNP in HTR3A) genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter.

In some embodiments, the subject having a genotype of GG+ has a GG (rs1176713-GG) genotype and one or more further genotypic variations in the 5-HTR3A. In some embodiments, the subject having a genotype of GG+ has a GG and an AG (rs1150226-AG) genotype. In some embodiments, the subject has a GG and AG genotype and further contains one or more genotypic variations in the 5-HTR3A. In some embodiments, the subject has a GG genotype, one or more further genotypic variations in the 5-HTR3A, and further contains at least one genotypic variation in the non-coding and/or coding region of the serotonin transporter gene (e.g., one or more variations in the 5-HTTLPR and/or 3′ UTR of SLC6A4). In some embodiments, the subject has a genotype of GG+, wherein the subject has a GG AC LL/TT, GG AG LL/TT or GG AC AG LL/TT genotype. In some embodiments, the subject has a GG genotype and one or more further genotypic variations in the 5-HTR3A (e.g., an AG genotype), but does not have an LL genotype or a TT genotype. In some embodiments, the subject has a GG genotype and one or more further genotypic variations in the 5-HTR3A (e.g., an AG genotype), but does not have an LL TT genotype.

In some embodiments, the subject having a genotype of GG+ has a GG genotype and one or more genotypic variations in the 5-HTR3B. In some embodiments, the subject has a GG genotype and an AC (rs17614942-AC) genotype. In further embodiments the subject has an GG AC genotype and further contains one or more genotypic variations in 5-HTR3A and 5-HTR3B. In some embodiments the subject has a GG AC genotype and further contains one or more genotypic variations in the 5-HTR3A. In some embodiments the subject has an GG AC genotype and further contains one or more genotypic variations in the 5-HTR3B. In some embodiments, the subject treated according to the provided methods has a genotype of GG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, the subject has a GG genotype and one or more further genotypic variations in the 5-HTR3A (e.g., an AG genotype), but does not have an LL genotype or a TT genotype. In some embodiments, the subject has a GG genotype and at least one genotypic variation in the 5-HTR3A (e.g., an AG genotype), but does not have an LL TT genotype.

In some embodiments, the subject having a genotype of GG+ has a GG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, the subject has a GG genotype and further contains a genotypic variation in the non-coding region of the SLC6A4 gene that encodes the serotonin transporter. In some embodiments, the subject has a GG genotype and further contains a genotypic variation in the 5-HTTLPR. In further embodiments, the subject has a GG genotype and further contains an LL (rs4795541-LL) genotype. In other embodiments, the subject has a GG genotype and further contains one or more genotypic variations in the 5-HTTLPR, but does not have an LL genotype. In some embodiments, the subject has a GG genotype and further contains a genotypic variation in the 3′ UTR of SLC6A4. In further embodiments, the subject has a GG genotype and contains a TT (rs1042173-TT) genotype. In further embodiments, the subject has a GG genotype and contains a TT (rs1042173-TT) genotype. In other embodiments, the subject has a GG genotype and further contains a genotypic variation in the 3′ UTR of SLC6A4, but does not have a TT genotype. In some embodiments, the subject has a GG genotype and further contains a genotypic variation that alters the splicing, transcription and/or translation of SLC6A4 nucleic acids. In some embodiments, the subject has a GG genotype and further contains a genotypic variation in the coding region of the SLC6A4 gene that encodes the serotonin transporter. In some embodiments, the subject having a genotype of GG+ has a GG genotype and one or more genotypic variations in the non-coding and/or coding region of the serotonin transporter gene but does not have an LL genotype or a TT genotype. In some embodiments, the subject has a GG genotype and at least one genotypic variation in the 5-HTR3A (e.g., an AG genotype), but does not have an LL TT genotype.

In some embodiments, the subject treated according to the provided methods has at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but does not have more than two, preferably, does not have more than one, more preferably, does not have any one, of the genotypes selected from GG genotype, LL (rs4795541-LL) genotype, and TT (rs1042173-TT) genotype. In some embodiments, the subject treated according to the provided methods has at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but does not have a GG genotype. In some embodiments, the subject treated according to the provided methods has at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but does not have an LL genotype. In some embodiments, the subject treated according to the provided methods has at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but does not have a TT genotype. In some embodiments, the subject treated according to the provided methods has at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but does not have a LL and TT genotype. In some embodiments, the subject treated according to the provided methods has at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but does not have any of the genotypes selected from GG genotype, LL (rs4795541-LL) genotype, and TT (rs1042173-TT) genotype.

In some embodiments, the subject treated according to the provided methods has mild AUD at baseline. In some embodiments, the subject treated according to the provided methods has moderate AUD at baseline. In some embodiments, the subject treated according to the provided methods has severe AUD at baseline. In some embodiments, the provided methods are effective in converting the subject's severe AUD into a moderate or mild AUD. In some embodiments, the provided methods are effective in reducing the subject's craving for alcohol.

In some embodiments, the ondansetron is administered to the subject daily in an amount ranging from about 0.20 mg to about 1.5 mg. In some embodiments, the ondansetron is administered to the subject daily in an amount ranging from about 0.25 mg to about 1 mg. In some embodiments, the ondansetron is administered to the subject daily in an amount ranging from about 0.4 mg to about 0.8 mg. In some embodiments, the ondansetron is administered to the subject twice a day (b.i.d.), with each dose of about 0.33 mg.

In some embodiments, the treatment period during which the subject is administered the ondansetron is at least six months. In some embodiments the treatment period is at least 9 months, at least 12 months, or at least 24 months. In some embodiments, the treatment period is 6-12 months, 9-12 months, or 1-2 years.

In some embodiments, ondansetron is administered to the subject such that the subject has less than 10, 9, 8, 7, 6, 5, 4, 3, 2, or 1, or zero heavy drinking days (HDD) in month 6 of the treatment period. In some embodiments, ondansetron is administered to the subject such that the subject has less than 5 HDD in month 6 of the treatment period. In some embodiments, ondansetron is administered to the subject such that the subject has zero HDD in month 6 of the treatment period.

In some embodiments, the ondansetron is administered to the subject such that the subject has a reduction of percent heavy drinking days in months 5 and/or 6 of the treatment period by at least 30%, 25%, 20%, 15%, 10%, 7%, or 5% in addition to the placebo effect, compared to baseline. In some embodiments, the ondansetron is administered to the subject such that the subject has a reduction of percent heavy drinking days in months 5 and/or 6 of the treatment period by at least 5%, preferably, at least 10% or at least 12%, in addition to the placebo effect, compared to baseline. In some embodiments, the ondansetron is administered to the subject such that the subject has a reduction of percent heavy drinking days in months 5 and/or 6 of the treatment period by reduction of 10-40%, 10-30% or 10-20% in addition to the placebo effect, compared to baseline. In some embodiments, the ondansetron is administered to the subject such that the subject has a reduction of percent heavy drinking days in months 5 and/or 6 of the treatment period by reduction of 15-35%, 15-30%, 15-25%, or 15-20% in addition to the placebo effect, compared to baseline.

In some embodiments, ondansetron is administered to the subject such that the subject has 2-15, 2-10, or 2-5 days of no drinking days (i.e., no drinking alcohol (abstinence)) in month 6 of the treatment period during which the subject is administered the ondansetron. In further embodiments, ondansetron is administered to the subject such that the subject has 2-4 more days of no drinking days in addition to placebo effect in month 6 of the treatment period.

In some embodiments, ondansetron is administered to the subject such that the subject has a reduction of total alcohol consumption of 5-40%, 5-35%, or 7-30% in addition to the placebo effect, compared to baseline in month 6 of the treatment period. In some embodiments, ondansetron is administered to the subject such that the subject has a reduction of total alcohol consumption of 7-30% in addition to the placebo effect, compared to baseline in month 6 of the treatment period. In some embodiments, ondansetron is administered to the subject such that the subject has a reduction of total alcohol consumption by at least 5% in addition to the placebo effect, compared to baseline in month 6 of the treatment period.

In some embodiments, ondansetron is administered to the subject such that the subject has a reduction of 1-3 drinks per drinking day in addition to the placebo effect, compared to baseline in month 6 of the treatment period.

In another embodiment, the disclosure provides a method of treating alcohol use disorder, wherein the method comprises:
administering ondansetron to a subject in need of treatment who belongs to a patient population characterized as (i) being heavy drinkers; and
(ii) having a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (iii) not having a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
wherein the ondansetron is administered to the subject with a dosing regimen that is effective in treating the patient population such that the percentage of the patient population having zero HDD (PNHDD) in one or both of the last two months of a treatment period, if the treatment period is five months or below, or in month 5 and/or month 6, or one or both of the last two months of the treatment period, if the treatment period is six months or longer, is higher than that of a placebo-controlled patient population, e.g., 25-50%, 30-40%, or 30-35% higher than that of a placebo-controlled patient population,
wherein an HDD is a day with ethanol consumption of 60 grams or more for males and 40 grams or more for females, and
wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days. For example, in some embodiments, the ondansetron is administered to the subject with a dosing regimen that is effective in treating the patient population such that the percentage of the patient population having zero HDD (PNHDD) in one or both of the last two months of a treatment period, if the treatment period is five months or below, or in month 5 and/or month 6, or one or both of the last two months of the treatment period, if the treatment period is six months or longer, is at least 10%, 20%, 30%, 40%, or 50% higher than that of the placebo-controlled patient population, i.e., the percentage of the patient population having zero HDD minus the percentage of the patient population having zero HDD of the placebo-controlled patient population is at least 10%, 20%, 30%, 40%, or 50%, in the relevant period of time. For example, in some embodiments, the ondansetron is administered to the subject with a dosing regimen that is effective in treating the patient population such that the percentage of the patient population having zero HDD (PNHDD) in one or both of the last two months of a treatment period, if the treatment period is five months or below, or in month 5 and/or month 6, or one or both of the last two months of the treatment period, if the treatment period is six months or longer, is 25-50%, 30-40%, or 30-35% higher than that of the placebo-controlled patient population, i.e., the percentage of the patient population having zero HDD minus the percentage of the patient population having zero HDD of the placebo-controlled patient population is 25-50%, 30-40%, or 30-35%, in the relevant period of time.

In some embodiments, all the subjects of the patient population have a genotype of AG+, wherein all the subjects of the patient population have a genotype of AG (rs1150226-AG SNP in HTR3A) and at least one other genotypic variation (e.g., SNP(s) or other genotypic variations) at the HTR3 AB complex. In some embodiments, all the subjects of the patient population have a genotype of AG+, wherein all the subjects in the patient population have an AG AC, AG GG or AG AC GG genotype. In some embodiments, all the subjects of the patient population have an AG AC genotype. In some embodiments, all the subjects of the patient population have a genotype of AG, at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, all the subjects of the patient population have a genotype of AG and at least one other genotypic variant at the HTR3 AB complex, but do not have an LL (rs4795541-LL) genotype or a TT (rs1042173-TT) genotype. In some embodiments, all the subjects of the patient population have a genotype of AG and at least one other genotypic variant at the HTR3 AB complex, but do not have an LL TT genotype. In some embodiments, all the subjects of the patient population have a genotype of AG+, wherein all the subjects of the patient population have an AG genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter. In some embodiments, all the subjects of the patient population have a genotype of AG and at least one other genotypic variant at the HTR3 AB complex, but do not have a GG genotype. In some embodiments, all the subjects of the patient population have a genotype of AG and at least one other genotypic variant at the HTR3 AB complex, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have a genotype of AG and at least one other genotypic variant at the HTR3 AB complex, but do not have any of the genotypes selected from GG genotype, LL (rs4795541-LL) genotype, and TT (rs1042173-TT) genotype. In some embodiments, all the subjects of the patient population have a genotype of AG AC and optionally at least one other genotypic variant at the HTR3 AB complex, but do not have a GG genotype. In some embodiments, all the subjects of the patient population have a genotype of AG AC and optionally at least one other genotypic variant at the HTR3 AB complex, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have a genotype of AG AC and optionally at least one other genotypic variant at the HTR3 AB complex, but do not have any of the genotypes selected from GG genotype, LL (rs4795541-LL) genotype, and TT (rs1042173-TT) genotype.

In some embodiments, all the subjects of the patient population have an AG+ genotype wherein all the subjects of the patient population have an AG (rs1150226-AG) genotype and one or more further genotypic variations in the 5-HTR3A. In some embodiments, all the subjects of the patient population have an AG genotype and a GG (rs1176713-GG-SNP in HTR3A) genotype. In some embodiments, all the subjects of the patient population have an AG genotype but do not have a GG (rs1176713-GG-SNP in HTR3A) genotype. In some embodiments, all the subjects of the patient population have an AG GG genotype and further have one or more genotypic variations in the 5-HTR3A. In some embodiments, all the subjects of the patient population have an AG genotype, one or more further genotypic variations in the 5-HTR3A, and further have at least one genotypic variation in the non-coding and/or coding region of the serotonin transporter gene (e.g., one or more variations in the 5-HTTLPR and/or 3′ UTR of SLC6A4). In some embodiments, all the subjects of the patient population have a genotype of AG+, wherein all the subjects in the patient population have an AG AC LL/TT, AG GG LL/TT or AG AC GG LL/TT genotype. In some embodiments, all the subjects of the patient population have an AG genotype and one or more further genotypic variations in the 5-HTR3A (e.g., a GG genotype), but do not have an LL genotype or a TT genotype. In some embodiments, all the subjects of the patient population have AG genotype and one or more further genotypic variations in the 5-HTR3A (e.g., a GG genotype), but do not have a LL TT genotype. In some embodiments, all the subjects of the patient population have an AG genotype and one or more further genotypic variations in the 5-HTR3A, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more further genotypic variations in the 5-HTR3A, but do not have any of the genotypes selected from GG genotype, LL (rs4795541-LL) genotype, and TT (rs1042173-TT) genotype.

In some embodiments, all the subjects of the patient population have a genotype of AG+ wherein all the subjects of the patient population have an AG genotype and one or more genotypic variations in the 5-HTR3B. In some embodiments, all the subjects of the patient population have an AG genotype and an AC (rs17614942-AC) genotype. In further embodiments all the subjects of the patient population have an AG AC genotype and further have one or more genotypic variations in 5-HTR3A and 5-HTR3B. In some embodiments all the subjects of the patient population have an AG AC genotype and further have one or more genotypic variations in the 5-HTR3A. In some embodiments all the subjects of the patient population have an AG AC genotype and further have one or more genotypic variations in the 5-HTR3B. In some embodiments, all the subjects of the patient population have a genotype of AG, at least one genotypic variant in the 5-HTR3B (e.g., AC) and at least one genotypic variant in the non-coding and/or coding region of the serotonin transporter gene (e.g., one or more variations in the 5-HTTLPR and/or 3-untranslated region of SLC6A4 gene that encodes the serotonin transporter). In some embodiments, all the subjects of the patient population have an AG genotype and one or more further genotypic variations in the 5-HTR3A (e.g., a GG genotype), but do not have an LL genotype or a TT genotype. In some embodiments, all the subjects of the patient population have an AG genotype and at least one genotypic variation in the 5-HTR3A (e.g., a GG genotype), but do not have an LL TT genotype. In some embodiments, all the subjects of the patient population have an AG genotype and one or more genotypic variations in the 5-HTR3B but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more genotypic variations in the 5-HTR3B but do not have any of the genotypes selected from GG genotype, LL (rs4795541-LL) genotype, and TT (rs1042173-TT) genotype.

In some embodiments, all the subjects of the patient population have a genotype of AG+, wherein all the patients in the population have an AG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, all the subjects of the patient population have an AG genotype and further have a genotypic variation in the non-coding region of the SLC6A4 gene that encodes the serotonin transporter. In some embodiments, all the subjects of the patient population have an AG genotype and further have a genotypic variation in the 5-HTTLPR. In further embodiments, all the subjects of the patient population have an AG genotype and further have an LL (rs4795541-LL) genotype. In other embodiments, all the subjects of the patient population have an AG genotype and further have one or more genotypic variations in the 5-HTTLPR, but do not have an LL genotype. In some embodiments, all the subjects of the patient population have an AG genotype and further have a genotypic variation in the 3′ UTR of SLC6A4. In further embodiments, all the subjects of the patient population have an AG genotype and have a TT (rs1042173-TT) genotype. In further embodiments, all the subjects of the patient population have an AG genotype and have a TT (rs1042173-TT) genotype. In other embodiments, all the subjects of the patient population have an AG genotype and further have a genotypic variation in the 3′ UTR of SLC6A4, but does not have a TT genotype. In some embodiments, all the subjects of the patient population have an AG genotype and further have a genotypic variation that alters the splicing, transcription and/or translation of SLC6A4 nucleic acids. In some embodiments, all the subjects of the patient population have an AG genotype and further have a genotypic variation in the coding region of the SLC6A4 gene that encodes the serotonin transporter. In some embodiments, all the subjects of the patient population have an AG genotype and one or more genotypic variations in the non-coding and/or coding region of the serotonin transporter gene but do not have an LL genotype or a TT genotype. In some embodiments, all the subjects of the patient population have an AG genotype and at least one genotypic variation in the 5-HTR3A (e.g., a GG genotype), but do not have an LL TT genotype. In some embodiments, all the subjects of the patient population have an AG genotype and one or more genotypic variations in the non-coding and/or coding region of the serotonin transporter gene but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more genotypic variations in the non-coding and/or coding region of the serotonin transporter gene but do not have any of the genotypes selected from GG genotype, LL (rs4795541-LL) genotype, and TT (rs1042173-TT) genotype.

In some embodiments, all the subjects of the patient population treated according to the provided methods have a genotype of GG+, wherein all the subjects of the patient population have a genotype of GG (rs1176713-GG-SNP in HTR3A)) and at least one other genotypic variation (e.g., SNP(s) or other genotypic variations) at the HTR3 AB complex. In some embodiments, all the subjects of the patient population have a genotype of GG+, wherein all the subjects in the patient population have a GG AC, GG AG or GG AC AG genotype. In some embodiments, all the subjects of the patient population have an AG genotype, at least one other genotypic variant at the HTR3 AB complex, and at least one genotypic variation in the non-coding and/or coding region of the serotonin transporter gene (e.g., one or more genotypic variations in the 5-HTTLPR and/or 3′ UTR of SLC6A4). In some embodiments, all the subjects of the patient population have an AG genotype and at least one other genotypic variant at the HTR3 AB complex, but do not have an LL (rs4795541-LL) genotype or a TT (rs1042173-TT) genotype. In some embodiments, all the subjects of the patient population have an AG genotype and at least one other genotypic variant at the HTR3 AB complex, but do not have an LL TT genotype.

In some embodiments, all the subjects of the patient population have a genotype of GG+, wherein all the subjects of the patient population have a GG (rs1176713-GG-SNP in HTR3A) genotype and one or more further genotypic variations (e.g., SNP(s) or other genotypic variations) in the 5-HTR3A receptor, one or more genotypic variations in the 5-HTR3B receptor, and/or one or more genotypic variation in the 5-HTTLPR or the 3′ UTR of the SLC6A4 gene.

In some embodiments, all the subjects of the patient population have a genotype of GG+, wherein all the subjects in the patient population have an GG (rs1176713-GG) genotype and one or more further genotypic variations in the 5-HTR3A. In some embodiments, all the subjects of the patient population have a genotype of GG+ has an GG and an AG (rs1150226-AG) genotype. In some embodiments, all the subjects of the patient population have a genotype of GG+, wherein all the subjects in the patient population have an GG AC LL/TT, GG AG LL/TT or GG AC AG LL/TT genotype. In some embodiments, all the subjects of the patient population have a GG and AG genotype and further have one or more genotypic variations in the 5-HTR3A. In some embodiments, all the subjects of the patient population have a GG genotype, one or more further genotypic variations in the 5-HTR3A, and further have at least one genotypic variation in the non-coding and/or coding region of the serotonin transporter gene (e.g., one or more variations in the 5-HTTLPR and/or 3′ UTR of SLC6A4). In some embodiments, all the subjects of the patient population have GG genotype and one or more further genotypic variations in the 5-HTR3A (e.g., an AG genotype), but do not have an LL genotype or a TT genotype. In some embodiments, all the subjects of the patient population have a GG genotype and one or more further genotypic variations in the 5-HTR3A (e.g., an AG genotype), but do not have an LL TT genotype.

In some embodiments, all the subjects of the patient population have a genotype of GG+ wherein all the subjects of the patient population have GG genotype and one or more genotypic variations in the 5-HTR3B. In some embodiments, all the subjects of the patient population have a GG genotype and an AC (rs17614942-AC) genotype. In further embodiments all the subjects of the patient population have a GG AC genotype and further have one or more genotypic variations in 5-HTR3A and 5-HTR3B. In some embodiments all the subjects of the patient population have a GG AC genotype and further have one or more genotypic variations in the 5-HTR3A. In some embodiments all the subjects of the patient population have an GG AC genotype and further have one or more genotypic variations in the 5-HTR3B. In some embodiments, all the subjects of the patient population have a genotype of GG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, all the subjects of the patient population have GG genotype and one or more further genotypic variations in the 5-HTR3A (e.g., an AG genotype), but do not have an LL genotype or a TT genotype. In some embodiments, all the subjects of the patient population have a GG genotype and at least one genotypic variation in the 5-HTR3A (e.g., an AG genotype), but do not have an LL TT genotype.

In some embodiments, all the subjects of the patient population have a genotype of GG+, wherein all the subjects of the patient population have a GG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, all the subjects of the patient population have a GG genotype and further have a genotypic variation in the non-coding region of the SLC6A4 gene that encodes the serotonin transporter. In some embodiments, all the subjects of the patient population have a GG genotype and further have a genotypic variation in the 5-HTTLPR. In further embodiments, all the subjects of the patient population have a GG genotype and further have an LL (rs4795541-LL) genotype. In other embodiments, all the subjects of the patient population have a GG genotype and further have one or more genotypic variations in the 5-HTTLPR, but do not have an LL genotype. In some embodiments, all the subjects of the patient population have a GG genotype and further have a genotypic variation in the 3′ UTR of SLC6A4. In further embodiments, all the subjects of the patient population have a GG genotype and have a TT (rs1042173-TT) genotype. In further embodiments, all the subjects of the patient population have a GG genotype and have a TT (rs1042173-TT) genotype. In other embodiments, all the subjects of the patient population have a GG genotype and further have a genotypic variation in the 3′ UTR of SLC6A4, but do not have a TT genotype. In some embodiments, all the subjects of the patient population have a GG genotype and further have a genotypic variation that alters the splicing, transcription and/or translation of SLC6A4 nucleic acids. In some embodiments, all the subjects of the patient population have a GG genotype and further have a genotypic variation in the coding region of the SLC6A4. In some embodiments, all the subjects of the patient population have a GG genotype and one or more genotypic variations in the non-coding and/or coding region of the serotonin transporter gene but does not have an LL genotype or a TT genotype. In some embodiments, all the subjects of the patient population have GG genotype and at least one genotypic variation in the 5-HTR3A (e.g., an AG genotype), but do not have an LL TT genotype.

In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a GG genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have an LL genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a TT genotype. In some embodiments all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a LL and TT genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have any of the genotypes selected from GG genotype, LL (rs4795541-LL) genotype, and TT (rs1042173-TT) genotype.

In some embodiments, all the subjects of the patient population have mild AUD at baseline. In some embodiments, all the subjects of the patient population have moderate AUD at baseline. In some embodiments, all the subjects of the patient population have severe AUD at baseline. In some embodiments, the provided methods are effective in converting severe AUD into a moderate or mild AUD for the subjects of the patient population. In some embodiments, the provided methods are effective in reducing craving for alcohol for the subjects of the patient population.

In some embodiments, the treatment period is at least six months, e.g., at least 9 months, at least 12 months, at least 24 months, such as 6-12 months, or 9-12 months. In some embodiments, the treatment period during which the subject is administered the ondansetron is at least six months. In some embodiments the treatment period is at least 9 months, at least 12 months, or at least 24 months. In further embodiments, the treatment period is 6-12 months, 9-12 months, or 1-2 years.

In some embodiments, the dosing regimen is effective in treating the patient population to achieve a reduction of percent heavy drinking days (PHDD) in months 5 and/or 6 of the treatment period compared to baseline, wherein the reduction of PHDD in the patient population is statistically significantly higher than that of the placebo-controlled patient population or a patient population having only a single SNP selected from AC alone, AG alone, or GG alone.

In some embodiments, the dosing regimen is effective in treating the patient population to achieve 2-15, 2-10, 2-5 more days of no drinking days (NDD) in months 5 and/or 6 of the treatment period, than that of the placebo-controlled patient population. In some embodiments, the dosing regimen is effective in treating the patient population to achieve 2-15, 2-10, 2-5 or 2-4 more days of no drinking days (NDD) in months 5 and/or 6 of the treatment period, than that of the placebo-controlled patient population.

In some embodiments, the dosing regimen is effective in treating the patient population to achieve a reduction of total alcohol consumption (TAC) in months 5 and/or 6 of the treatment period, wherein the reduction of TAC in the patient population is higher than that of the placebo-controlled patient population.

In some embodiments, the dosing regimen is effective in treating the patient population to achieve a reduction of drinks per drinking day (DDD) in months 5 and/or 6 of the treatment period, wherein the reduction of DDD in the patient population is higher than that of the placebo-controlled patient population.

In some embodiments, when compared to the placebo-controlled patient population, the dosing regimen is effective in treating the patient population to achieve 1, 2, 3, or each of (a) a reduction of percent heavy drinking days in months 5 and/or 6 of the treatment period compared to baseline by at least 5%, 10% or 12% greater, such as 5-50%, 10-40%, or 15-35% greater; (b) 2-15, 2-10, 2-5 or 2-4 more days of no drinking days in month 6 of the treatment period; (c) a reduction of total alcohol consumption in month 6 of the treatment period compared to baseline by 5-40%, 5-35%, or 7-30% greater; and (d) a reduction of 1-3 more drinks per drinking day in month 6 of the treatment period compared to baseline.

In some embodiments, when compared to the placebo-controlled patient population, the dosing regimen is effective in treating the patient population to achieve (a) a reduction of percent heavy drinking days in months 5 and/or 6 of the treatment period compared to baseline by at least 5%, 10% or 12% greater, such as 5-50%, 10-40%, or 15-35% greater; (b) 2-15, 2-10, 2-5 or 2-4 more days of no drinking days in month 6 of the treatment period; (c) a reduction of total alcohol consumption in month 6 of the treatment period compared to baseline by 5-40%, 5-35%, or 7-30% greater; and (d) a reduction of 1-3 more drinks per drinking day in month 6 of the treatment period compared to baseline.

In some embodiments, when compared to the placebo-controlled patient population, the dosing regimen is effective in treating the patient population to achieve 1, 2, 3, or each of (a) a reduction of percent heavy drinking days in months 5 and/or 6 of the treatment period compared to baseline by at least 12% greater, such as 15-35% greater; (b) 2-4 more days of no drinking days in month 6 of the treatment period; (c) a reduction of total alcohol consumption in month 6 of the treatment period compared to baseline by 7-30% greater; and (d) a reduction of 1-3 more drinks per drinking day in month 6 of the treatment period compared to baseline.

In some embodiments, when compared to the placebo-controlled patient population, the dosing regimen is effective in treating the patient population to achieve (a) a reduction of percent heavy drinking days in months 5 and/or 6 of the treatment period compared to baseline by at least 12% greater, such as 15-35% greater; (b) 2-4 more days of no drinking days in month 6 of the treatment period; (c) a reduction of total alcohol consumption in month 6 of the treatment period compared to baseline by 7-30% greater; and (d) a reduction of 1-3 more drinks per drinking day in month 6 of the treatment period compared to baseline.

In some embodiments, during the treatment period, the ondansetron is administered to the subject daily in an amount ranging from about 0.20 mg to about 1.5 mg. In some embodiments, the ondansetron is administered to the subject daily in an amount ranging from about 0.25 mg to about 1 mg. In some embodiments, the ondansetron is administered to the subject daily in an amount ranging from about 0.4 mg to about 0.8 mg. In some embodiments, the ondansetron is administered to the subject twice a day (b.i.d.), with each dose of about 0.33 mg.

In further embodiments, the provided methods further comprise determining or having determined the subject as belonging to the patient population.

In additional embodiments, the disclosure provides a method of increasing the likelihood of achieving zero heavy drinking days (HDDs) for a subject in one or both of the last two months of a treatment period, if the treatment period is five months or below, or in month 5 and/or month 6, or one or both of the last two months of the treatment period, if the treatment period is six months or longer, wherein an HDD is a day with ethanol consumption of 60 grams or more for males and 40 grams or more for females, the method comprising:
administering ondansetron to a subject who belongs to a patient population characterized as (i) being heavy drinkers; and
(ii) having a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (iii) not having a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
wherein the ondansetron is administered to the subject with a dosing regimen that is effective in treating the patient population such that the percentage of the patient population having zero HDD (PNHDD) in one or both of the last two months of the treatment period, if the treatment period is five months or below, or in month 5 and/or month 6, or one or both of the last two months of the treatment period, if the treatment period is six months or longer, is higher than that of a placebo-controlled patient population, for example, at least 10%, 20%, 30%, 40%, or 50% higher than that of the placebo-controlled patient population, e.g., 25-50%, 30-40%, or 30-35% higher than that of the placebo-controlled patient population;
wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days.

In some embodiments, all the subjects of the patient population have a genotype of AG+, wherein all the subjects of the patient population have a genotype of AG (rs1150226-AG SNP in HTR3A) and at least one other genotypic variation (e.g., SNP(s) or other genotypic variations) at the HTR3 AB complex. In some embodiments, all the subjects of the patient population have a genotype of AG+, wherein all the subjects in the patient population have an AG AC, AG GG or AG AC GG genotype. In some embodiments, all the subjects of the patient population have an AG AC genotype. In some embodiments, all the subjects of the patient population have a genotype of AG, at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, all the subjects of the patient population have a genotype of AG and at least one other genotypic variant at the HTR3 AB complex, but do not have an LL (rs4795541-LL) genotype or a TT (rs1042173-TT) genotype. In some embodiments, all the subjects of the patient population have a genotype of AG and at least one other genotypic variant at the HTR3 AB complex, but do not have an LL TT genotype. In some embodiments, all the subjects of the patient population have a genotype of AG+, wherein all the subjects of the patient population have an AG genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter. In some embodiments, all the subjects of the patient population have a genotype of AG and at least one other genotypic variant at the HTR3 AB complex, but do not have a GG genotype. In some embodiments, all the subjects of the patient population have a genotype of AG and at least one other genotypic variant at the HTR3 AB complex, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have a genotype of AG and at least one other genotypic variant at the HTR3 AB complex, but do not have any of the genotypes selected from GG genotype, LL (rs4795541-LL) genotype, and TT (rs1042173-TT) genotype. In some embodiments, all the subjects of the patient population have a genotype of AG AC and optionally at least one other genotypic variant at the HTR3 AB complex, but do not have a GG genotype. In some embodiments, all the subjects of the patient population have a genotype of AG AC and optionally at least one other genotypic variant at the HTR3 AB complex, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have a genotype of AG AC and optionally at least one other genotypic variant at the HTR3 AB complex, but do not have any of the genotypes selected from GG genotype, LL (rs4795541-LL) genotype, and TT (rs1042173-TT) genotype.

In some embodiments, all the subjects of the patient population have an AG+ genotype wherein all the subjects of the patient population have an AG (rs1150226-AG) genotype and one or more further genotypic variations in the 5-HTR3A. In some embodiments, all the subjects of the patient population have an AG genotype and a GG (rs1176713-GG-SNP in HTR3A) genotype. In some embodiments, all the subjects of the patient population have an AG genotype but do not have a GG (rs1176713-GG-SNP in HTR3A) genotype. In some embodiments, all the subjects of the patient population have an AG GG genotype and further have one or more genotypic variations in the 5-HTR3A. In some embodiments, all the subjects of the patient population have an AG genotype, one or more further genotypic variations in the 5-HTR3A, and further have at least one genotypic variation in the non-coding and/or coding region of the serotonin transporter gene (e.g., one or more variations in the 5-HTTLPR and/or 3′ UTR of SLC6A4). In some embodiments, all the subjects of the patient population have an AG genotype and one or more further genotypic variations in the 5-HTR3A (e.g., a GG genotype), but do not have an LL genotype or a TT genotype. In some embodiments, all the subjects of the patient population have a genotype of AG+, wherein all the subjects in the patient population have an AG AC LL/TT, AG GG LL/TT or AG AC GG LL/TT genotype In some embodiments, all the subjects of the patient population have AG genotype and one or more further genotypic variations in the 5-HTR3A (e.g., a GG genotype), but do not have a LL TT genotype. In some embodiments, all the subjects of the patient population have an AG genotype and one or more further genotypic variations in the 5-HTR3A, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more further genotypic variations in the 5-HTR3A, but do not have any of the genotypes selected from GG genotype, LL (rs4795541-LL) genotype, and TT (rs1042173-TT) genotype.

In some embodiments, all the subjects of the patient population have a genotype of AG+ wherein all the subjects of the patient population have AG genotype and one or more genotypic variations in the 5-HTR3B. In some embodiments, all the subjects of the patient population have an AG genotype and an AC (rs17614942-AC) genotype. In further embodiments all the subjects of the patient population have an AG AC genotype and further have one or more genotypic variations in 5-HTR3A and 5-HTR3B. In some embodiments all the subjects of the patient population have an AG AC genotype and further have one or more genotypic variations in the 5-HTR3A. In some embodiments all the subjects of the patient population have an AG AC genotype and further have one or more genotypic variations in the 5-HTR3B. In some embodiments, all the subjects of the patient population have a genotype of AG, at least one genotypic variant in the 5-HTR3B (e.g., AC) and at least one genotypic variant in the non-coding and/or coding region of the serotonin transporter gene (e.g., one or more variations in the 5-HTTLPR and/or 3-untranslated region of SLC6A4 gene that encodes the serotonin transporter). In some embodiments, all the subjects of the patient population have an AG genotype and one or more further genotypic variations in the 5-HTR3A (e.g., a GG genotype), but do not have an LL genotype or a TT genotype. In some embodiments, all the subjects of the patient population have an AG genotype and at least one genotypic variation in the 5-HTR3A (e.g., a GG genotype), but do not have an LL TT genotype. In some embodiments, all the subjects of the patient population have an AG genotype and one or more genotypic variations in the 5-HTR3B but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more genotypic variations in the 5-HTR3B but do not have any of the genotypes selected from GG genotype, LL (rs4795541-LL) genotype, and TT (rs1042173-TT) genotype.

In some embodiments, all the subjects of the patient population have a genotype of AG+, wherein all the patients in the population have an AG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, all the subjects of the patient population have an AG genotype and further have a genotypic variation in the non-coding region of the SLC6A4 gene that encodes the serotonin transporter. In some embodiments, all the subjects of the patient population have an AG genotype and further have a genotypic variation in the 5-HTTLPR. In further embodiments, all the subjects of the patient population have an AG genotype and further have an LL (rs4795541-LL) genotype. In other embodiments, all the subjects of the patient population have an AG genotype and further have one or more genotypic variations in the 5-HTTLPR, but do not have an LL genotype. In some embodiments, all the subjects of the patient population have an AG genotype and further have a genotypic variation in the 3′ UTR of SLC6A4. In further embodiments, all the subjects of the patient population have an AG genotype and have a TT (rs1042173-TT) genotype. In further embodiments, all the subjects of the patient population have an AG genotype and have a TT (rs1042173-TT) genotype. In other embodiments, all the subjects of the patient population have an AG genotype and further have a genotypic variation in the 3′ UTR of SLC6A4, but does not have a TT genotype. In some embodiments, all the subjects of the patient population have an AG genotype and further have a genotypic variation that alters the splicing, transcription and/or translation of SLC6A4 nucleic acids. In some embodiments, all the subjects of the patient population have an AG genotype and further have a genotypic variation in the coding region of the SLC6A4 gene that encodes the serotonin transporter. In some embodiments, all the subjects of the patient population have an AG genotype and one or more genotypic variations in the non-coding and/or coding region of the serotonin transporter gene but do not have an LL genotype or a TT genotype. In some embodiments, all the subjects of the patient population have an AG genotype and at least one genotypic variation in the 5-HTR3A (e.g., a GG genotype), but do not have an LL TT genotype. In some embodiments, all the subjects of the patient population have an AG genotype and one or more genotypic variations in the non-coding and/or coding region of the serotonin transporter gene but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more genotypic variations in the non-coding and/or coding region of the serotonin transporter gene but do not have any of the genotypes selected from GG genotype, LL (rs4795541-LL) genotype, and TT (rs1042173-TT) genotype.

In some embodiments, all the subjects of the patient population treated according to the provided methods have a genotype of GG+, wherein all the subjects of the patient population have a genotype of GG (rs1176713-GG-SNP in HTR3A)) and at least one other genotypic variation (e.g., SNP(s) or other genotypic variations) at the HTR3 AB complex. In some embodiments, all the subjects of the patient population have a genotype of GG+, wherein all the subjects in the patient population have an GG AC, GG AG or GG AC AG genotype In some embodiments, all the subjects of the patient population have an AG genotype, at least one other genotypic variant at the HTR3 AB complex, and at least one genotypic variation in the non-coding and/or coding region of the serotonin transporter gene (e.g., one or more genotypic variations in the 5-HTTLPR and/or 3′ UTR of SLC6A4). In some embodiments, all the subjects of the patient population have an AG genotype and at least one other genotypic variant at the HTR3 AB complex, but do not have an LL (rs4795541-LL) genotype or a TT (rs1042173-TT) genotype. In some embodiments, all the subjects of the patient population have an AG genotype and at least one other genotypic variant at the HTR3 AB complex, but do not have an LL TT genotype.

In some embodiments, all the subjects of the patient population have a genotype of GG+, wherein all the subjects of the patient population have a GG (rs1176713-GG-SNP in HTR3A) genotype and one or more further genotypic variations (e.g., SNP(s) or other genotypic variations) in the 5-HTR3A receptor, one or more genotypic variations in the 5-HTR3B receptor, and/or one or more genotypic variation in the 5-HTTLPR or the 3′ UTR of the SLC6A4 gene.

In some embodiments, all the subjects of the patient population have a genotype of GG+, wherein all the subjects in the patient population have an GG (rs1176713-GG) genotype and one or more further genotypic variations in the 5-HTR3A. In some embodiments, all the subjects of the patient population have a genotype of GG+ has a GG and an AG (rs1150226-AG) genotype. In some embodiments, all the subjects of the patient population have a GG and AG genotype and further have one or more genotypic variations in the 5-HTR3A. In some embodiments, all the subjects of the patient population have a GG genotype, one or more further genotypic variations in the 5-HTR3A, and further have at least one genotypic variation in the non-coding and/or coding region of the serotonin transporter gene (e.g., one or more variations in the 5-HTTLPR and/or 3′ UTR of SLC6A4). In some embodiments, all the subjects of the patient population have a genotype of GG+, wherein all the subjects in the patient population have a GG AC LL/TT, GG AG LL/TT or GG AC AG LL/TT genotype. In some embodiments, all the subjects of the patient population have GG genotype and one or more further genotypic variations in the 5-HTR3A (e.g., an AG genotype), but do not have an LL genotype or a TT genotype. In some embodiments, all the subjects of the patient population have a GG genotype and one or more further genotypic variations in the 5-HTR3A (e.g., an AG genotype), but do not have an LL TT genotype.

In some embodiments, all the subjects of the patient population have a genotype of GG+, wherein all the subjects of the patient population have GG genotype and one or more genotypic variations in the 5-HTR3B. In some embodiments, all the subjects of the patient population have a GG genotype and an AC (rs17614942-AC) genotype. In further embodiments all the subjects of the patient population have an GG AC genotype and further have one or more genotypic variations in 5-HTR3A and 5-HTR3B. In some embodiments all the subjects of the patient population have an GG AC genotype and further have one or more genotypic variations in the 5-HTR3A. In some embodiments all the subjects of the patient population have a GG AC genotype and further have one or more genotypic variations in the 5-HTR3B. In some embodiments, all the subjects of the patient population have a genotype of GG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, all the subjects of the patient population have GG genotype and one or more further genotypic variations in the 5-HTR3A (e.g., an AG genotype), but do not have an LL genotype or a TT genotype. In some embodiments, all the subjects of the patient population have a GG genotype and at least one genotypic variation in the 5-HTR3A (e.g., an AG genotype), but do not have an LL TT genotype.

In some embodiments, all the subjects of the patient population have a genotype of GG+, wherein all the subjects of the patient population have a GG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, all the subjects of the patient population have a GG genotype and further have a genotypic variation in the non-coding region of the SLC6A4 gene that encodes the serotonin transporter. In some embodiments, all the subjects of the patient population have a GG genotype and further have a genotypic variation in the 5-HTTLPR. In further embodiments, all the subjects of the patient population have a GG genotype and further have an LL (rs4795541-LL) genotype. In other embodiments, all the subjects of the patient population have a GG genotype and further have one or more genotypic variations in the 5-HTTLPR, but do not have an LL genotype. In some embodiments, all the subjects of the patient population have a GG genotype and further have a genotypic variation in the 3′ UTR of SLC6A4. In further embodiments, all the subjects of the patient population have a GG genotype and have a TT (rs1042173-TT) genotype. In further embodiments, all the subjects of the patient population have a GG genotype and have a TT (rs1042173-TT) genotype. In other embodiments, all the subjects of the patient population have a GG genotype and further have a genotypic variation in the 3′ UTR of SLC6A4, but do not have a TT genotype. In some embodiments, all the subjects of the patient population have a GG genotype and further have a genotypic variation that alters the splicing, transcription and/or translation of SLC6A4 nucleic acids. In some embodiments, all the subjects of the patient population have a GG genotype and further have a genotypic variation in the coding region of the SLC6A4. In some embodiments, all the subjects of the patient population have a GG genotype and one or more genotypic variations in the non-coding and/or coding region of the serotonin transporter gene but does not have an LL genotype or a TT genotype. In some embodiments, all the subjects of the patient population have GG genotype and at least one genotypic variation in the 5-HTR3A (e.g., an AG genotype), but do not have an LL TT genotype.

In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a GG genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have an LL genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a TT genotype. In some embodiments all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a LL and TT genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have any of the genotypes selected from GG genotype, LL (rs4795541-LL) genotype, and TT (rs1042173-TT) genotype.

In some embodiments, all the subjects of the patient population have mild AUD at baseline. In some embodiments, all the subjects of the patient population have moderate AUD at baseline. In some embodiments, all the subjects of the patient population have severe AUD at baseline. In some embodiments, the provided methods are effective in converting severe AUD into a moderate or mild AUD for the subjects of the patient population. In some embodiments, the provided methods are effective in reducing craving for alcohol for the subjects of the patient population.

In some embodiments, the treatment period is at least six months, e.g., at least 9 months, at least 12 months, at least 24 months, such as 6-12 months, 9-12 months, 1-2 years, etc. In some embodiments, the treatment period during which the subject is administered the ondansetron is at least six months. In some embodiments the treatment period is at least 9 months, at least 12 months, or at least 24 months. In further embodiments, the treatment period is 6-12 months, 9-12 months, or 1-2 years.

In some embodiments, the dosing regimen is effective in treating the patient population to achieve a reduction of percent heavy drinking days (PHDD) in months 5 and/or 6 of the treatment period compared to baseline, wherein the reduction of PHDD in the patient population is statistically significantly higher than that of the placebo-controlled patient population or a patient population having only a single SNP selected from AC alone, AG alone, or GG alone.

In some embodiments, the dosing regimen is effective in treating the patient population to achieve 2-15, 2-10, 2-5 more days of no drinking days (NDD) in months 5 and/or 6 of the treatment period, than that of the placebo-controlled patient population. In some embodiments, the dosing regimen is effective in treating the patient population to achieve 2-15, 2-10, 2-5 or 2-4 more days of no drinking days (NDD) in months 5 and/or 6 of the treatment period, than that of the placebo-controlled patient population.

In some embodiments, the dosing regimen is effective in treating the patient population to achieve a reduction of total alcohol consumption (TAC) in months 5 and/or 6 of the treatment period, wherein the reduction of TAC in the patient population is higher than that of the placebo-controlled patient population.

In some embodiments, the dosing regimen is effective in treating the patient population to achieve a reduction of drinks per drinking day (DDD) in months 5 and/or 6 of the treatment period, wherein the reduction of DDD in the patient population is higher than that of the placebo-controlled patient population.

In some embodiments, when compared to the placebo-controlled patient population, the dosing regimen is effective in treating the patient population to achieve 1, 2, 3, or each of (a) a reduction of percent heavy drinking days in months 5 and/or 6 of the treatment period compared to baseline by at least 5%, 10% or 12% greater, such as 5-50%, 10-40%, or 15-35% greater; (b) 2-15, 2-10, 2-5 or 2-4 more days of no drinking days in month 6 of the treatment period; (c) a reduction of total alcohol consumption in month 6 of the treatment period compared to baseline by 5-40%, 5-35%, or 7-30% greater; and (d) a reduction of 1-3 more drinks per drinking day in month 6 of the treatment period compared to baseline.

In some embodiments, when compared to the placebo-controlled patient population, the dosing regimen is effective in treating the patient population to achieve (a) a reduction of percent heavy drinking days in months 5 and/or 6 of the treatment period compared to baseline by at least 5%, 10% or 12% greater, such as 5-50%, 10-40%, or 15-35% greater; (b) 2-15, 2-10, 2-5 or 2-4 more days of no drinking days in month 6 of the treatment period; (c) a reduction of total alcohol consumption in month 6 of the treatment period compared to baseline by 5-40%, 5-35%, or 7-30% greater; and (d) a reduction of 1-3 more drinks per drinking day in month 6 of the treatment period compared to baseline.

In some embodiments, when compared to the placebo-controlled patient population, the dosing regimen is effective in treating the patient population to achieve 1, 2, 3, or each of (a) a reduction of percent heavy drinking days in months 5 and/or 6 of the treatment period compared to baseline by at least 12% greater, such as 15-35% greater; (b) 2-4 more days of no drinking days in month 6 of the treatment period; (c) a reduction of total alcohol consumption in month 6 of the treatment period compared to baseline by 7-30% greater; and (d) a reduction of 1-3 more drinks per drinking day in month 6 of the treatment period compared to baseline.

In some embodiments, when compared to the placebo-controlled patient population, the dosing regimen is effective in treating the patient population to achieve ((a) a reduction of percent heavy drinking days in months 5 and/or 6 of the treatment period compared to baseline by at least 12% greater, such as 15-35% greater; (b) 2-4 more days of no drinking days in month 6 of the treatment period; (c) a reduction of total alcohol consumption in month 6 of the treatment period compared to baseline by 7-30% greater; and (d) a reduction of 1-3 more drinks per drinking day in month 6 of the treatment period compared to baseline.

In some embodiments, during the treatment period, the ondansetron is administered to the subject daily in an amount ranging from about 0.20 mg to about 1.5 mg. In some embodiments, the ondansetron is administered to the subject daily in an amount ranging from about 0.25 mg to about 1 mg. In some embodiments, the ondansetron is administered to the subject daily in an amount ranging from about 0.4 mg to about 0.8 mg. In some embodiments, the ondansetron is administered to the subject twice a day (b.i.d.), with each dose of about 0.33 mg.

In further embodiments, the provided methods further comprise determining or having determined the subject as belonging to the patient population.

On additional embodiments, the disclosure provides a method of altering the drinking habit of a subject who is a heavy drinker and (1) has a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter, and/or (2) does not have a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter; the method comprising:
administering ondansetron to the subject in an amount effective to achieve one or more (preferably, two or more, three or more, or four or more) of the following effects (a)-(e) in one or both of the last two months of a treatment period if the treatment period is five months or below, or in month 5 and/or month 6, or one or both of the last two months of the treatment period, if the treatment period is six months or longer, thereby altering the subject's drinking habit:
(a) no heavy drinking days (HHDs), wherein an HDD is a day with ethanol consumption of 60 grams or more for males and 40 grams or more for females;
(b) a reduction of percent heavy drinking days of at least 10% (for example, a reduction of at least 20%, 30%, 40%, 50%, 60%, preferably, at least 10% in addition to placebo effect) compared to that observed for the 4 weeks prior to the treatment period (“baseline”);
(c) at least two (for example, at least 3, 4, 5, 6, 7, 8, 9, 10, 11, 12, 15, 20, etc., preferably, at least two in addition to placebo effect) no drinking days;
(d) a reduction of total alcohol consumption of at least 5% (for example, a reduction of at least 10%, 15%, 20%, 30%, 40%, etc., preferably, at least 5% in addition to placebo effect) compared to baseline; and
(e) a reduction of drinks per drinking day of at least one drink (for example, a reduction of at least 2, 3, 4, 5, or 6 drinks etc., preferably, at least one drink in addition to placebo effect), wherein each drink contains about 10 grams of ethanol,
   wherein the heavy drinker has the following baseline characteristics: ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days.

In some embodiments, the subject has a genotype of AG+, wherein the subject has a genotype of AG (rs1150226-AG SNP in HTR3A) and at least one other genotypic variation (e.g., SNP(s) or other genotypic variations) at the HTR3 AB complex. In some embodiments, the subject has a genotype of AG+, wherein the subject has an AG AC, AG GG or AG AC GG genotype. In some embodiments, the subject has a genotype of AG+, wherein the subject has an AG AC genotype. In some embodiments, the subject has a genotype of AG and at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, the subject has a genotype of AG and at least one other genotypic variant at the HTR3 AB complex, but does not have an LL (rs4795541-LL) genotype or a TT (rs1042173-TT) genotype. In some embodiments, the subject has a genotype of AG and at least one other genotypic variant at the HTR3 AB complex, but does not have an LL TT genotype. In some embodiments, the subject has a genotype of AG and at least one other genotypic variant at the HTR3 AB complex, but does not have a GG genotype. In some embodiments, the subject has a genotype of AG and at least one other genotypic variant at the HTR3 AB complex, but does not have more than two, preferably, does not have more than one, more preferably, does not have any one, of the genotypes selected from GG genotype, LL (rs4795541-LL) genotype, and TT (rs1042173-TT) genotype. In some embodiments, the subject has a genotype of AG and at least one other genotypic variant at the HTR3 AB complex, but does not have any of the genotypes selected from GG genotype, LL (rs4795541-LL) genotype, and TT (rs1042173-TT) genotype. In some embodiments, the subject has a genotype of AG AC and optionally at least one other genotypic variant at the HTR3 AB complex, but does not have a GG genotype. In some embodiments, the subject treated according to the provided methods has a genotype of AG AC and optionally at least one other genotypic variant at the HTR3 AB complex, but does not have more than two, preferably, does not have more than one, more preferably, does not have any one, of the genotypes selected from GG genotype, LL (rs4795541-LL) genotype, and TT (rs1042173-TT) genotype. In some embodiments, the subject has a genotype of AG AC and optionally at least one other genotypic variant at the HTR3 AB complex, but does not have any of the genotypes selected from GG genotype, LL (rs4795541-LL) genotype, and TT (rs1042173-TT) genotype.

In some embodiments, the subject has a genotype of AG+, wherein the subject has an AG genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter.

In some embodiments, the subject having a genotype of AG+ has an AG (rs1150226-AG) genotype and one or more further genotypic variations in the 5-HTR3A. In some embodiments, the subject having a genotype of AG+ has an AG and a GG (rs1176713-GG-SNP in HTR3A) genotype. In some embodiments, the subject having a genotype of AG+ does not have a GG (rs1176713-GG-SNP in HTR3A) genotype. In some embodiments, the subject has an AG and GG genotype and further has one or more genotypic variations in the 5-HTR3A. In some embodiments, the subject has an AG genotype, one or more further genotypic variations in the 5-HTR3A, and further has at least one genotypic variation in the non-coding and/or coding region of the serotonin transporter gene (e.g., one or more variations in the 5-HTTLPR and/or 3′ UTR of SLC6A4). In some embodiments, the subject has a genotype of AG+, wherein the subject has an AG AC LL/TT, AG GG LL/TT or AG AC GG LL/TT genotype. In some embodiments, the subject has an AG genotype and one or more further genotypic variations in the 5-HTR3A (e.g., a GG genotype), but does not have an LL TT genotype. In some embodiments, the subject has an AG genotype and one or more further genotypic variations in the 5-HTR3A (e.g., a GG genotype), but does not have an LL TT genotype. In some embodiments, the subject has an AG genotype and one or more further genotypic variations in the 5-HTR3A, but does not have more than two, preferably, does not have more than one, more preferably, does not have any one of the genotypes selected from GG genotype, LL (rs4795541-LL) genotype, and TT (rs1042173-TT) genotype. In some embodiments, the subject has an AG genotype and one or more further genotypic variations in the 5-HTR3A but does not have any of the genotypes selected from GG genotype, LL (rs4795541-LL) genotype, and TT (rs1042173-TT) genotype.

In some embodiments, the subject having a genotype of AG+ has an AG genotype and one or more genotypic variations in the 5-HTR3B. In some embodiments, the subject has an AG genotype and an AC (rs17614942-AC) genotype. In further embodiments the subject has an AG AC genotype and further has one or more genotypic variations in 5-HTR3A and 5-HTR3B. In some embodiments the subject has an AG AC genotype and further has one or more genotypic variations in the 5-HTR3A. In some embodiments the subject has an AG AC genotype and further has one or more genotypic variations in the 5-HTR3B. In some embodiments, the subject has a genotype of AG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, the subject has an AG genotype and one or more further genotypic variations in the 5-HTR3A (e.g., a GG genotype), but does not have an LL genotype or a TT genotype. In some embodiments, the subject has an AG genotype and at least one genotypic variation in the 5-HTR3A (e.g., a GG genotype), but does not have an LL TT genotype. In some embodiments, the subject has an AG genotype and one or more genotypic variations in the 5-HTR3B but does not have more than two, preferably, does not have more than one, more preferably, does not have any one of the genotypes selected from GG genotype, LL (rs4795541-LL) genotype, and TT (rs1042173-TT) genotype. In some embodiments, the subject has an AG genotype and one or more genotypic variations in the 5-HTR3B but does not have any of the genotypes selected from GG genotype, LL (rs4795541-LL) genotype, and TT (rs1042173-TT) genotype.

In some embodiments, the subject having a genotype of AG+ has an AG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, the subject has an AG genotype and further has a genotypic variation in the non-coding region of the SLC6A4. In some embodiments, the subject has an AG genotype and further has a genotypic variation in the 5-HTTLPR. In further embodiments, the subject has an AG genotype and further has an LL (rs4795541-LL) genotype. In other embodiments, the subject has an AG genotype and further has one or more genotypic variations in the 5-HTTLPR but does not have an LL genotype. In some embodiments, the subject has an AG genotype and further has a genotypic variation in the 3′ UTR of SLC6A4. In further embodiments, the subject has an AG genotype and has a TT (rs1042173-TT) genotype. In further embodiments, the subject has an AG genotype and has a TT (rs1042173-TT) genotype. In other embodiments, the subject has an AG genotype and further has a genotypic variation in the 3′ UTR of SLC6A4 but does not have a TT genotype. In some embodiments, the subject has an AG genotype and further has a genotypic variation that alters the splicing, transcription and/or translation of SLC6A4 nucleic acids. In some embodiments, the subject has an AG genotype and further has a genotypic variation in the coding region of the SLC6A4 gene that encodes the serotonin transporter. In some embodiments, the subject having a genotype of AG+ has an AG genotype and one or more genotypic variations in the non-coding and/or coding region of the serotonin transporter gene but does not have an LL genotype or a TT genotype. In some embodiments, the subject has an AG genotype and at least one genotypic variation in the 5-HTR3A (e.g., a GG genotype), but does not have an LL TT genotype. In some embodiments, the subject has an AG genotype and one or more genotypic variations in the non-coding and/or coding region of the serotonin transporter gene but does not have more than two, preferably, does not have more than one, more preferably, does not have any one, of the genotypes selected from GG genotype, LL (rs4795541-LL) genotype, and TT (rs1042173-TT) genotype. In some embodiments, the subject has an AG genotype and one or more genotypic variations in the non-coding and/or coding region of the serotonin transporter gene but does not have any of the genotypes selected from GG genotype, LL (rs4795541-LL) genotype, and TT (rs1042173-TT) genotype.

In some embodiments, the subject has a genotype of GG+, wherein the subject has a genotype of GG (rs1176713-GG-SNP in HTR3A)) and at least one other genotypic variation (e.g., SNP(s) or other genotypic variations) at the HTR3 AB complex. In some embodiments, the subject has a genotype of GG+, wherein the subject has an GG AC, GG AG or GG AC AG genotype. In some embodiments, the subject has a genotype of AG and at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, the subject has a genotype of AG and at least one other genotypic variant at the HTR3 AB complex but does not have an LL (rs4795541-LL) genotype or a TT (rs1042173-TT) genotype. In some embodiments, the subject has a genotype of AG and at least one other genotypic variant at the HTR3 AB complex but does not have an LL TT genotype.

In some embodiments, the subject has a genotype of GG+, wherein the subject has an GG (rs1176713-GG-SNP in HTR3A) genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter.

In some embodiments, the subject having a genotype of GG+ has an GG (rs1176713-GG) genotype and one or more further genotypic variations in the 5-HTR3A. In some embodiments, the subject having a genotype of GG+ has a GG and an AG (rs1150226-AG) genotype. In some embodiments, the subject has a GG and AG genotype and further has one or more genotypic variations in the 5-HTR3A. In some embodiments, the subject has a GG genotype, one or more further genotypic variations in the 5-HTR3A, and further has at least one genotypic variation in the non-coding and/or coding region of the serotonin transporter gene (e.g., one or more variations in the 5-HTTLPR and/or 3′ UTR of SLC6A4). In some embodiments, the subject has a genotype of GG+, wherein the subject has an GG AC LL/TT, GG AG LL/TT or GG AC AG LL/TT genotype. In some embodiments, the subject has a GG genotype and one or more further genotypic variations in the 5-HTR3A (e.g., an AG genotype), but does not have an LL genotype or a TT genotype. In some embodiments, the subject has a GG genotype and one or more further genotypic variations in the 5-HTR3A (e.g., an AG genotype), but does not have an LL TT genotype.

In some embodiments, the subject having a genotype of GG+ has a GG genotype and one or more genotypic variations in the 5-HTR3B. In some embodiments, the subject has a GG genotype and an AC (rs17614942-AC) genotype. In further embodiments the subject has an GG AC genotype and further has one or more genotypic variations in 5-HTR3A and 5-HTR3B. In some embodiments the subject has a GG AC genotype and further has one or more genotypic variations in the 5-HTR3A. In some embodiments the subject has a GG AC genotype and further has one or more genotypic variations in the 5-HTR3B. In some embodiments, the subject has a genotype of GG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, the subject has a GG genotype and one or more further genotypic variations in the 5-HTR3A (e.g., an AG genotype), but does not have an LL genotype or a TT genotype. In some embodiments, the subject has a GG genotype and at least one genotypic variation in the 5-HTR3A (e.g., an AG genotype), but does not have an LL TT genotype.

In some embodiments, the subject having a genotype of GG+ has a GG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, the subject has a GG genotype and further has a genotypic variation in the non-coding region of the SLC6A4 gene that encodes the serotonin transporter. In some embodiments, the subject has a GG genotype and further has a genotypic variation in the 5-HTTLPR. In further embodiments, the subject has a GG genotype and further has an LL (rs4795541-LL) genotype. In other embodiments, the subject has a GG genotype and further has one or more genotypic variations in the 5-HTTLPR, but does not have an LL genotype. In some embodiments, the subject has a GG genotype and further has a genotypic variation in the 3′ UTR of SLC6A4. In further embodiments, the subject has a GG genotype and has a TT (rs1042173-TT) genotype. In further embodiments, the subject has a GG genotype and has a TT (rs1042173-TT) genotype. In other embodiments, the subject has a GG genotype and further has a genotypic variation in the 3′ UTR of SLC6A4, but does not have a TT genotype. In some embodiments, the subject has a GG genotype and further has a genotypic variation that alters the splicing, transcription and/or translation of SLC6A4 nucleic acids. In some embodiments, the subject has a GG genotype and further contains a genotypic variation in the coding region of the SLC6A4 gene that encodes the serotonin transporter. In some embodiments, the subject having a genotype of GG+ has a GG genotype and one or more genotypic variations in the non-coding and/or coding region of the serotonin transporter gene but does not have an LL genotype or a TT genotype. In some embodiments, the subject has a GG genotype and at least one genotypic variation in the 5-HTR3A (e.g., an AG genotype), but does not have an LL TT genotype.

In some embodiments, the subject has mild AUD at baseline. In some embodiments, the subject has moderate AUD at baseline. In some embodiments, the subject has severe AUD at baseline. In some embodiments, the provided methods are effective in converting the subject's severe AUD into a moderate or mild AUD. In some embodiments, the provided methods are effective in reducing the subject's craving for alcohol.

In some embodiments, the present disclosure provides a method of treating alcohol dependency of a subject who is a heavy drinker and (1) has a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter, and/or (2) does not have a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter; the method comprising:
administering to the subject ondansetron in an amount effective to achieve one or more (preferably, two or more, three or more, or four or more) of the following (a)-(e), in one or both of the last two months of a treatment period if the treatment period is five months or below, or in month 5 and/or month 6, or one or both of the last two months of the treatment period, if the treatment period is six months or longer:
(a) no heavy drinking days (HHDs), wherein an HDD is a day with ethanol consumption of 60 grams or more for males and 40 grams or more for females;
(b) a reduction of percent heavy drinking days of at least 10% (for example, a reduction of at least 20%, 30%, 40%, 50%, or 60%, preferably, at least 10% in addition to placebo effect) compared to that observed for the 4 weeks prior to the treatment period (“baseline”);
(c) at least two (for example, at least 2, 3, 4, 5, 6, 7, 8, 9, 10, 11, 12, 15, 20, etc., preferably, at least two in addition to placebo effect) no drinking days;
(d) a reduction of total alcohol consumption of at least 5% (for example, a reduction of at least 10%, 15%, 20%, 30%, 40%, etc., preferably, at least 5% in addition to placebo effect) compared to baseline; and
(e) a reduction of drinks per drinking day of at least one drink (for example, a reduction of at least 2, 3, 4, 5, or 6 drinks etc., preferably, at least one drink in addition to placebo effect), wherein each drink contains about 10 grams of ethanol,
   wherein the heavy drinker has the following baseline characteristics: ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days.

In some embodiments, the subject has mild AUD at baseline. In some embodiments, the subject has moderate AUD at baseline. In some embodiments, the subject has severe AUD at baseline. In some embodiments, the provided methods are effective in converting the subject's severe AUD into a moderate or mild AUD. In some embodiments, the provided methods are effective in reducing the subject's craving for alcohol.

In some embodiments, the disclosure provides a method of predicting the likelihood of a subject having alcohol use disorder achieving zero heavy drinking days (HDDs) in one or both of the last two months of a treatment period, if the treatment period is five months or below, or in month 5 and/or month 6, or one or both of the last two months of the treatment period, if the treatment period is six months or longer, wherein an HDD is a day with ethanol consumption of 60 grams or more for males and 40 grams or more for females, the method comprising:
(1) administering ondansetron to a patient population characterized as (i) being heavy drinkers; and (ii) having a genotype of AG+, i.e., rs1150226-AG SNP in HTR3A (“AG”) and optionally any one or more of other SNPs at the HTR3 AB complex or the serotonin transporter, or a genotype of GG+, i.e., rs1176713-GG SNP in HTR3A (“GG”) and optionally any one or more of other SNPs at the HTR3 AB complex or the serotonin transporter; and/or (iii) not having a genotype of LL or TT, wherein LL is rs4795541-LL genotype of the insertion-deletion polymorphism (5′-HTTLPR) in the 5′-regulatory region and TT is rs1042173-TT SNP in the 3′-untranslated region of SLC6A4 gene that encodes the serotonin transporter;
(2) identifying the patient population as responsive to ondansetron treatment if the ondansetron can be administered with a dosing regimen that is effective in treating the patient population such that the percentage of the patient population having zero HDD (PNHDD) in one or both of the last two months of the treatment period, if the treatment period is five months or below, or in month 5 and/or month 6, or one or both of the last two months of the treatment period, if the treatment period is six months or longer, is higher than that of a placebo-controlled patient population, for example, at least 10%, 20%, 30%, 40%, or 50% higher than that of the placebo-controlled patient population, e.g., 25-50%, 30-40%, or 30-35% higher than that of the placebo-controlled patient population;
wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days; and
(3) determining or having determined whether the subject belongs to the patient population by comparing the genotype and drinking pattern with those of the patient population, wherein a subject belonging to the patient population has a higher chance of achieving zero HDD.

In some embodiments, the disclosure further provides a method of treating a subject determined to be in need of treatment as having a higher chance of achieving zero HDD according to the above method, comprising administering the subject ondansetron with a dosing regimen that is effective in treating the patient population such that the percentage of the patient population having zero HDD (PNHDD) in one or both of the last two months of the treatment period, if the treatment period is five months or below, or in month 5 and/or month 6, or one or both of the last two months of the treatment period, if the treatment period is six months or longer, is higher than that of a placebo-controlled patient population, for example, at least 10%, 20%, 30%, 40%, or 50% higher than that of the placebo-controlled patient population, e.g., 25-50%, 30-40%, or 30-35% higher than that of the placebo-controlled patient population. In some embodiments, during the treatment period, the ondansetron is administered to the subject daily in an amount ranging from about 0.20 mg to about 1.5 mg. In some embodiments, the subject determined to be likely to achieve zero heavy drinking days (HDDs) according to the above method is administered to the subject daily in an amount ranging from about 0.25 mg to about 1 mg. In some embodiments, the ondansetron is administered to the subject daily in an amount ranging from about 0.4 mg to about 0.8 mg. In some embodiments, the ondansetron is administered to the subject twice a day (b.i.d.), with each dose of about 0.33 mg.

In some embodiments, all the subjects of the patient population have a genotype of AG+, wherein all the subjects of the patient population have a genotype of AG (rs1150226-AG SNP in HTR3A) and at least one other genotypic variation (e.g., SNP(s) or other genotypic variations) at the HTR3 AB complex. In some embodiments, all the subjects of the patient population have a genotype of AG+, wherein all the subjects in the patient population have an AG AC, AG GG or AG AC GG genotype. In some embodiments, all the subjects of the patient population have a genotype of AG+, wherein all the subjects of the patient population have an AG AC genotype. In some embodiments, all the subjects of the patient population have a genotype of AG, at least one other genotypic variant at the HTR3 AB complex and at least one genotypic variation in the non-coding and/or coding region of the serotonin transporter gene (e.g., one or more genotypic variations in the 5-HTTLPR and/or 3′ UTR of SLC6A4). In some embodiments, all the subjects of the patient population have a genotype of AG and at least one other genotypic variant at the HTR3 AB complex, but do not have an LL (rs4795541-LL) genotype or a TT (rs1042173-TT) genotype. In some embodiments, all the subjects of the patient population have a genotype of AG and at least one other genotypic variant at the HTR3 AB complex, but do not have an LL TT genotype. In some embodiments, all the subjects of the patient population have a genotype of AG+, wherein all the subjects of the patient population have an AG genotype and one or more further genotypic variations (e.g., SNP(s) or other genotypic variations) in the 5-HTR3A receptor, one or more genotypic variations in the 5-HTR3B receptor, and/or one or more genotypic variations in the 5-HTTLPR or the 3′ UTR of the SLC6A4 gene that encodes the serotonin transporter. In some embodiments, all the subjects of the patient population have a genotype of AG and at least one other genotypic variant at the HTR3 AB complex, but do not have a GG genotype. In some embodiments, all the subjects of the patient population have a genotype of AG and at least one other genotypic variant at the HTR3 AB complex, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have a genotype of AG and at least one other genotypic variant at the HTR3 AB complex, but do not have any of the genotypes selected from GG genotype, LL (rs4795541-LL) genotype, and TT (rs1042173-TT) genotype. In some embodiments, all the subjects of the patient population have a genotype of AG AC and optionally at least one other genotypic variant at the HTR3 AB complex, but do not have a GG genotype. In some embodiments, all the subjects of the patient population have a genotype of AG AC and optionally at least one other genotypic variant at the HTR3 AB complex, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have a genotype of AG AC and optionally at least one other genotypic variant at the HTR3 AB complex, but do not have any of the genotypes selected from GG genotype, LL (rs4795541-LL) genotype, and TT (rs1042173-TT) genotype.

In some embodiments, all the subjects of the patient population have an AG+ genotype wherein all the subjects of the patient population have an AG (rs1150226-AG) genotype and one or more further genotypic variations in the 5-HTR3A. In some embodiments, all the subjects of the patient population have an AG genotype and a GG (rs1176713-GG-SNP in HTR3A) genotype. In some embodiments, all the subjects of the patient population have an AG genotype but do not have a GG (rs1176713-GG-SNP in HTR3A) genotype. In some embodiments, all the subjects of the patient population have an AG GG genotype and further have one or more genotypic variations in the 5-HTR3A. In some embodiments, all the subjects of the patient population have an AG genotype, one or more further genotypic variations in the 5-HTR3A, and further have at least one genotypic variation in the non-coding and/or coding region of the serotonin transporter gene (e.g., one or more variations in the 5-HTTLPR and/or 3′ UTR of SLC6A4). In some embodiments, all the subjects of the patient population have a genotype of AG+, wherein all the subjects in the patient population have an AG AC, AG GG or AG AC GG genotype. In some embodiments, all the subjects of the patient population have an AG genotype and one or more further genotypic variations in the 5-HTR3A (e.g., a GG genotype), but do not have an LL genotype or a TT genotype. In some embodiments, all the subjects of the patient population have AG genotype and one or more further genotypic variations in the 5-HTR3A (e.g., a GG genotype), but do not have a LL TT genotype. In some embodiments, all the subjects of the patient population have an AG genotype and one or more further genotypic variations in the 5-HTR3A, but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more further genotypic variations in the 5-HTR3A, but do not have any of the genotypes selected from GG genotype, LL (rs4795541-LL) genotype, and TT (rs1042173-TT) genotype.

In some embodiments, all the subjects of the patient population have a genotype of AG+ wherein all the subjects of the patient population have AG genotype and one or more genotypic variations in the 5-HTR3B. In some embodiments, all the subjects of the patient population have an AG genotype and an AC (rs17614942-AC) genotype. In further embodiments all the subjects of the patient population have an AG AC genotype and further have one or more genotypic variations in 5-HTR3A and 5-HTR3B. In some embodiments all the subjects of the patient population have an AG AC genotype and further have one or more genotypic variations in the 5-HTR3A. In some embodiments all the subjects of the patient population have an AG AC genotype and further have one or more genotypic variations in the 5-HTR3B. In some embodiments, all the subjects of the patient population have a genotype of AG, at least one genotypic variant in the 5-HTR3B (e.g., AC) and at least one genotypic variant in the non-coding and/or coding region of the serotonin transporter gene (e.g., one or more variations in the 5-HTTLPR and/or 3-untranslated region of SLC6A4 gene that encodes the serotonin transporter). In some embodiments, all the subjects of the patient population have an AG genotype and one or more further genotypic variations in the 5-HTR3A (e.g., a GG genotype), but do not have an LL genotype or a TT genotype. In some embodiments, all the subjects of the patient population have an AG genotype and at least one genotypic variation in the 5-HTR3A (e.g., a GG genotype), but do not have an LL TT genotype. In some embodiments, all the subjects of the patient population have an AG genotype and one or more genotypic variations in the 5-HTR3B but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more genotypic variations in the 5-HTR3B but do not have any of the genotypes selected from GG genotype, LL (rs4795541-LL) genotype, and TT (rs1042173-TT) genotype.

In some embodiments, all the subjects of the patient population have a genotype of AG+, wherein all the patients in the population have an AG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, all the subjects of the patient population have an AG genotype and further have a genotypic variation in the non-coding region of the SLC6A4 gene that encodes the serotonin transporter. In some embodiments, all the subjects of the patient population have an AG genotype and further have a genotypic variation in the 5-HTTLPR. In further embodiments, all the subjects of the patient population have an AG genotype and further have an LL (rs4795541-LL) genotype. In other embodiments, all the subjects of the patient population have an AG genotype and further have one or more genotypic variations in the 5-HTTLPR, but do not have an LL genotype. In some embodiments, all the subjects of the patient population have an AG genotype and further have a genotypic variation in the 3′ UTR of SLC6A4. In further embodiments, all the subjects of the patient population have an AG genotype and have a TT (rs1042173-TT) genotype. In further embodiments, all the subjects of the patient population have an AG genotype and have a TT (rs1042173-TT) genotype. In other embodiments, all the subjects of the patient population have an AG genotype and further have a genotypic variation in the 3′ UTR of SLC6A4, but does not have a TT genotype. In some embodiments, all the subjects of the patient population have an AG genotype and further have a genotypic variation that alters the splicing, transcription and/or translation of SLC6A4 nucleic acids. In some embodiments, all the subjects of the patient population have an AG genotype and further have a genotypic variation in the coding region of the SLC6A4 gene that encodes the serotonin transporter. In some embodiments, all the subjects of the patient population have an AG genotype and one or more genotypic variations in the non-coding and/or coding region of the serotonin transporter gene but do not have an LL genotype or a TT genotype. In some embodiments, all the subjects of the patient population have an AG genotype and at least one genotypic variation in the 5-HTR3A (e.g., a GG genotype), but do not have an LL TT genotype. In some embodiments, all the subjects of the patient population have an AG genotype and one or more genotypic variations in the non-coding and/or coding region of the serotonin transporter gene but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have an AG genotype and one or more genotypic variations in the non-coding and/or coding region of the serotonin transporter gene but do not have any of the genotypes selected from GG genotype, LL (rs4795541-LL) genotype, and TT (rs1042173-TT) genotype.

In some embodiments, all the subjects of the patient population treated according to the provided methods have a genotype of GG+, wherein all the subjects of the patient population have a genotype of GG (rs1176713-GG-SNP in HTR3A)) and at least one other genotypic variation (e.g., SNP(s) or other genotypic variations) at the HTR3 AB complex. In some embodiments, all the subjects of the patient population have a genotype of GG+, wherein all the subjects in the patient population have an GG AC, GG AG or GG AC AG genotype. In some embodiments, all the subjects of the patient population have an AG genotype, at least one other genotypic variant at the HTR3 AB complex, and at least one genotypic variation in the non-coding and/or coding region of the serotonin transporter gene (e.g., one or more genotypic variations in the 5-HTTLPR and/or 3′ UTR of SLC6A4). In some embodiments, all the subjects of the patient population have an AG genotype and at least one other genotypic variant at the HTR3 AB complex, but do not have an LL (rs4795541-LL) genotype or a TT (rs1042173-TT) genotype. In some embodiments, all the subjects of the patient population have an AG genotype and at least one other genotypic variant at the HTR3 AB complex, but do not have an LL TT genotype.

In some embodiments, all the subjects of the patient population have a genotype of GG+, wherein all the subjects of the patient population have a GG (rs1176713-GG-SNP in HTR3A) genotype and one or more further genotypic variations (e.g., SNP(s) or other genotypic variations) in the 5-HTR3A receptor, one or more genotypic variations in the 5-HTR3B receptor, and/or one or more genotypic variation in the 5-HTTLPR or the 3′ UTR of the SLC6A4 gene.

In some embodiments, all the subjects of the patient population have a genotype of GG+, wherein all the subjects in the patient population have an GG (rs1176713-GG) genotype and one or more further genotypic variations in the 5-HTR3A. In some embodiments, all the subjects of the patient population have a genotype of GG+ has an GG and an AG (rs1150226-AG) genotype. In some embodiments, all the subjects of the patient population have a GG and AG genotype and further have one or more genotypic variations in the 5-HTR3A. In some embodiments, all the subjects of the patient population have a GG genotype, one or more further genotypic variations in the 5-HTR3A, and further have at least one genotypic variation in the non-coding and/or coding region of the serotonin transporter gene (e.g., one or more variations in the 5-HTTLPR and/or 3′ UTR of SLC6A4). In some embodiments, all the subjects of the patient population have a genotype of GG+, wherein all the subjects in the patient population have an GG AC LL/TT, GG AG LL/TT or GG AC AG LL/TT genotype. In some embodiments, all the subjects of the patient population have GG genotype and one or more further genotypic variations in the 5-HTR3A (e.g., an AG genotype), but do not have an LL genotype or a TT genotype. In some embodiments, all the subjects of the patient population have a GG genotype and one or more further genotypic variations in the 5-HTR3A (e.g., an AG genotype), but do not have an LL TT genotype.

In some embodiments, all the subjects of the patient population have a genotype of GG+ wherein all the subjects of the patient population have GG genotype and one or more genotypic variations in the 5-HTR3B. In some embodiments, all the subjects of the patient population have a GG genotype and an AC (rs17614942-AC) genotype. In further embodiments all the subjects of the patient population have a GG AC genotype and further have one or more genotypic variations in 5-HTR3A and 5-HTR3B. In some embodiments all the subjects of the patient population have a GG AC genotype and further have one or more genotypic variations in the 5-HTR3A. In some embodiments all the subjects of the patient population have a GG AC genotype and further have one or more genotypic variations in the 5-HTR3B. In some embodiments, all the subjects of the patient population have a genotype of GG, at least one genotypic variant in the 5-HTR3B (e.g., AC) and at least one genotypic variant in the non-coding and/or coding region of the serotonin transporter gene (e.g., one or more variations in the 5-HTTLPR and/or 3′-untranslated region of SLC6A4 gene that encodes the serotonin transporter). In some embodiments, all the subjects of the patient population have GG genotype and one or more further genotypic variations in the 5-HTR3A (e.g., an AG genotype), but do not have an LL genotype or a TT genotype. In some embodiments, all the subjects of the patient population have a GG genotype and at least one genotypic variation in the 5-HTR3A (e.g., an AG genotype), but do not have an LL TT genotype.

In some embodiments, all the subjects of the patient population have a genotype of GG+, wherein all the subjects of the patient population have a GG genotype and one or more genotypic variations in the non-coding and/or coding region of the serotonin transporter gene (e.g., one or more genotypic variations in the 5-HTTLPR and/or 3′ UTR of the SLC6A4 gene that encodes the serotonin transporter). In some embodiments, all the subjects of the patient population have a GG genotype and further have a genotypic variation in the non-coding region of the SLC6A4 gene that encodes the serotonin transporter. In some embodiments, all the subjects of the patient population have a GG genotype and further have a genotypic variation in the 5-HTTLPR. In further embodiments, all the subjects of the patient population have a GG genotype and further have an LL (rs4795541-LL) genotype. In other embodiments, all the subjects of the patient population have a GG genotype and further have one or more genotypic variations in the 5-HTTLPR, but do not have an LL genotype. In some embodiments, all the subjects of the patient population have a GG genotype and further have a genotypic variation in the 3′ UTR of SLC6A4. In further embodiments, all the subjects of the patient population have a GG genotype and have a TT (rs1042173-TT) genotype. In further embodiments, all the subjects of the patient population have a GG genotype and have a TT (rs1042173-TT) genotype. In other embodiments, all the subjects of the patient population have a GG genotype and further have a genotypic variation in the 3′ UTR of SLC6A4, but do not have a TT genotype. In some embodiments, all the subjects of the patient population have a GG genotype and further have a genotypic variation that alters the splicing, transcription and/or translation of SLC6A4 nucleic acids. In some embodiments, all the subjects of the patient population have a GG genotype and further have a genotypic variation in the coding region of the SLC6A4. In some embodiments, all the subjects of the patient population have a GG genotype and one or more genotypic variations in the non-coding and/or coding region of the serotonin transporter gene but does not have an LL genotype or a TT genotype. In some embodiments, all the subjects of the patient population have GG genotype and at least one genotypic variation in the 5-HTR3A (e.g., an AG genotype), but do not have an LL TT genotype.

In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have more than two, preferably, do not have more than one, more preferably, do not have any one, of the genotypes selected from GG genotype, LL (rs4795541-LL) genotype, and TT (rs1042173-TT) genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a GG genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have an LL genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a TT genotype. In some embodiments all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have a LL and TT genotype. In some embodiments, all the subjects of the patient population have at least one genotypic variation (e.g., SNP(s) or other genotypic variations) at the HTR3 AB complex and/or at least one genotypic variant in the non-coding and/or coding region of the serotonin transporter gene (e.g., one or more variations in the 5-HTTLPR and/or 3′-untranslated region of SLC6A4 gene that encodes the serotonin transporter), but do not have any of the genotypes selected from GG genotype, LL (rs4795541-LL) genotype, and TT (rs1042173-TT) genotype.

In some embodiments, all the subjects of the patient population have mild AUD at baseline. In some embodiments, all the subjects of the patient population have moderate AUD at baseline. In some embodiments, all the subjects of the patient population have severe AUD at baseline. In some embodiments, the provided methods are effective in converting severe AUD into a moderate or mild AUD for the subjects of the patient population. In some embodiments, the provided methods are effective in reducing craving for alcohol for the subjects of the patient population.

The disclosure of U.S. Provisional Application No. 63/668,126, filed Jul. 5, 2024, is herein incorporated by reference in its entirety.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### EXAMPLES

### Example 1. Clinical Trial on Treating Alcohol Use Disorder with Ondansetron (AD04)

This example shows the design and results of a phase III, 6-month, 25-site, randomized, placebo-controlled clinical trial using ondansetron (AD04) to treat DSM-5-categorized AUD individuals who were pre-stratified into the endophenotypes of heavy or very heavy drinking individuals and possessed a pre-defined profile of genetic variants related to the serotonin transporter and serotonin-3AB receptor. Participants (N=303) presented moderate to severe AUD, >80% were men, mostly in their fifties, and ≥95% were of European descent. Low-dose AD04 (approximately 0.33 mg twice daily (b.i.d.)) or a matching placebo was administered twice daily for 6 months. Brief Behavioral Compliance Enhancement Treatment (BBCET) (Johnson B., et al. Handbook of clinical alcoholism treatment Baltimore Lippincott Williams & Wilkins. 2003a; In: Johnson, B. A., Ruiz, P., and Galanter, M. (Editors):282-301) was administered every two weeks to enhance medication compliance and clinic attendance.

### Study Design

A Phase III, 6-month, RDBPCT of ondansetron (AD04) vs. matching placebo in individuals with alcohol use disorder (AUD), all of whom had a pre-specified bio-genetic endophenotype (i.e., specific variants at the 5-HTTLR and 5-HT3 A/B receptor complex) and were pre-stratified by bio-psychosocial endophenotypes (i.e., heavy vs very heavy drinking individuals) was conducted. The trial spanned 25 sites across seven European countries, with ethics approval and adherence to relevant regulations. This study is registered at www.clinicaltrials.gov (NCT04101227).

Bio-psychosocial endophenotypes: heavy drinking individuals who consumed <10 standard drinks per drinking day (DDD) and very heavy drinking individuals who consumed ≥10 standard DDD.

Enrollees (participants, subjects) diagnosed with AUD met specific inclusion criteria, including age ≥18, heavy alcohol consumption, willingness for DNA analysis, and specific genotypes. Although abstinence was not an outcome, participants expressed a desire to reduce alcohol consumption. Compliance with treatment attendance and medication taking was optimized through participation in standardized, manual-driven, Brief Behavioral Compliance Enhancement Treatment (BBCET), and personnel were trained to use Alcohol Timeline Follow Back (TLFB) and BBCET rating scales. BBCET was supervised with recorded sessions, about 10% of which were examined for fidelity and adherence to the protocol (see supplemental materials for the list of inclusion and exclusion criteria). Participants were pre-stratified based on gender and alcohol consumption and then randomized using a permuted block design and a 1:1 allocation ratio. Medication was delivered in blinded boxes, ensuring quadruple masking. A total of 303 participants were included in the study (N=303). Full inclusion and exclusion criteria are described below.

Inclusion criteria were as follow: aged ≥18 years; reported current heavy alcohol consumption (≥60 g alcohol/day and ≥40 g alcohol/day for men and women, respectively) on six or more days in the four-week period preceding the Screening visit and were currently drinking above the WHO International guide for monitoring alcohol consumption and related harm (Johnson B, Addolorato G, Lesch O, Liu L, Rodd Z A. A critical scientific evaluation of a purportedly negative data report—response to Seneviratne et al. 2022. Front Psychiatry. 2023 Oct. 4; 14:1271229. doi: 10.3389/fpsyt.2023.1271229, PMID 37860166, PMCID PMC10582924)—defined as drinking an average of >40 g of ethanol/day for males and >20 g of ethanol/day for females for the 4 weeks prior to the screening visit; willing to provide a blood sample for DNA analysis, and had selected genotypes: LL genotype of the insertion-deletion polymorphism in the 5′ regulatory region plus rs1042173-TT SNP in the 3′-untranslated region of the SLC6A4 gene that encodes the serotonin transporter (LL/TT); and/or one, two, or three of the single nucleotide polymorphisms (SNPs) on the genes for the 5-HT3 receptor subunits: rs1150226-AG or rs1176713-GG in the gene that encodes the 5-HT3A receptor subunit, and rs17614942-AC in the gene that encodes the 5-HT3B receptor subunit. Finally, subjects had to have a negative screen for narcotics, amphetamines, and sedative-hypnotics at enrollment and had signed a written informed consent form to participate.

Exclusion criteria were as follow: a current substance use disorder diagnosis other than alcohol or nicotine use disorder; alcohol withdrawal symptoms necessitating inpatient detoxification; clinically significant somatic abnormalities (i.e., on physical examination), electrocardiography including a QTcF interval >450 ms, malignant or hematologic disease, liver enzymes ≥3 times the upper limit of normal (except for gamma glutamyl transferase), elevated bilirubin, pregnancy, lactation, taking medications with a potential effect on alcohol consumption, and the receipt of treatment for their AUD within 28 days prior to enrollment.

Details of the inclusion and exclusion criteria for this trial are shown below: Inclusion Criteria
1. The subject has signed the Informed Consent Form.
2. The subject has breath alcohol concentration (BAC) of 0.00% at the Screening and <0.02% at the Baseline visit.
3. The subject has moderate to severe diagnosis of AUD as measured by Diagnostic and Statistical Manual of Mental Disorders, 5^{th} Edition (DSM-5) criteria.
4. Males and females aged 18 and over.
5. Able to provide Timeline Follow-back Method (TLFB) alcohol consumption information for the 28-day period prior to Screening Visit.
6. A subject is eligible for participation in the study if he/she had:
   ≥6 HDDs (a heavy drinking day (HDD) is defined as a day with alcohol consumption of 60 g or more for males and 40 g or more for females) in the 4 weeks prior to the Baseline (Screening) Visit),
   an average alcohol consumption at the medium risk level (defined by the World Health Organization (WHO) “International guide for monitoring alcohol consumption and related harm” as >40 grams of ethanol/day for males and >20 grams of ethanol/day for females) for the 4 weeks prior to the Screening Visit,
   ≤14 consecutive abstinent days in the 4 weeks preceding the Screening Visit.
7. Willingness to provide a blood sample for DNA analysis at the Screening visit. The blood sample collected for DNA testing contains at least one of the following genotypes as measured by Adial's validated method:
   rs4795541-LL genotype of the insertion-deletion polymorphism (5′-HTTLPR) in the 5′-regulatory region and rs1042173-TT SNP in the 3′-untranslated region of SLC6A4 gene that encodes the serotonin transporter (LL/TT)
   rs1150226-AG SNP in HTR3A, the gene that encodes subtype A of the serotonin-3 receptor
   rs1176713-GG SNP in HTR3A, the gene that encodes subtype A of the serotonin-3 receptor
   rs17614942-AC in HTR3B, the gene that encodes subtype B of the serotonin-3 receptor
8. Expressed a wish to reduce or stop alcohol consumption.
9. Willingness to participate in behavioral and medicinal treatments for AUD.
10. Has had a stable residence in the 28 days prior to the Baseline Visit in the study and has no plans to move in the next 9 months. A stable residence is a domicile in which an individual can operate as if it were his or her own homestead and does not include shelters or halfway houses.
11. Provides contact information for 2 individuals who can be used to contact the subject.
12. Able to read and understand and complete the rating scales and questionnaires accurately, follow instructions, and make use of the behavioral treatments.
13. The subject, if female must:
   have a negative urine pregnancy test prior to the initiation of treatment and agree not to try to become pregnant during the study
   use two adequate methods of contraception (intrauterine device, oral contraceptives, progesterone implanted rods, or regular medroxyprogesterone acetate injections in addition to condom or diaphragm, or double barrier method (condom or diaphragm+spermicide)), or
   be post-menopausal having had the last natural menstruation at least 24 months prior to the Screening Visit, or
   have had a hysterectomy or been surgically sterilized prior to baseline.

### Exclusion Criteria

1. Patients with withdrawal symptoms requiring additional medication for withdrawal. If present at Screening/Baseline Visit, subjects must complete a medically supervised detoxification program prior to being able to enroll in the study.
2. Subjects with diagnosis of any of the following concomitant psychiatric disorders: non-treated, unstable schizophrenia, bipolar disorder, other psychotic disorder during the lifetime of the subject. Recent (within last 12 months) diagnosis of a major depressive disorder, post-traumatic stress disorder, panic disorder or eating disorders. Subjects with nicotine use disorder, phobic or other anxiety disorders (other than post-traumatic stress disorder or panic disorder) can be included.
3. The subject reports current or recent (within 8 weeks prior to Baseline Visit) treatment with antipsychotics or antidepressants medications, which can have an effect on serotonin receptor or transporter actions.
4. The subject has been treated with any investigational medicinal product within 30 days or 5 half-lives (whichever is longer) prior to the Baseline Visit.
5. The subject is currently participating or has recently (4 weeks prior to the Baseline Visit) participated in a treatment program for alcohol use disorders.
6. Any subject who has suicidal thoughts as evaluated by the Columbia Suicide Severity Rating Scale (C-SSRS)(i.e., has any suicidal ideation of type 4 or 5 on the C-SSRS in the last month).
7. The subject has a clinically significant untreated and unstable illness, for example, hepatic or renal insufficiency, or a cardiovascular, pulmonary, gastrointestinal, endocrine, neurological, infectious, neoplastic, or metabolic disturbance.
8. The subject has clinically significant abnormal vital signs.
9. The subject has a clinically abnormal ECG at the Screening/Baseline Visit, clinically significant cardiovascular disease requiring regular or intensive clinical monitoring, a current history of arrhythmias, or a current or past history of clinically significant QT prolongation, including:
   QTcF>450 ms (one ECG at screening and average of 3 12-lead measurements at baseline)
   serum potassium, magnesium or calcium levels outside the central laboratory's reference range
   receiving medications (within the last 7 days prior to the Baseline Visit) that have the potential of prolonging the QT interval or may require such medications during the course of the study
   clinically unstable cardiac disease, including unstable atrial fibrillation, symptomatic bradycardia, unstable congestive heart failure, active myocardial ischemia or indwelling pacemaker
   complete left bundle branch block
   history of Long QT Syndrome or an immediate family member with this condition
10. The subject with elevated liver function tests or diagnosis of hepatic failure, esophageal variceal disease or any other clinically significant hepatic disease. The clinical evidence may include any of the following: prolonged prothrombin time (International Normalized Ratio, INR≥1.7) with bilirubin >10% above the upper limit, and/or serum glutamic oxaloacetic transaminase (SGOT), and/or serum glutamic pyruvic transaminase (SGPT) and/or lactate dehydrogenase (LDH)>3× the upper limit of normal at screening.
11. The subject reports treatment, either current or within 28 days prior to the Baseline Visit, with any medications having a potential effect on alcohol consumption and related behaviors or mood. These includeopiate antagonists (e.g., naltrexone, Vivitrol®, Selincro®), glutamate antagonists (e.g., acamprosate), anticonvulsants (e.g., topiramate), serotonin reuptake inhibitors (e.g., fluoxetine), serotonin antagonists (e.g., buspirone), other antidepressants (e.g., tricyclic antidepressants or monoamine oxidase inhibitors), dopamine antagonists (e.g., haloperidol), and disulfiram (Antabuse®). Note benzodiazepines are allowed if used chronically.
12. Previous or current abuse of benzodiazepines.
13. At Baseline Visit, the subject's urine contains prescription and non-prescription drugs with abuse potential or other psychotropic agents not otherwise specified, including herbal agents such as St. John's Wort that could interfere with the drug treatment.
14. The subject has a history of allergic reactions or other known intolerance to ondansetron or other 5-HT3 antagonists.
15. Female subjects of childbearing potential who have a positive pregnancy test at Screening/Baseline Visit or are pregnant, breast feeding and who are unwilling to adhere to an acceptable form of contraception or meet the other criteria for inclusion as specified for females in the inclusion criteria (See Inclusion Criteria, Item #13).
16. The subject received in-patient or out-patient treatment for alcohol use disorder within the 28 days prior to the Baseline Visit.
17. As of the Baseline Visit, the subject is compelled to participate in an alcohol treatment program to maintain his/her liberty.
18. As of Baseline Visit, the subject is sharing a household with a subject randomized to any investigational trial of ondansetron.
19. Any other condition or therapy that in the investigator's opinion may pose a risk to the subject, prevent the subject from completing the required study procedures or interfere with the study objectives.

Enrollment occurred between February 2020 and August 2021 and included comprehensive assessments at screening and baseline (Visit 2). Participants received double-blind treatment for 6 months, with safety assessments at scheduled intervals. BBCET, administered twice monthly, aimed to enhance medication compliance and clinic attendance. Training, certification, and performance monitoring ensured consistent administration. Safety assessments occurred at 2-month intervals, including electrocardiogram (ECG) recordings. At months 3 and 6, physical examinations and other tests were conducted. The study concluded with a safety follow-up at month 7. Both active medication (ondansetron, AD04) and matching placebo were identical, supplied by Catalent, with pill-taking frequency calculated from the amount returned subtracted from the dispensed amounts. In summary, this precision medicine clinical trial used rigorous randomization, blinding, and compliance measures. Safety assessments and follow-ups were done throughout the study. Safety assessments occurred at 2-month intervals, including ECG recordings. Adverse events were collected at every study visit and as reported.

### Details of Schedule of Assessments

At screening (Visit 1), after providing written informed consent, subjects were assessed on: (1) physical health status, including medical history and vital signs, 12-lead ECG, and a comprehensive set of hematologic and biochemical studies, including a urine pregnancy test; (2) breath alcohol concentration (BAC); (3) urine drug and biochemical screens; (4) DSM-5 (American Psychiatric Association. Desk Reference to the Diagnostic Criteria from DSM-5 (2013) diagnosed AUD status (mild, moderate, or severe) and the number of AUD criteria as well as psychiatric diagnosis; (5) self-reported drinking over the past 28 days based on the timeline follow-back (TLFB)_(Sobell, L. C. & Sobell, M. B. Timeline follow-back: A technique for assessing self-reported alcohol consumption. In Measuring alcohol consumption: Psychosocial and biochemical methods. 41-72 (Humana Press, Totowa, NJ, US, 1992)); (6) suicidality based on the C-SSRS (Posner, K., et al. The American Journal of Psychiatry 168, 1266-1277 (2011));_(7) AE profile; (8) medication history; and (9) DNA analysis to identify the target genotypes i.e., rs4795541 (InDel) encoding the LL/rs1042173 and TT genotypes and/or one, two, or three of the SNPs on the genes for the 5-HT3 receptor subunits: rs1150226-AG or rs1176713-GG in the gene that encodes the 5-HT3A receptor subunit, and rs17614942-AC in the gene that encodes the 5-HT3B receptor subunit.

Eligible subjects were invited back for Visit 2 (baseline) approximately 28 days after Visit 1. Participants were re-assessed against the inclusion and exclusion criteria, and those evaluations also included physical status through the conduct of a physical examination and vital signs, BAC<0.02%, medication history since Visit 1, AEs, urine pregnancy test, drug screen, TLFB, quality of life using the WHO Quality of Life BREF (WHO. The World Health Organization Quality of Life (WHOQOL). (World Health Organization, Geneva, 1998)), general well-being using the 9-item Patient Health Questionnaire (PHQ-9 (Kroenke, K., Spitzer, R. L. & Williams, J. B. Journal of general internal medicine 16, 606-613 (2001)); nd non-pathological 12-lead ECG without a prolonged QTc (<450 ms), interval, which had been previously associated with the effects of intravenous but not oral ondansetron (Gupta, S. D., et al. Journal of natural science, biology, and medicine 2, 119-124 (2011); and Freedman, S. B., et al. Annals of emergency medicine 64, 19-25 e16 (2014)).

Participants were then randomly assigned to receive double-blind treatment (i.e., ondansetron 0.33 mg (AD04) or placebo, morning and evening), and attended their first session of a structured brief psychosocial intervention to enhance compliance with the study and pill-taking (BBCET).

After Visit 2, subjects received 24 weeks of double-blind treatment (Weeks 1 to 24). At bi-weekly intervals, subjects were administered BBCET and the clinical team collected the TLFB, AEs, pill-taking frequency, concomitant medications, and AE profile. At monthly intervals between Weeks 1 and 24, subjects were assessed on vital signs, BAC, urine pregnancy test for women, PHQ-9, CSSRS, and pill-taking frequency. At eight-week intervals during the same treatment period, subjects were assessed for safety via ECG recordings. At Weeks 12 and 24, subjects were assessed for safety by physical examination, examination of hematologic and biochemical tests, urine drug screen; and the severity of AUD illness via the DSM-5 criteria, and quality of life via the WHO Quality of Life BREF. Finally, at Week 24, to establish safety at discharge, a physical examination was done. A 4-week safety follow-up period occurred after discontinuation of treatment at Week 24, and the study concluded at Week 28.

Optimapharm managed data quality supervision, cleaning, and dataset delivery. PharPoint Research Inc. handled safety and efficacy determination.

Out of enrolled subjects, 303 received at least one dose, forming the safety population (AD04: 156; placebo: 147). Intent-to-treat analysis was employed for efficacy evaluation based on randomized treatment. An independent Data Monitoring Committee (DMC) ensured safety, reviewing unblinded data every three months. The DMC monitored adherence to study procedures and recommended study continuation without modification.

Demographic and baseline characteristics were described using summary statistics. Primary and secondary outcomes, except the modified WHO Quality BREF scale (DRL), were analyzed using a Mixed Model for Repeated Measures (MMRM) with an unstructured covariance structure for repeated measures. Restricted maximum likelihood estimation and the Kenward-Roger approximation for degrees of freedom were employed. For the drinking data, covariates in the model included the stratification factors of gender and baseline DDD category (i.e., heavy drinking individuals vs very heavy drinking individuals), together with baseline PHDD, study month (as a categorical variable), and a study month by treatment interaction. For the DRL, logistic models were used to describe the proportion of subjects with a significant categorical shift from baseline compared with study Month 6. Missing data were addressed using the MMRM analysis, assuming random missingness.

Incomplete dates were systematically imputed, minimizing bias. Reasonable imputation methods were applied for analyses requiring complete dates. Planned subgroup analyses were conducted to examine treatment effects.

The committee provided ethics approval for protecting human subjects at each participating site. The trial complied with the protocol, the International Conference of the Harmonization of Good Clinical Practice, the applicable regulatory requirement(s), and the Declaration of Helsinki.

The primary outcome measures are the change from baseline in the percentage of monthly heavy drinking days (PHDD), time frame is weeks 16-24.

Secondary outcome of this study include the following: Change from baseline in PHDD at each study month; AUD symptoms and clinical status (<2 symptoms, Mild, Moderate, or Severe) based on DSM-5 criteria at baseline, weeks 12 and 24 visits; Proportion of subjects with a 1-level categorical shift from baseline in modified DRL, at week 24; Proportion of subjects with a 2-level categorical shift from baseline in modified DRL, at week 24; Change from baseline in total alcohol consumption (TAC), defined as the mean daily alcohol consumption expressed in g/day, weeks 16-24; Change from baseline in the PHQ-9 calculated as the difference from baseline to week 24; the number of subjects with no risk alcohol consumption will be calculated, weeks 16-24; Proportion of subjects with ≥10%, ≥20%, ≥30%, ≥40%, ≥50%, ≥60%, ≥70%, ≥80%, and ≥90% reduction from baseline in monthly TAC, weeks 20-24; Change from baseline in the percent of non-drinking days (PNDD), weeks 16-24; and Change from baseline in the monthly drinks per drinking day (DDD), weeks 16-24.

### Baseline Characteristics

The baseline drinking levels for heavy drinking individuals and very heavy drinking individuals averaged 70.8 g ethanol/day and 118.5 g ethanol/day, respectively (Table 1). The heavy drinking individuals' alcohol consumption was like that of recruits reported for previous large-scale multicenter registration trials. Participants at “very high risk” for the consequences of AUD were 32.5% and 51.5% for heavy drinking individuals vs. very heavy drinking individuals, respectively, and 99% of subjects in each category consumed ≥6 heavy drinking days (HDD)/month.

TABLE 1
Demographic and Baseline Characteristics of the Study Subjects

| Heavy (n = 191) | | | Very heavy (n = 112) | | |
|---|---|---|---|---|---|
| AD04 | Placebo | p- | AD04 | Placebo | |
| (n = 99) | (n = 92) | value | (n = 57) | (n = 55) | p-value |

Demographic Characteristics

| Age | 48 | (23-78) | 50 | (23-76) | 0.31 | 50 | (27-67) | 47 | (23-73) | 0.21 |
|---|---|---|---|---|---|---|---|---|---|---|
| Gender, No. (%) | | | | | 0.88 | | | | | 0.42 |
| Men | 63 | (64%) | 57 | (62%) | | 47 | (82%) | 49 | (89%) | |
| Women | 36 | (36%) | 35 | (38%) | | 10 | (18%) | 6 | (11%) | |
| Height, cm | 173.5 | | 172.3 | | 0.32 | 175.5 | | 175.8 | | 0.86 |
| | (147.0-196.0) | | (157.0-190.0) | | | (156-190) | | (156-192) | | |
| Weight, kg | 81.6 | | 79.1 | | 0.30 | 84.9 | | 86.6 | | 0.64 |
| | (42.0-138.0) | | (48.6-127.3) | | | (55.0-121.5) | | (50.0-170.0) | | |
| BMI, kg/m² | 27.0 | | 25.8 | | 0.58 | 27.5 | | 27.9 | | 0.70 |
| | (15.1-46.1) | | (17.6-44.8) | | | (18.8-38.6) | | (17.7-51.3) | | |
| Genotype*, No. (%) | | | | | | | | | | |
| LL/TT | 39 | (39%) | 41 | (45%) | 0.56 | 28 | (49%) | 25 | (45%) | 0.71 |
| AG | 43 | (43%) | 37 | (41%) | 0.66 | 22 | (39%) | 26 | (47%) | 0.45 |
| GG | 17 | (17%) | 20 | (22%) | 0.47 | 10 | (18%) | 6 | (11%) | 0.42 |
| AC | 30 | (30%) | 31 | (34%) | 0.64 | 21 | (37%) | 15 | (27%) | 0.32 |

Measures of Alcohol Drinking

| Baseline Diagnosis | | | | | | 0.38 | | | | | >0.99 |
|---|---|---|---|---|---|---|---|---|---|---|---|
| of AUD per DSM-5 | | | | | | | | | | | |
| Moderate | | 44 | (44%) | 35 | (39%) | | 12 | (21%) | 12 | (22%) | |
| Severe | | 55 | (56%) | 55 | (61%) | | 45 | (79%) | 43 | (78%) | |
| Percent of heavy | | 65.6% | | 62.5% | | 0.95 | 76.3% | | 75.8% | | 0.93 |
| drinking days | | (14.3%-100%) | | (21.4%-100%) | | | (7.1%-100%) | | (25.0%-100%) | | |
| Total alcohol | | 70.8 | | 70.5 | | 0.95 | 118.2 | | 118.8 | | 0.96 |
| consumed, g/day | | (20.6-162.9) | | (27.0-291.0) | | | (16.7-387.0) | | (37.0-322.5) | | |
| Mean drinks per | | 8.9 | | 9.2 | | 0.67 | 14.8 | | 14.1 | | 0.59 |
| drinking day | | (3.8-22.1) | | (3.4-29.1) | | | (4.7-67.7) | | (4.7-32.3) | | |
| Risk drinking level | | | | | | 0.55 | | | | | 0.68 |
| at baseline | | | | | | | | | | | |
| Abstinence | | 0 | | 0 | | | 0 | | 0 | | |
| Very low risk | | 0 | | 0 | | | 1/57 | (2%) | 0 | | |
| Low risk | | 2/99 | (2%) | 1/92 | (1%) | | 1/57 | (1%) | 0 | | |
| Medium risk | | 32/99 | (32%) | 29/92 | (32%) | | 5/57 | (9%) | 8/55 | (15%) | |
| High risk | | 34/99 | (34%) | 40/92 | (43%) | | 17/57 | (30%) | 17/55 | (31%) | |
| Very high risk | | 31/99 | (31%) | 22/92 | (24%) | | 33/57 | (58%) | 30/55 | (55%) | |
| Heavy alcohol | | | | | | >0.99 | | | | | >0.99 |
| consumption | | | | | | | | | | | |
| patterns | | | | | | | | | | | |
| 0 | HDD | 0 | | 0 | | | 0 | | 0 | | |
| 1 | HDD | 0 | | 0 | | | 0 | | 0 | | |
| 2 | HDD | 0 | | 0 | | | 1/57 | (2%) | 0 | | |
| 3 | HDD | 0 | | 0 | | | 0 | | 0 | | |
| 4 | HDD | 1/99 | (1%) | 0 | | | 0 | | 0 | | |
| 5 | HDD | 0 | | 0 | | | 0 | | 0 | | |
| ≥6 | HDD | 98/99 | (99%) | 92/92 | (100%) | | 56/57 | (98%) | 55/55 | (100%) | |

### Results

Three hundred and three subjects (ondansetron (AD04): 156; placebo: 147) received at least one dose of the study drug and had at least one valid post-baseline assessment of the TLFB. A power calculation prior to study initiation estimated the power would be 0.95 to detect significant differences in the percent heavy drinking days (PHDD) at Months 5 and 6 between the treatment groups. Post-study power analyses on the PHDD measure indicated that the overall power was 0.533 (Field bootstrapping) (Field C A, et al. J R Stat Soc B 2007; 69(3): 369-90).

For the total cohort, the MMRM model yielded no significant main effect (p=0.55) or interaction effect of treatment x visit (p=0.23). For Months 5 and 6, the least-squares (LS) mean difference in the change in PHDD from baseline was 2.1% between the AD04 and the placebo groups (p=0.53) in favor of the AD04 group. For Month 6 only, the LS mean difference in the change in PHDD from baseline was 2.9% between the AD04 and the placebo groups (p=0.41) in favor of the AD04 group. Examination of the treatment effect in the pre-specified endophenotype of the drinking characteristic of the heavy drinking individuals showed no main effect (p=0.11) or interaction effect of treatment x visit (p=0.28) in PHDD change during Months 5 and 6 from baseline. For very heavy drinking individuals, the corresponding p-values were 0.38 and 0.59, respectively.

Planned analyses PHDD showed the average PHDD for Months 5 and 6 and each study month (Months 1-6). For Months 5 and 6 in the heavy drinking individuals' group, the difference in the LS mean change in PHDD from baseline was greater, but non-significant (LS mean difference of −7.0%; p=0.07), for the AD04 group (LS mean (SD): −45.0% (2.7%), 95% confidence interval (CI): −50.3% to −39.7%) compared with the placebo group (LS mean (SD): −38.0% (2.8%), 95% CI: −43.6% to −32.5%). The planned contrasts that examined PHDD for each month showed a significant reduction in PHDD for the AD04-treated heavy drinking individuals group compared with placebo controls during Month 1 and Month 6 of the study. During Month 1, the LS mean (SD) change in PHDD was significantly greater in the AD04 group compared with placebo (−32.0% (2.8%), 95% CI: −37.5% to −26.5% vs. −23.2% (2.9%), 95% CI: −28.9 to −17.5%; LS mean difference=−8.8%; p=0.026). Similarly, during Month 6, the LS mean (SD) change in PHDD from baseline was greater in the AD04 group compared with placebo (−46.7% (2.7%), 95% CI: −52.1% to −41.2% vs. −38.1% (2.9%), 95% CI: −43.8% to −32.5%, respectively; LS mean difference=−8.5%; p=0.03). For the very heavy drinking individuals' group, no significant effects were observed. See additional information at assets-eu.researchsquare.com/files/rs-2389870/v1/9e986b74ad9371dcd137aa5b.docx.

In response to the significant AD04-induced reduction in PHDD during Months 1 and 6, an additional analysis (not listed in the SAP) was performed to examine AD04 treatment efficacy throughout the entire treatment period. On PHDD, a Mixed Factor Repeated Measures Analysis of Variance (MFRM ANOVA) was done. Significant effects were found for Study Months alone (WL=0.6251; F[5216]=25.91; p<0.001) and the Study Months*DDD Stratum (WL=0.9246; F[5216]=3.52; p=0.004). For the heavy drinking individuals' group, the Study Month*Drug Treatment interaction effect was not found to be significant (WL=0.9576, F[5133]=1.18, p=0.323); however, the between-subjects effect of Drug Treatment was significant (F[1137]=4.57, p=0.034). This indicated that for the 6-month study period, individuals in the heavy drinking individuals' group who received AD04 had consistently reduced PHDD compared with placebo.

Given that AD04 reduced PHDD in the heavy drinking individuals' group during Months 1 and 6, a correlational analysis on PHDD values between Month 1 and Month 6 was performed. In the heavy drinking individuals' group, those who received AD04 had a significant negative correlation (r=−0.33; p=0.005) on PHDD value during Month 1 and Month 6. Additionally, a correlational analysis on the change in PHDD from baseline between Months 1 and 6 was done. The change in PHDD during Month 1 was correlated with the value during Month 6 of AD04 treatment (r=0.76; p<0.001). Additional sensitivity models examined the effects of treatment on the PHDD outcome using different statistical analytic plans and a lower DDD cut-off for women.

The effect of AD04 and placebo in the heavy drinking individuals' group on the mean (standard error (SE)) total alcohol consumed (TAC) during the 6-month clinical trial was analyzed. For the AD04 group compared with the placebo group, there was no significant effect on TAC in the very heavy drinking individuals' group (p≥0.05) for any time duration; however, there was a significant, predictable directional effect (p=0.05; one-tailed) to reduce TAC at Month 6, but not for any other duration.

The effects of AD04 vs. placebo on mean (SE) DDD during the clinical trial in the heavy drinking individuals' group were also analyzed. An MFRM ANOVA was done for the DDD variable, comprising a within-subject factor of “Time” and between-subject factors of “Treatment” and “drinking characteristic endophenotype”. The overall analysis showed a significant three-way interaction (F_{6,212}=13.64; p<0.0001). The significant three-way interaction term was decomposed by examination of the effects of “Time” and “Treatment” in heavy drinking individuals and very heavy drinking individuals. In the heavy drinking individuals' group, there was a significant effect of “Time” (F_{6,129}=30.46; p<0.001).

Individual ANOVAs were performed at each time point on data derived from the heavy drinking individuals' group. The pre-specified endpoint (i.e., the average value for Months 5 and 6) showed a non-significant effect for AD04 F_{1,131}=1.67 and p=0.097. Examination of the effects of AD04 vs. placebo treatment in the heavy drinking individuals' group during Month 6 showed a non-significant finding (F_{1,131}=1.83 and p=0.07. In the very heavy drinking individuals' group, there was no significant effect on drinks per drinking day (DDD) in the AD04 group compared with the placebo for the average value at Months 5 and 6 (p=0.072) or Month 6 (p=0.14).

The U.S. Food & Drug Administration (FDA) has indicated that a valid clinical endpoint in RDBPCTs investigating treatments for AUD is the establishment of alcohol abstinence (i.e., no drinking days (NDD)) (see e.g., Falk D E, et al. JAMA Psychiatry 2019; 76(4):374-81). In the present study, abstinence was not a stated goal. NDD, a qualitative variable, was analyzed using the non-parametric Wald's Chi-Square. The overall results showed no significant effect of AD04 compared with placebo on NDD. For the heavy drinking individuals' group, there was also no significant effect of AD04 compared with placebo for NDD for the average of Months 5 and 6 (p=0.21) and Month 6 (p=0.11).

There was a significant overall improvement in AUD severity from baseline to study end for both heavy drinking individuals and very heavy drinking individuals (p=0.04). At Month 6, for the heavy drinking individuals, the AUD severity pattern in the AD04 group was significantly different from the placebo group: (mild symptoms: AD04 group 33% vs. placebo group 39%; severe symptoms: AD04 group 10% vs. placebo group 24%) (p=0.05, Fisher's exact test). At month 6, for the very heavy drinking individuals group, the AD04 group compared with placebo showed a non-significant pattern (mild symptoms: AD04 group 33% vs. placebo group 39%; severe symptoms: AD04 group 21% vs. placebo group 26%) (p=0.10, Fishers' exact test).

For the change in WHO Quality of Life BREF, the overall treatment effect in the combined heavy drinking individuals and very heavy drinking individuals was nonsignificant for at least 1-level change (odds ratio (OR)=1.55, p=0.31), and at least 2-level change (OR=1.01, p=0.96), or an at least 3-level change (OR=1.08, p=0.79). However, in the heavy drinking individuals group, the treatment effect was significant for an at least 1-level change, with an increase in the OR of an at least 1-level downwards shift of 3.4 (95% CI: 1.03-11.45, p=0.044). The treatment effect was, however, not significant for an at least 2-level shift (OR=1.60, p=0.20) or an at least 3-level shift (OR=1.74, p=0.12). For the very heavy drinking individuals group, the treatment effect was not significant for at least a 1-, 2-, or 3-level shift.

In summary, there was a significant reduction in the monthly percentage of heavy drinking days, PHDD (−46.7% (2.7%), 95% CI: −52.1% to −41.2% vs. −38.1% (2.9%), 95% CI: −43.8% to −32.5%, respectively; LS mean difference=−8.5%; p=0.03) among AD04-treated vs. placebo-receiving heavy drinking individuals at month 6. Heavy drinking individuals were also less likely to be diagnosed with AUD [Month 1: −32.0% (2.8%), 95% CI: −37.5% to −26.5% vs. −23.2% (2.9%), 95% CI: −28.9 to −17.5%; LS mean difference=−8.8%; p=0.026)], and improved on the WHO quality of life BREF scale with a significant effect for at least a 1-level downward shift (OR=3.4; 95% CI: 1.03-11.45, p=0.044). AD04 had an exceptional safety and tolerability profile, like the placebo's. Certain information relating to this example can be found in www. researchsquare.com/article/rs-2389870/v1.pdf?c=1678907490000, or //doi.org/10.21203/rs.3.rs-2389870/v1.

### DISCUSSION

A precision medicine approach, using, i.e., administering to a subject, an effective amount of ondansetron by segregating AUD populations by the bio-genetic endophenotype of specific serotonergic genotypes and the biopsychosocial endophenotype of the severity of drinking, or both, is useful. Sellers and colleagues (Alcohol Clin Exp Res 1994; 18(4):879-85) showed in a small RDBPCT (n=71) that in the endophenotype of heavy drinking individuals (i.e., those that drank <10 standard drinks per drinking day (DDD) at enrollment), but not in the endophenotype of very heavy drinking individuals (≥10 DDD at enrollment), that low-dose ondansetron (0.5 mg/day) group compared with placebo had a trend towards less alcohol consumption. In contrast, in another small RDBPCT (n=51), high-dose ondansetron (16 mg/day) compared with placebo was inefficacious at decreasing alcohol consumption but instead increased it (Correa Filho J M et al., Addict Behav 2013; 38(4): 2044-51.). Ondansetron's therapeutic effects were most manifest at lower doses and in the bio-psychosocial endophenotype of heavy drinking individuals (those who consumed <10 DDD).

AUD is a heterogeneous disorder characterized by specific endophenotypes and diagnoses with the identification of non-overlapping psychosocial and behavioral symptoms (Johnson B. et al. Front Psychiatry 2023; 14:1271229), with a low likelihood of developing a pharmacological agent that will, robustly, treat all AUD subjects. A precision medicine approach for specific treatment-responsive endophenotypes of individuals with AUD should, however, produce a more powerful response to treat the disorder.

In this RDBPCT, in the heavy drinking individuals' group (<10 DDD), during Months 1 and 6, the mean change in the PHDD from baseline was 8.5% greater in the AD04 vs. in the placebo group. Furthermore, the COVID-19 pandemic, during which time the present study was done, might have resulted in reduced availability of alcohol products and, consequently, lower than expected alcohol consumption for all groups, thereby magnifying the placebo response and creating a “floor” effect in the ability to detect differences between the AD04 and placebo groups. In this study, the global COVID-19 pandemic may have increased the placebo response due to movement restrictions in Europe, thereby limiting the opportunity to go out and purchase alcohol or have it delivered. In the COMBINE trial (Anton R F, N Engl J Med 2008; 359(7):715-21), factors such as pill-taking, interactions with healthcare professionals, and optimism about medication effectiveness contributed significantly to the placebo response. Placebo response varied widely across 51 naltrexone and acamprosate trials, showing a negative correlation with the treatment effect size (Toneatto T, et al. Alcohol Clin Exp Res 1992; 16(3):542-6). In this study, effects of placebo on treatment outcomes that appeared to reduce the observed size of the therapeutic effect were found; however, these placebo effects appeared to be moderated by the use of BBCET treatment instead of formal psychotherapy for psychosocial support.

This RDBPCT supports AD04 as a viable precision medicine for reducing alcohol consumption in AUD subjects with specific bio-genetic and bio-psychosocial endophenotype markers of genotype and severity of drinking characteristics. There were additional indications of the beneficial effects of ondansetron (AD04) on treatment. The heavy drinking individuals AD04 group compared with the placebo group had a significant reduction in PHDD during Months 1 and 6, decreased TAC during Month 6 (one-tailed statistical test due to the predicted direction of effect), improved quality of life measures, reduced likelihood of a DSM-5 diagnosis for AUD at Month 6, and showed promising trends to reduce DDD.

This study segregated the cohort of individuals with AUD by bio-genetic and bio-psychosocial endophenotypes. This segregation allowed identification of clinically distinct endophenotypes that may be amenable to a precision medicine approach. This is consistent with the hypothesis that AD04's potential therapeutic effect was based on a complex Gene×Environment interaction, whereby “Environment” was the type of heavy drinking. The level of alcohol consumption affects the expression levels of different serotonin genotypes and clinical response to ondansetron.

Binge drinking is an important endophenotype of AUD. This broad category of endophenotype can be subdivided into heavy drinking individuals and very heavy drinking individuals, as there appears to be a distinction in binge drinking severity based on the amount of alcohol consumed within a specific time period. While the very heavy drinker group compared with the heavy drinker group did differ by segregation criterion on DDD, their actual TAC overlapped significantly, as seen in this study. Indeed, the differentiation between these endophenotypes seems to go beyond the simple frequency of heavy drinking but may include variations in behavioral and neurophysiological characteristics. These variations may be associated with the recruitment of different or supplemental brain pathways associated with neuroplasticity and may respond differentially to various medications for AUD. Indeed, preclinical research has shown that repeated binge alcohol consumption results in distinct behavioral changes and alters pharmacological responses to potential therapeutics for the treatment of AUD compared with organisms that have consistent heavy drinking for the same duration (Litten R Z, et al. Alcohol Clin Exp Res 2013; 37(12):2128-37.).

Furthermore, there is a positive correlation between alcohol consumption and use/dependency of other drugs of abuse, leading to more alcohol-related and other drug use-related problems than just alcohol consumption. In young adults, very heavy drinking is associated with polydrug use. The present RDBPCT allowed for the use of other drugs of abuse (e.g., nicotine and marijuana). Extended excessive consumption of alcohol and nicotine results in unique alterations in neurochemistry (i.e., a 4-fold increase in basal glutamate levels in the medial prefrontal cortex), enhanced drug-seeking behaviors, and alterations in neurotrophic factors (BDNF) within the nucleus accumbens shell compared with equivalent alcohol and nicotine alone consumption. Therefore, preclinical and clinical data indicate that very heavy drinking compared with heavy drinking, combined with other drugs of abuse, may result in individuals that are more resistant to pharmacotherapeutics for AUD, or may require other distinct treatment options to observe efficacious effects.

The FDA and the European Medicines Agency (EMA) have indicated that potential pharmacological treatments for AUD should improve patients' quality of life. The relevance of this goal is supported by research showing a positive association between AUD symptoms and greater negative consequences on health and quality of life, even if the individual is not consuming alcohol excessively at that time. Hence, the burden to overall health in subjects with AUD and to society is magnified if there are a high number of AUD symptoms as well as excessive alcohol consumption. The current study indicates that ondansetron (AD04) treatment appears to reduce AUD symptoms and improve the quality of life in heavy drinking individuals, which is relevant to the targeted goals of the FDA and EMA.

In this study, multiple serotonin-related SNPs resulted in AD04, compared with placebo, being efficacious at reducing multiple measures of alcohol consumption. Individuals with AUD might be highly polygenic, and the etiological pathways and genetic variations involved may vary among different populations (Sharpe K. Lancet Psychiatry 2015; 2(10):871-3.). Genome-wide association studies (GWAS) on AUD confirm its polygenic nature and the emergence of polygenic risk scores (PRS) for AUD risk assessment (Lai D, et al. Transl Psychiatry 2022; 12(1):266). GWAS and PRS are tools for predicting disease risk and identifying potential candidates for clinical applications.

This study also showed an exceptional safety and tolerability profile of ondansetron (AD04) compared with placebo, with a low incidence of adverse events (AEs), high medication compliance, and a low dropout rate. In addition, low-dose AD04 showed no significant changes in key liver biochemical parameters in individuals with AUD, suggesting it may be a safe treatment candidate for both AUD and Alcohol-associated Liver Disease (ALD) (Addolorato G, et al. Eur J Intern Med 2024; S0953-6205(24):00123-7).

There is currently no published study in alcohol literature whereby an efficacious medicine had an AE profile like placebo. For example, naltrexone is associated with high intolerability (≤15% of patients); acamprosate has been associated with high rates of study withdrawal and non-compliance due to severe gastrointestinal discomfort, with ≤30% of patients tolerating it long-term; and disulfiram can lead to potentially fatal AEs with concomitant alcohol consumption.

Ondansetron (AD04) is, therefore, a unique medicine that could significantly gain favor among non-specialist clinical practitioners, as both 6-month pill-taking and study completion exceeded 95% and 70%, respectively. A potential/likely reason for the high compliance to AD04 treatment in heavy drinking AUD subjects is that there was a significant effect as early as Month 1. The efficacy at Month 1 was associated with the efficacy of AD04 during Month 6, and for the 6-month study duration, AD04 reduced alcohol consumption. Furthermore, the psychosocial intervention platform used in this study, BBCET, has been associated with high medication and study compliance rates in several studies.

A significant barrier to the widespread treatment of patients with AUD is stigma. Stigma has resulted in only about 10% of individuals with an AUD seeking treatment, and perhaps only about 1% receiving medication (Guiraud J, et al. Eur Neuropsychopharmacol 2021; 52:18-30). Furthermore, there is great reluctance in the youth to admit the need for help with an AUD, let alone to agree to become abstinent for life. Thus, a treatment that focuses on harm reduction in currently heavily drinking individuals, perhaps especially the youth, may gain wide acceptance. Also, a medicine that is based on the genetic profile of the patient may be more socially accepted as conceptualizing AUD as a “medical problem.” Coupling an efficacious medicine with a standardized, brief psychosocial intervention that takes about the same time as an average medical visit to deliver, could change favorably how AUD disease is perceived and could increase the demand for treatment to many who would not have otherwise considered it.

### Example 2A. Effect of Different Genotypes on the Efficacy of Treating Heavy Drinkers Having Alcohol Use Disorder with Ondansetron (AD04)

The inventors herein further performed post hoc analyses on genotypes for certain clinical endpoints of results obtained from the study in Example 1. In particular, the genotypes analyzed are categorized as follows:
AC only, i.e., no other genotypes observed
AG only, i.e., no other genotypes observed
GG only, i.e., no other genotypes observed
AC and AG only, i.e., no other genotypes observed
LL and TT only, i.e., no other genotypes observed
AG+, i.e., AG positive, and may or may not have any other genotypes
AC+, i.e., AC positive, and may or may not have any other genotypes
GG+, i.e., GG positive, and may or may not have any other genotypes
AC/AG+, i.e., AC and AG positive, and may or may not have any other genotypes
LL/TT+, i.e., LL and TT positive, and may or may not have any other genotypes

The results of the analysis based on the different genotype categorizations are shown in Table 2 below. The measurement methods and statistic analysis are as shown in Example 1 herein, with differences in least square mean values between treatment group and placebo group shown in Table 2.

TABLE 2
Effects of AD04 on measures of alcohol consumption in the heavy drinking individuals'
group (Percent Heavy Drinking Days—PHDD; No Drinking Days—NDD; Change
in Total Alcohol Consumed—ΔTAC; Change in Drinks per Drinking Day—ΔDDD)

| | | PHDD | PHDD | p | p | NDD | NDD | p | p |
|---|---|---|---|---|---|---|---|---|---|
| Genotype | N | M 5&M 6 | M 6 | M 5&M 6 | M 6 | M 5&M 6 | M 6 | M 5&M 6 | M 6 |
| AC | 7 | 7.11 | 2.86 | 0.75 | 0.91 | NA | NA | NA | NA |
| AG | 17 | 4.29 | 0.03 | 0.77 | 0.99 | NA | NA | NA | NA |
| GG | 22 | −5.88 | −10.91 | 0.64 | 0.42 | 2.222 | 3.814 | 0.54 | 0.287 |
| AC/AG | 26 | −30.01 | −31.22 | 0.0045 | 0.0062 | 5.705 | 2.495 | 0.055 | 0.26 |
| LL/TT | 58 | −4.4 | −5.9 | 0.18 | 0.14 | 0.591 | 0.603 | 0.389 | 0.377 |
| AC+ | 61 | −12.51 | −14.37 | 0.03 | 0.018 | 1.623 | 2.133 | 0.365 | 0.157 |
| AG+ | 80 | −12.07 | −13.46 | 0.02 | 0.01 | 3.158 | 3.439 | 0.022 | 0.015 |
| GG+ | 37 | −11.27 | −11.92 | 0.25 | 0.25 | 4.018 | 3.094 | 0.139 | 0.16 |
| AC/AG+ | 48 | −19.96 | −20.95 | 0.6 | 0.55 | 3.042 | 3.314 | 0.071 | 0.051 |
| LL/TT+ | 77 | −3.32 | −3.99 | 0.003 | 0.004 | 0.744 | 0.762 | 0.55 | 0.582 |
| All | 191 | −7 | −8.8 | 0.07 | 0.031 | 1.495 | 1.629 | 0.21 | 0.11 |

| | ΔTAC | ΔTAC | p | p | ΔDDD | ΔDDD | p | p |
|---|---|---|---|---|---|---|---|---|
| Genotype | M 5&M 6 | M 6 | M 5&M 6 | M 6 | M 5&M 6 | M 6 | M 5&M 6 | M 6 |
| AC | 25.39 | 18 | 0.19 | 0.38 | 3.2678 | 1.6063 | 0.75 | 0.88 |
| AG | 8.5 | 7.89 | 0.47 | 0.5 | −3.9799 | −3.3817 | 0.03 | 0.067 |
| GG | 1.9 | −0.98 | 0.9 | 0.95 | −2.1263 | −1.9583 | 0.159 | 0.195 |
| AC/AG | −23.28 | −24.45 | 0.038 | 0.048 | −3.2925 | −2.7577 | 0.038 | 0.116 |
| LL/TT | 5.1 | 5.3 | 0.46 | 0.49 | 0.2436 | 0.1458 | 0.75 | 0.86 |
| AC+ | −8.25 | −10.67 | 0.15 | 0.07 | −0.7715 | −1.1061 | 0.44 | 0.27 |
| AG+ | −6.27 | −7.6 | 0.24 | 0.16 | −2.0947 | −1.9635 | 0.008 | 0.016 |
| GG+ | −16.67 | −17.64 | 0.052 | 0.039 | −1.8572 | −1.6402 | 0.11 | 0.17 |
| AC/AG+ | −15.1 | −16.38 | 0.0306 | 0.0261 | −2.0624 | −1.9053 | 0.048 | 0.076 |
| LL/TT+ | 1.25 | 1.44 | 0.83 | 0.81 | 0.7018 | 0.5346 | 0.371 | 0.504 |
| All | −3.07 | −4.3 | 0.39 | 0.23 | −0.8378 | −0.9335 | 0.1 | 0.071 |

M: Month; p: p-value.

For PHDD, in the heavy drinking individuals group, data analysis showed a significant difference between the AD04 group and the placebo group among subjects with an AC/AG, AC+, AG+, or LL/TT+ for the average PHDD at Months 5 and 6 and for Month 6. There was no significant effect of AD04 vs. placebo treatment among the heavy drinking individuals with only a single nucleotide polymorphism (SNP) (AC alone, AG alone, or GG alone). There was a similar genotype association with the TAC analysis. In the heavy drinking individuals group, AD04 compared with placebo reduced TAC significantly among individuals with the AC/AG, AC/AG+, and GG+ profile (Month 6 only).

For the Alcohol Consumption Risk Responder analysis, AD04 compared with placebo increased the rate of NDD for the average of Months 5 and 6 in AC/AG, GG+, and LL/TT (Table 2). In the heavy drinking individuals group, there was a significant increase in the AD04 group compared with the placebo group on NDD for all subjects except those with only the AC genotype (point estimate −2.025; p=0.05). For DDD, in the heavy drinking individuals group, there was a significant reduction for the AD04 group compared with the placebo group on the average of Months 5 and 6 and at Month 6 in subjects with the AG or AC/AG genotypes (Table 2).

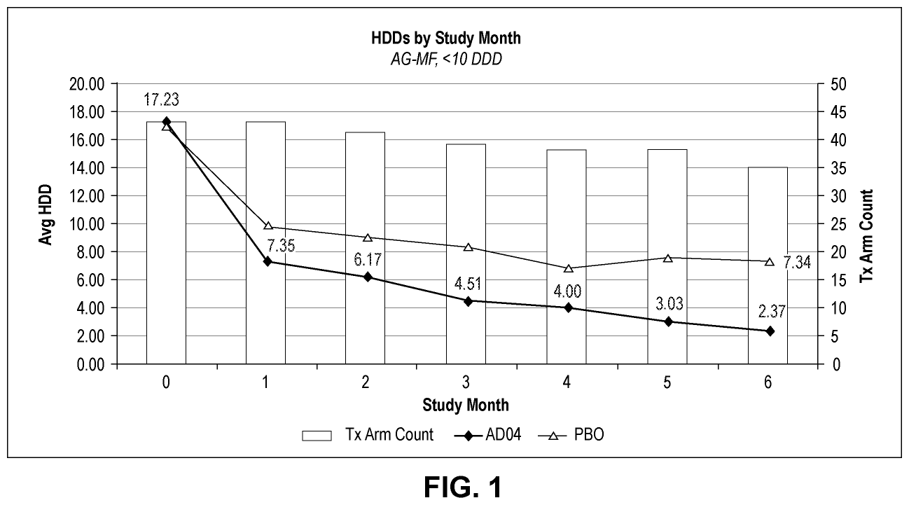

FIGS. 1 through 4 show the average number of heavy drinking days (HDD) by study month for heavy drinkers (<10 drinks per drinking day (DDD)) of a genotype for individuals receiving ondansetron (AD04) and individuals receiving placebo (PBO).

FIG. 1 shows results for individuals having the AG genotype. At Month 0 (the start of the Phase 3 clinical trial), the average number of HDDs were similar for the AD04 and PBO groups, as expected. The average numbers of HDDs decreased for both the AD04 and PBO groups for Months 1 through 4, however, the numbers diverged, with a greater reduction in HDDs for those individuals receiving ondansetron (AD04) than for those receiving placebo (PBO). Moreover, for Months 5 and 6, the HDDs continued to decrease for those individuals receiving ondansetron (AD04), whereas the HDDs plateaued for those receiving placebo (PBO).

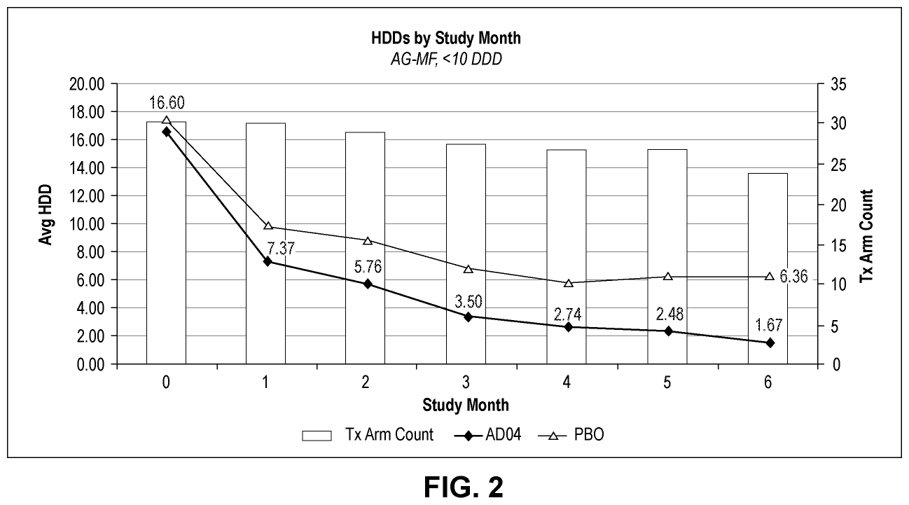

FIG. 2 shows results for individuals having the AC genotype. At Month 0 (the start of the Phase 3 clinical trial), the average number of HDDs were similar for the AD04 and PBO groups, as expected. The average numbers of HDDs decreased for both the AD04 and PBO groups for Months 1 through 4, however, the numbers diverged, with a greater reduction in HDDs for those individuals receiving ondansetron (AD04) than for those receiving placebo (PBO). Moreover, for Months 5 and 6, the HDDs continued to decrease for those individuals receiving ondansetron (AD04), whereas the HDDs plateaued for those receiving placebo (PBO).

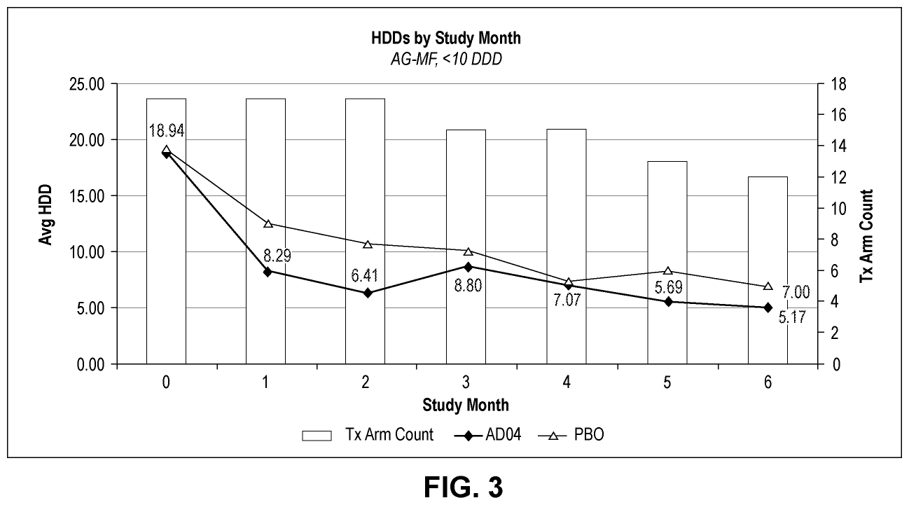

FIG. 3 shows results for individuals having the GG genotype. At Month 0 (the start of the Phase 3 clinical trial), the average number of HDDs were similar for the AD04 and PBO groups, as expected. The average numbers of HDDs decreased for both the AD04 and PBO groups from Month 0 to Months 5 and 6. For Months 5 and 6, the HDDs continued to decrease for those individuals receiving ondansetron (AD04), whereas the HDDs plateaued for those receiving placebo (PBO).

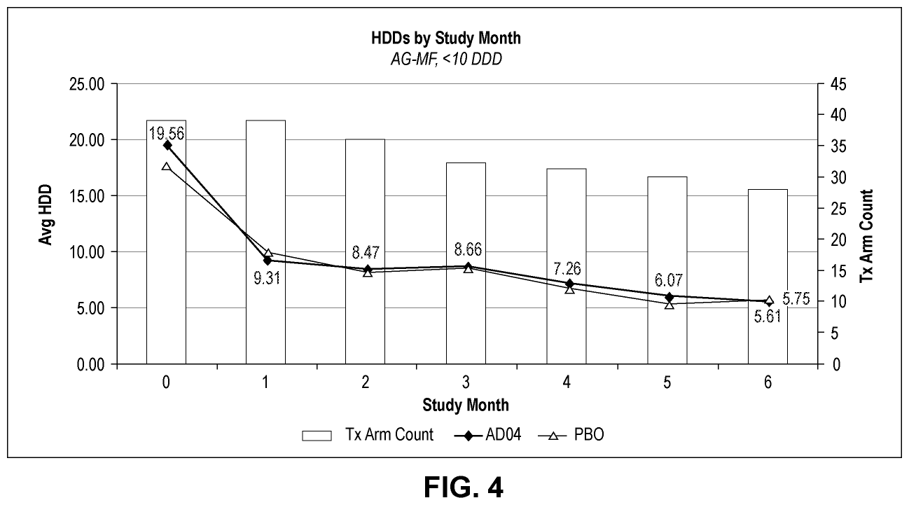

FIG. 4 shows results for individuals having the LL/TT genotype. At Month 0 (the start of the Phase 3 clinical trial), the average number of HDDs were similar for the AD04 and PBO groups, as expected. The average numbers of HDDs decreased for both the AD04 and PBO groups from Month 0 to Month 2, stayed similar from Month 2 to Month 3, decreased from Month 3 to Month 5, and stayed similar from Month 5 to Month 6. However, the average number of HDDs in a given month were similar for the group of individuals receiving ondansetron (AD04) and for the group of individuals receiving placebo (PBO).

Ondansetron's (AD04's) therapeutic effects on PHDD and quality of life were still improving at Month 6, suggesting that a longer course of treatment, such as 9 to 12 months, could have even greater beneficial effects.

In conclusion, the results provide support for ondansetron (AD04) for the treatment of currently alcohol (ethanol) heavy drinking individuals who possess a genetic profile targeted to selected genotypes in the serotonin transporter and serotonin-3AB receptor complex, such as the AG+ or the GG+ patients.

### Example 2B. Analysis of Effect of Different Genotypes on the Efficacy of Treating Heavy Drinkers Having Alcohol Use Disorder with Ondansetron (AD04)

The inventors herein performed a further post hoc analyses on genotypes for certain clinical endpoints of results obtained from the study in Example 1. In this analysis, the US definition of 14 g for standard drink is used. The end point is the response to treatment defined no (zero) heavy drinking days during the months 5 and 6 from the treatment initiation. The population-level summary is the proportion of responders in each arm. Patients with missing months 5 and 6 data were excluded from the subgroup analysis. The log-odds ratio was used for subgroup analysis, logs-odds ratio of 0 means no effect and ≥0 means treatment is better.

The initial observation of this analysis shows, consistent with the previous analysis, that heavy drinkers (baseline drinking level of less than 10 drinks per drinking day) with lower drinking level are associated with better treatment response, and heavy drinkers with baseline drinking level of less than 8 drinks per drinking day has an even higher log odds-ratio.

The genotype subgroups are further analyzed with the following 15 different groups:

| Biomarker positive population for adaptive | | |
|---|---|---|
| enrichment | Definition | N (%) |
| AG predict higher response | | |
| 1. AG+ and not LL/TT whatever others | AG = Y, GG = N, LL/TT = N | 89 (39.7%) |
| 2. AG+ and not LL and not TT whatever others | AG = Y, GG = N, LL = N, TT = N | 58 (25.9%) |
| 3. AG+ whatever others | AG = Y, GG = N | 99 (44.2%) |
| A-GG the response | | |
| 4. GG+ and not LL/TT and not AC+ | GG = Y, AC = N, LL/TT = N | 27 (12.1%) |
| 5. GG+ and not LL and not TT not AC+ | GG = Y, AC = N, LL = N, TT = N | 20 (8.9%) |
| B and A-GG- predict higher response | | |
| 6. AC+ and not LL/TT whatever others | AC = Y, GG = N, LL/TT = N | 69 (30.8%) |
| 7. AC+ and not LL and not TT whatever others | AC = Y, GG = N, LL = N, TT = N | 45 (20.1%) |
| A-AG and B predict higher response | | |
| 8. AG+ and AC+ and not LL/TT and not GG+ | AG =Y, AC = Y, GG = N, LL/TT = N | 60 (26.8%) |
| 9. AG+ and AC+ and not LL and not TT | AG = Y, AC = Y, GG = N, LL = N, | 40 (17.9%) |
| and not GG+ | TT = N | |
| 10. AG+ and AC+ and not GG+ | AG = Y, AC = Y, GG = N | 63 (28.1%) |
| LL and TT predict lower response | | |
| 11. Both LL+ and TT+ whatever others | LL = Y, TT = Y | 96 (42.9%) |
| 12. Either LL+ or TT+ or both + whatever others | LL = Y or TT = Y | 138 (61.6%) |
| A-AG and/or B predict higher response | | |
| 13. AG+ and/or AC+ (at least one of them) | (AG = Y or AC = Y), GG = N, | 98 (43.8%) |
| and not LL/TT and not GG+ | LL/TT = N | |
| 14. AG+ and/or AC+ (at least one of them) | (AG = Y or AC = Y), GG = N, | 63 (28.1%) |
| and not LL and not TT and not GG+ | LL = N, TT = N | |
| 15. AG+ and/or AC+ (at least one of them) | (AG = Y or AC = Y), GG = N | 109 (48.7%) |
| and not GG+ | | |

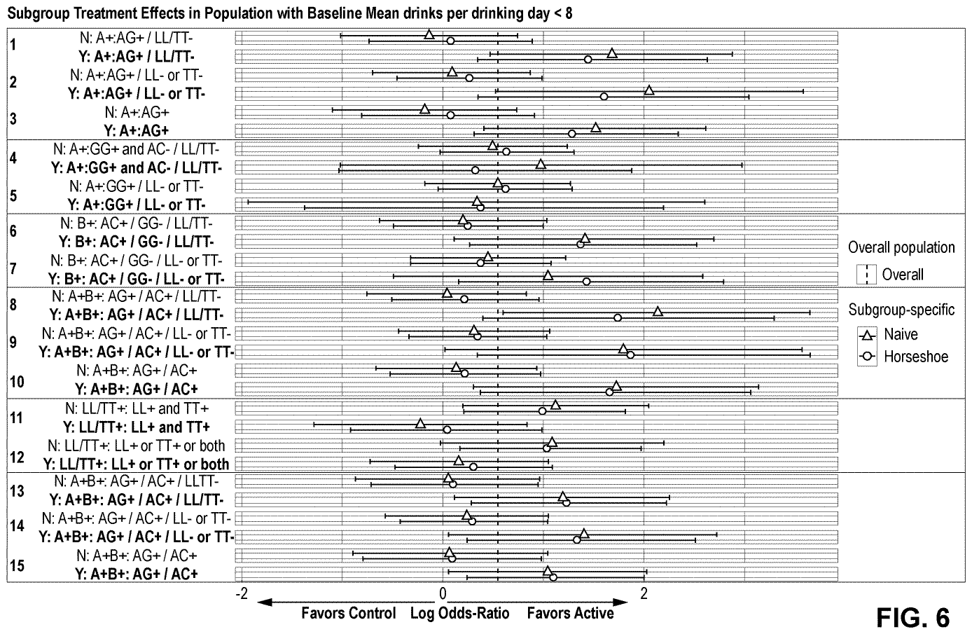

The genetic subgroup treatment effects were analyzed for population with baseline mean drinks per drinking day less than 8. The results are shown in FIG. 6 with both naïve and regularized horseshoe method, see, e.g., Carvalho, C., Polson, N., and Scott, J. (2009). “Handling sparsity via the horseshoe.” Journal of Machine Learning Research W&CP, 5(73-80).

This analysis shows that AG and AC are associated with better response to treatment, whereas GG, LL and TT are associated with lower response rates. Based on this analysis, responders can include Biomarker+ patients with AG+ or AC+(or both), and non-responders can include Biomarker+ patients with LL or TT (or both), which may be excluded in a treatment according to the methods herein. As such, biomarker-positive population can be patients with AG+; patients with AG+, AC+, or both; and/or patients without LL, TT, or both.

Lastly, it was also found that Patients with more than 25% change in drinking levels (from the time of screening to baseline) tend to show higher response.

Additional post hoc analysis also confirmed that AG or AG+AC is associated with better response to treatment whereas LL and TT are associated with lower response rates.

### Example 3. Responder Analysis of Subjects with AUD Treated with Ondansetron (AD04)

In this example, an analysis is presented of whether there are certain genotypes of subjects that can be characterized as responders to treatment with ondansetron in the phase III clinical trial of Example 1.

This analysis used the FDA specified endpoint of reduction of heavy drinking days, defined as the percentage of patients that have zero heavy drinking days (PNHDD) during the efficacy observation period of months 5 and 6, for ondansetron (AD04) vs. placebo compared to baseline at study entry.

The analysis of the PNHDD endpoint of the Phase 3 Study in Example 1 for heavy drinkers, DDD<10, at Months 5 and 6, shows that the AG+ polymorphism demonstrated a statistically significant difference at month's 5&6 compared to placebo (p=0.021). Additionally, the GG+ polymorphism in this analysis showed promising trends toward achieving the PNHDD endpoint. There was a substantial difference between the treatment and placebo arms in the percentage of patients that achieved zero heavy drinking days within the AG and GG polymorphism groups. In the same post hoc analysis comparing genotype polymorphisms against PNHDD for LL/TT no statistically significant difference was seen. The genotype definitions of AG+, GG+, and LL/TT are described in Example 2.

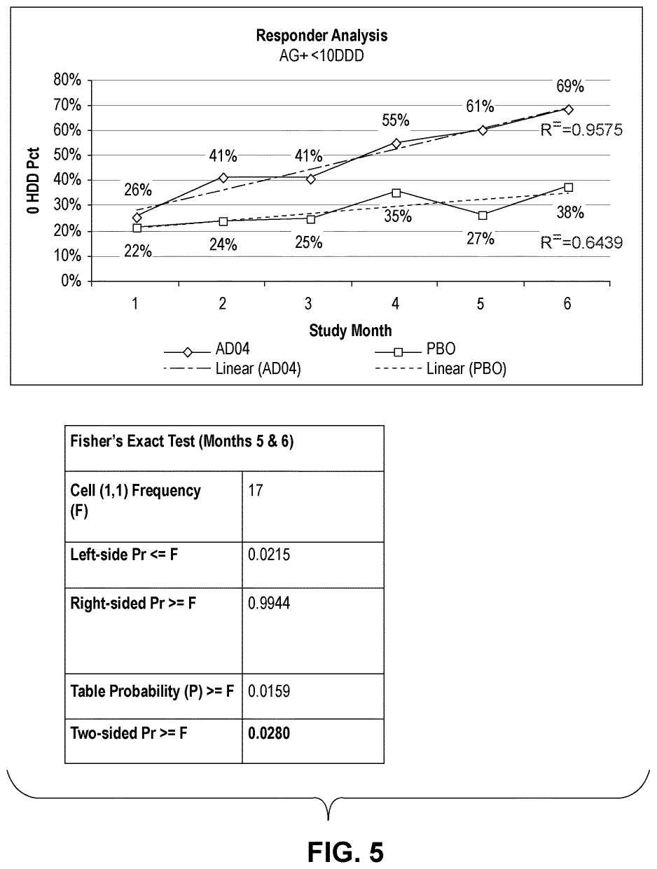

FIG. 5 shows a graph of percentage of AG+ patients achieving zero (no) HDD over the course of 6 months. Statistical data are also shown using Fisher's exact test. At Month 1, the percentages of patients achieving zero HDD are similar. The percentage of patients achieving zero HDD increased from Month 1 to Month 6, reaching 69% at Month 6, for the group administered ondansetron (AD04), much higher (greater) than the 38% observed at Month 6 for the placebo treated group (PBO).

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A method of treating a subject having alcohol use disorder to achieve two or more of the following effects (a)-(e) in one or both of the last two months of a treatment period, if the treatment period is five months or below, or in month 5 and/or month 6 if the treatment period is six months or longer:
(a) zero heavy drinking days (HDDs), wherein an HDD is a day with ethanol consumption of 60 grams or more for males and 40 grams or more for females;
(b) a reduction of percent heavy drinking days of at least 10% in addition to placebo effect, compared to that observed for the 4 weeks prior to the treatment period (“baseline”);
(c) at least two no alcohol drinking days (“NDDs” or “abstinent days”) in addition to placebo effect;
(d) a reduction of total alcohol consumption of at least 5% in addition to placebo effect, compared to baseline; and
(e) a reduction of at least one drink per drinking day in addition to placebo effect, compared to baseline, wherein each drink contains about 10 grams of ethanol;
the method comprising administering to the subject an effective amount of ondansetron, wherein:
the subject is characterized as having the following baseline characteristics:
(1) ≥6 HDDs;
(2) consuming less than 10 drinks per drinking day;
(3) having an average alcohol consumption at a medium risk level, which is ≥40 grams of ethanol per day for males and ≥20 grams of ethanol per day for females; and
(4) ≤14 consecutive abstinent days; and
(5) the subject has a genotype of AG+.

2. The method of claim 1, wherein the subject does not have a genotype of LL/TT, which is rs4795541-LL genotype of the insertion-deletion polymorphism (5′-HTTLPR) in the 5′-regulatory region and rs1042173-TT SNP in the 3′-untranslated region of SLC6A4 gene that encodes the serotonin transporter.

3. The method of claim 1, wherein the subject has a genotype of AG and at least one other SNP at the HTR3 AB complex.

4. The method of claim 1, wherein the subject has a genotype of AG and AC (rs17614942-AC in HTR3B).

5. The method of claim 1, wherein during the treatment period, the ondansetron is administered to the subject daily in an amount ranging from about 0.25 mg to about 1 mg.

6. The method of claim 1, wherein during the treatment period, the ondansetron is administered to the subject twice a day, with each dose of about 0.33 mg.

7. The method of claim 1, wherein the treatment period is at least six months.

8. The method of claim 7, wherein the ondansetron is administered to the subject such that the subject has zero HDD in month 6 of the treatment period.

9. The method of claim 7, wherein the ondansetron is administered to the subject such that the subject has a reduction of percent heavy drinking days in months 5 and/or 6 of the treatment period by at least 12% in addition to placebo effect, compared to baseline.

10. The method of claim 7, wherein the ondansetron is administered to the subject such that the subject has 2-4 days of no drinking days in addition to placebo effect in month 6 of the treatment period.

11. The method of claim 7, wherein the ondansetron is administered to the subject such that the subject has a reduction of total alcohol consumption of 7-30% in addition to placebo effect in month 6 of the treatment period compared to baseline.

12. The method of claim 7, wherein the ondansetron is administered to the subject such that the subject has a reduction of 1-3 drinks per drinking day in addition to placebo effect in month 6 of the treatment period compared to baseline.

13. A method of treating alcohol use disorder, the method comprising
administering ondansetron to a subject in need of treatment who belongs to a patient population characterized as (i) being heavy drinkers; and (ii) having a genotype of AG+;
wherein the ondansetron is administered to the subject with a dosing regimen that is effective in treating the patient population such that the percentage of the patient population having zero HDD (PNHDD) in one or both of the last two months of a treatment period, if the treatment period is five months or below, or in month 5 and/or month 6 if the treatment period is six months or longer, is higher than that of a placebo-controlled patient population,
wherein an HDD is a day with ethanol consumption of 60 grams or more for males and 40 grams or more for females,
wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days.

14. A method of increasing the likelihood of achieving zero heavy drinking days (HDDs) in one or both of the last two months of a treatment period, if the treatment period is five months or below, or in month 5 and/or month 6 if the treatment period is six months or longer, wherein an HDD is a day with ethanol consumption of 60 grams or more for males and 40 grams or more for females, the method comprising:
administering ondansetron to a subject who belongs to a patient population characterized as (i) being heavy drinkers; and (ii) having a genotype of AG+;
wherein the ondansetron is administered to the subject with a dosing regimen that is effective in treating the patient population such that the percentage of the patient population having zero HDD (PNHDD) in one or both of the last two months of the treatment period, if the treatment period is five months or below, or in month 5 and/or month 6 if the treatment period is six months or longer, is higher than that of a placebo-controlled patient population,
wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days.

15. The method of claim 13, wherein all subjects of the patient population have a genotype of AG and at least one other SNP at the HTR3 AB complex.

16. The method of claim 13, wherein all subjects of the patient population have a genotype of AG and AC (rs17614942-AC in HTR3B).

17. The method of claim 13, wherein no subject of the patient population has a genotype of LL/TT, which is rs4795541-LL genotype of the insertion-deletion polymorphism (5′-HTTLPR) in the 5′-regulatory region and rs1042173-TT SNP in the 3′-untranslated region of SLC6A4 gene that encodes the serotonin transporter.

18. The method of claim 13, wherein the treatment period is at least six months.

19. The method of claim 18, wherein the dosing regimen is effective in treating the patient population to achieve a reduction of percent heavy drinking days (PHDD) in months 5 and/or 6 of the treatment period compared to baseline, wherein the reduction of PHDD in the patient population is statistically significantly higher than that of the placebo-controlled patient population or a patient population having only a single SNP selected from AC alone, AG alone, or GG alone.

20. The method of claim 18, wherein the dosing regimen is effective in treating the patient population to achieve 2-4 more days of no drinking days (NDD) in months 5 and/or 6 of the treatment period than that of the placebo-controlled patient population.

21. The method of claim 18, wherein the dosing regimen is effective in treating the patient population to achieve a reduction of total alcohol consumption (TAC) in months 5 and/or 6 of the treatment period, wherein the reduction of TAC in the patient population is higher than that of the placebo-controlled patient population.

22. The method of claim 18, wherein the dosing regimen is effective in treating the patient population to achieve a reduction of drinks per drinking day (DDD) in months 5 and/or 6 of the treatment period, wherein the reduction of DDD in the patient population is higher than that of the placebo-controlled patient population.

23. The method of claim 18, wherein when compared to the placebo-controlled patient population, the dosing regimen is effective in treating the patient population to achieve (a) a reduction of percent heavy drinking days in months 5 and/or 6 of the treatment period compared to baseline by at least 12% greater; (b) 2-4 more days of no drinking days in month 6 of the treatment period; (c) a reduction of total alcohol consumption of in month 6 of the treatment period compared to baseline by 7-30% greater; and (d) a reduction of 1-3 more drinks per drinking day in month 6 of the treatment period compared to baseline.

24. The method of claim 13, wherein during the treatment period, the ondansetron is administered to the subject daily in an amount ranging from about 0.25 mg to about 1 mg.

25. The method of claim 13, wherein during the treatment period, the ondansetron is administered to the subject twice a day, with each dose of about 0.33 mg.

26. The method of claim 13, further comprising determining or having determined the subject as belonging to the patient population.

27. A method of predicting the likelihood of a subject having alcohol use disorder achieving zero heavy drinking days (HDDs) in one or both of the last two months of a treatment period, if the treatment period is five months or below, or in month 5 and/or month 6 if the treatment period is six months or longer, wherein an HDD is a day with ethanol consumption of 60 grams or more for males and 40 grams or more for females, the method comprising:
(1) administering ondansetron to a patient population characterized as (i) being heavy drinkers; and (ii) having a genotype of AG+;
(2) identifying the patient population as responsive to ondansetron treatment if the ondansetron can be administered with a dosing regimen that is effective in treating the patient population such that the percentage of the patient population having zero HDD (PNHDD) in one or both of the last two months of the treatment period, if the treatment period is five months or below, or in month 5 and/or month 6 if the treatment period is six months or longer, is higher than that of a placebo-controlled patient population,
wherein a heavy drinker has the following characteristics observed for the 4 weeks prior to the treatment period (“baseline”): ≥6 HDDs; consuming less than 10 drinks per drinking day; having an average alcohol consumption at a medium risk level, which is ≥40 grams of ethanol per day for males and ≥20 grams of ethanol per day for females; and having ≤14 consecutive abstinent (no alcohol drinking) days; and
3) determining or having determined whether the subject belongs to the patient population by comparing the genotype and drinking pattern with those of the patient population,
wherein a subject belonging to the patient population has a higher chance of achieving zero HDD.

28. A method of treating alcohol use disorder, the method comprising:
(1) determining or having determined a subject in need of treatment as having a higher chance of achieving zero HDD according to the method of claim 27, and
(2) administering the subject ondansetron with a dosing regimen that is effective in treating the patient population such that the percentage of the patient population having zero HDD (PNHDD) in one or both of the last two months of the treatment period, if the treatment period is five months or below, or in month 5 and/or month 6 if the treatment period is six months or longer, is higher than that of a placebo-controlled patient population.
